SEQUENCE LISTING (436 sequences)

SEQ ID NO: 1 - protein, 21 aa, Artificial sequence
  Synthetic encephalitogenic immunodominant peptide MOG34-56
IGPRHPIRALVGDEVELPCRI

SEQ ID NO: 2 - protein, 20 aa, Artificial sequence
  A myelin protein unique epitope containing peptide
RSRGAEATESKLSGGFRWTQ

SEQ ID NO: 3 - protein, 20 aa, Artificial sequence
  A myelin protein unique epitope containing peptide
SKLSGGFRWTQKTSPTQGDS

SEQ ID NO: 4 - protein, 20 aa, Artificial sequence
  A myelin protein unique epitope containing peptide
TQKTSPTQGDSAITHSLYRM

SEQ ID NO: 5 - protein, 20 aa, Artificial sequence
  A myelin protein unique epitope containing peptide
DSAITHSLYRMIFLTALPLF

SEQ ID NO: 6 - protein, 20 aa, Artificial sequence
  A myelin protein unique epitope containing peptide
RMIFLTALPLFWIMISASRG

SEQ ID NO: 7 - protein, 20 aa, Artificial sequence
  A myelin protein unique epitope containing peptide
LFWIMISASRGGHWGAWMPS

SEQ ID NO: 8 - protein, 20 aa, Artificial sequence
  A myelin protein unique epitope containing peptide
RGGHWGAWMPSSISAFEGTC

SEQ ID NO: 9 - protein, 20 aa, Artificial sequence
  A myelin protein unique epitope containing peptide
PSSISAFEGTCVSIPCRFDF

SEQ ID NO: 10 - protein, 20 aa, Artificial sequence
  A myelin protein unique epitope containing peptide
TCVSIPCRFDFPDELRPAVV

SEQ ID NO: 11 - protein, 20 aa, Artificial sequence
  A myelin protein unique epitope containing peptide
DFPDELRPAVVHGVWYFNSP

SEQ ID NO: 12 - protein, 20 aa, Artificial sequence
  A myelin protein unique epitope containing peptide
VVHGVWYFNSPYPKNYPPVV

SEQ ID NO: 13 - protein, 20 aa, Artificial sequence
  A myelin protein unique epitope containing peptide
SPYPKNYPPVVFKSRTQVVH

SEQ ID NO: 14 - protein, 20 aa, Artificial sequence
  A myelin protein unique epitope containing peptide
VVFKSRTQVVHESFQGRSRL

SEQ ID NO: 15 - protein, 20 aa, Artificial sequence
  A myelin protein unique epitope containing peptide
VHESFQGRSRLLGDLGLRNC

SEQ ID NO: 16 - protein, 20 aa, Artificial sequence
  A myelin protein unique epitope containing peptide
RLLGDLGLRNCTLLLSNVSP

SEQ ID NO: 17 - protein, 20 aa, Artificial sequence
  A myelin protein unique epitope containing peptide
NCTLLLSNVSPELGGKYYFR

SEQ ID NO: 18 - protein, 20 aa, Artificial sequence
  A myelin protein unique epitope containing peptide
SPELGGKYYFRGDLGGYNQY

SEQ ID NO: 19 - protein, 20 aa, Artificial sequence
  A myelin protein unique epitope containing peptide
FRGDLGGYNQYTFSEHSVLD

SEQ ID NO: 20 - protein, 20 aa, Artificial sequence
  A myelin protein unique epitope containing peptide
QYTFSEHSVLDIVNTPNIVV

SEQ ID NO: 21 - protein, 20 aa, Artificial sequence
  A myelin protein unique epitope containing peptide
LDIVNTPNIVVPPEVVAGTE

SEQ ID NO: 22 - protein, 20 aa, Artificial sequence
  A myelin protein unique epitope containing peptide
VVPPEVVAGTEVEVSCMVPD

SEQ ID NO: 23 - protein, 20 aa, Artificial sequence
  A myelin protein unique epitope containing peptide
TEVEVSCMVPDNCPELRPEL

SEQ ID NO: 24 - protein, 20 aa, Artificial sequence
  A myelin protein unique epitope containing peptide
PDNCPELRPELSWLGHEGLG

SEQ ID NO: 25 - protein, 20 aa, Artificial sequence
  A myelin protein unique epitope containing peptide
ELSWLGHEGLGEPAVLGRLR

SEQ ID NO: 26 - protein, 20 aa, Artificial sequence
  A myelin protein unique epitope containing peptide
LGEPAVLGRLREDEGTWVQV

SEQ ID NO: 27 - protein, 20 aa, Artificial sequence
  A myelin protein unique epitope containing peptide
LLEDEGTWVQVSLLHFVPTR

SEQ ID NO: 28 - protein, 20 aa, Artificial sequence
  A myelin protein unique epitope containing peptide
QVSLLHFVPTREANGHRLGC

SEQ ID NO: 29 - protein, 20 aa, Artificial sequence
  A myelin protein unique epitope containing peptide
TREANGHRLGCQASFPNTTL

SEQ ID NO: 30 - protein, 20 aa, Artificial sequence
  A myelin protein unique epitope containing peptide
GCQASFPNTTLQFEGYASMD

SEQ ID NO: 31 - protein, 20 aa, Artificial sequence
  A myelin protein unique epitope containing peptide
TLQFEGYASMDVKYPPVIVE

SEQ ID NO: 32 - protein, 20 aa, Artificial sequence
  A myelin protein unique epitope containing peptide
MDVKYPPVIVEMNSSVEAIE

SEQ ID NO: 33 - protein, 20 aa, Artificial sequence
  A myelin protein unique epitope containing peptide
VEMNSSVEAIEGSHVSLLCG

SEQ ID NO: 34 - protein, 20 aa, Artificial sequence
  A myelin protein unique epitope containing peptide
IEGSHVSLLCGADSNPPPLL

SEQ ID NO: 35 - protein, 20 aa, Artificial sequence
  A myelin protein unique epitope containing peptide
CGADSNPPPLLTWMRDGTVL

SEQ ID NO: 36 - protein, 20 aa, Artificial sequence
  A myelin protein unique epitope containing peptide
LLTWMRDGTVLREAVAESLL

SEQ ID NO: 37 - protein, 20 aa, Artificial sequence
  A myelin protein unique epitope containing peptide
VLREAVAESLLLELEEVTPA

SEQ ID NO: 38 - protein, 20 aa, Artificial sequence
  A myelin protein unique epitope containing peptide
LLLELEEVTPAEDGVYACLA

SEQ ID NO: 39 - protein, 20 aa, Artificial sequence
  A myelin protein unique epitope containing peptide
PAEDGVYACLAENAYGQDNR

SEQ ID NO: 40 - protein, 20 aa, Artificial sequence
  A myelin protein unique epitope containing peptide
LAENAYGQDNRTVGLSVMYA

SEQ ID NO: 41 - protein, 20 aa, Artificial sequence
  A myelin protein unique epitope containing peptide
NRTVGLSVMYAPWKPTVNGT

SEQ ID NO: 42 - protein, 20 aa, Artificial sequence
  A myelin protein unique epitope containing peptide
YAPWKPTVNGTMVAVEGETV

SEQ ID NO: 43 - protein, 20 aa, Artificial sequence
  A myelin protein unique epitope containing peptide
GTMVAVEGETVSILCSTQSN

SEQ ID NO: 44 - protein, 20 aa, Artificial sequence
  A myelin protein unique epitope containing peptide
TVSILCSTQSNPDPILTIFK

SEQ ID NO: 45 - protein, 20 aa, Artificial sequence
  A myelin protein unique epitope containing peptide
SNPDPILTIFKEKQILSTVI

SEQ ID NO: 46 - protein, 20 aa, Artificial sequence
  A myelin protein unique epitope containing peptide
FKEKQILSTVIYESELQLEL

SEQ ID NO: 47 - protein, 20 aa, Artificial sequence
  A myelin protein unique epitope containing peptide
VIYESELQLELPAVSPEDDG

SEQ ID NO: 48 - protein, 20 aa, Artificial sequence
  A myelin protein unique epitope containing peptide
ELPAVSPEDDGEYWCVAENQ

SEQ ID NO: 49 - protein, 20 aa, Artificial sequence
  A myelin protein unique epitope containing peptide
DGEYWCVAENQYGQRATAFN

SEQ ID NO: 50 - protein, 20 aa, Artificial sequence
  A myelin protein unique epitope containing peptide
NQYGQRATAFNLSVEFAPVL

SEQ ID NO: 51 - protein, 20 aa, Artificial sequence
  A myelin protein unique epitope containing peptide
FNLSVEFAPVLLLESHCAAA

SEQ ID NO: 52 - protein, 20 aa, Artificial sequence
  A myelin protein unique epitope containing peptide
VLLLESHCAAARDTVQCLCV

SEQ ID NO: 53 - protein, 20 aa, Artificial sequence
  A myelin protein unique epitope containing peptide
AARDTVQCLCVVKSNPEPSV

SEQ ID NO: 54 - protein, 20 aa, Artificial sequence
  A myelin protein unique epitope containing peptide
CVVKSNPEPSVAFELPSRNV

SEQ ID NO: 55 - protein, 20 aa, Artificial sequence
  A myelin protein unique epitope containing peptide
SVAFELPSRNVTVNESEREF

SEQ ID NO: 56 - protein, 20 aa, Artificial sequence
  A myelin protein unique epitope containing peptide
NVTVNESEREFVYSERSGLV

SEQ ID NO: 57 - protein, 20 aa, Artificial sequence
  A myelin protein unique epitope containing peptide
EFVYSERSGLVLTSILTLRG

SEQ ID NO: 58 - protein, 20 aa, Artificial sequence
  A myelin protein unique epitope containing peptide
LVLTSILTLRGQAQAPPRVI

SEQ ID NO: 59 - protein, 20 aa, Artificial sequence
  A myelin protein unique epitope containing peptide
RGQAQAPPRVICTARNLYGA

SEQ ID NO: 60 - protein, 20 aa, Artificial sequence
  A myelin protein unique epitope containing peptide
VICTARNLYGAKSLELPFQG

SEQ ID NO: 61 - protein, 20 aa, Artificial sequence
  A myelin protein unique epitope containing peptide
GAKSLELPFQGAHRLMWAKI

SEQ ID NO: 62 - protein, 20 aa, Artificial sequence
  A myelin protein unique epitope containing peptide
QGAHRLMWAKIGPVGAVVAF

SEQ ID NO: 63 - protein, 20 aa, Artificial sequence
  A myelin protein unique epitope containing peptide
KIGPVGAVVAFAILIAIVCY

SEQ ID NO: 64 - protein, 20 aa, Artificial sequence
  A myelin protein unique epitope containing peptide
AFAILIAIVCYITQTRRKKN

SEQ ID NO: 65 - protein, 20 aa, Artificial sequence
  A myelin protein unique epitope containing peptide
CYITQTRRKKNVTESPSFSA

SEQ ID NO: 66 - protein, 20 aa, Artificial sequence
  A myelin protein unique epitope containing peptide
KNVTESPSFSAGDNPPVLFS

SEQ ID NO: 67 - protein, 20 aa, Artificial sequence
  A myelin protein unique epitope containing peptide
SAGDNPPVLFSSDFRISGAP

SEQ ID NO: 68 - protein, 20 aa, Artificial sequence
  A myelin protein unique epitope containing peptide
FSSDFRISGAPEKYESERRL

SEQ ID NO: 69 - protein, 20 aa, Artificial sequence
  A myelin protein unique epitope containing peptide
APEKYESERRLGSERRLLGL

SEQ ID NO: 70 - protein, 20 aa, Artificial sequence
  A myelin protein unique epitope containing peptide
RLGSERRLLGLRGEPPELDL

SEQ ID NO: 71 - protein, 20 aa, Artificial sequence
  A myelin protein unique epitope containing peptide
GLRGEPPELDLSYSHSDLGK

SEQ ID NO: 72 - protein, 20 aa, Artificial sequence
  A myelin protein unique epitope containing peptide
DLSYSHSDLGKRPTKDSYTL

SEQ ID NO: 73 - protein, 20 aa, Artificial sequence
  A myelin protein unique epitope containing peptide
GKRPTKDSYTLTEELAECCQ

SEQ ID NO: 74 - protein, 20 aa, Artificial sequence
  A myelin protein unique epitope containing peptide
DIVNTPNIVVPLEVVAGTEV

SEQ ID NO: 75 - protein, 20 aa, Artificial sequence
  A myelin protein unique epitope containing peptide
MDVKYPPVIVEVNSSVEAIE

SEQ ID NO: 76 - protein, 20 aa, Artificial sequence
  A myelin protein unique epitope containing peptide
LELPFQGAHRLTWAKIGPVG

SEQ ID NO: 77 - protein, 20 aa, Artificial sequence
  A myelin protein unique epitope containing peptide
NRTVGLSVMYASWKPTVNGT

SEQ ID NO: 78 - protein, 20 aa, Artificial sequence
  A myelin protein unique epitope containing peptide
DLSYSHSDLGKQPTKDSYTL

SEQ ID NO: 79 - protein, 20 aa, Artificial sequence
  A myelin protein unique epitope containing peptide
RISGAPEKYESKEVSTLESH

SEQ ID NO: 80 - protein, 20 aa, Artificial sequence
  A myelin protein unique epitope containing peptide
MEYQILKMSLCLFILLFLTP

SEQ ID NO: 81 - protein, 20 aa, Artificial sequence
  A myelin protein unique epitope containing peptide
LCLFILLFLTPGILCICPLQ

SEQ ID NO: 82 - protein, 20 aa, Artificial sequence
  A myelin protein unique epitope containing peptide
TPGILCICPLQCICTERHRH

SEQ ID NO: 83 - protein, 20 aa, Artificial sequence
  A myelin protein unique epitope containing peptide
LQCICTERHRHVDCSGRNLS

SEQ ID NO: 84 - protein, 20 aa, Artificial sequence
  A myelin protein unique epitope containing peptide
RHVDCSGRNLSTLPSGLQEN

SEQ ID NO: 85 - protein, 20 aa, Artificial sequence
  A myelin protein unique epitope containing peptide
LSTLPSGLQENIIHLNLSYN

SEQ ID NO: 86 - protein, 20 aa, Artificial sequence
  A myelin protein unique epitope containing peptide
ENIIHLNLSYNHFTDLHNQL

SEQ ID NO: 87 - protein, 20 aa, Artificial sequence
  A myelin protein unique epitope containing peptide
YNHFTDLHNQLTQYTNLRTL

SEQ ID NO: 88 - protein, 20 aa, Artificial sequence
  A myelin protein unique epitope containing peptide
QLTQYTNLRTLDISNNRLES

SEQ ID NO: 89 - protein, 20 aa, Artificial sequence
  A myelin protein unique epitope containing peptide
TLDISNNRLESLPAHLPRSL

SEQ ID NO: 90 - protein, 20 aa, Artificial sequence
  A myelin protein unique epitope containing peptide
ESLPAHLPRSLWNMSAANNN

SEQ ID NO: 91 - protein, 20 aa, Artificial sequence
  A myelin protein unique epitope containing peptide
SLWNMSAANNNIKLLDKSDT

SEQ ID NO: 92 - protein, 20 aa, Artificial sequence
  A myelin protein unique epitope containing peptide
NNIKLLDKSDTAYQWNLKYL

SEQ ID NO: 93 - protein, 20 aa, Artificial sequence
  A myelin protein unique epitope containing peptide
DTAYQWNLKYLDVSKNMLEK

SEQ ID NO: 94 - protein, 20 aa, Artificial sequence
  A myelin protein unique epitope containing peptide
YLDVSKNMLEKVVLIKNTLR

SEQ ID NO: 95 - protein, 20 aa, Artificial sequence
  A myelin protein unique epitope containing peptide
EKVVLIKNTLRSLEVLNLSS

SEQ ID NO: 96 - protein, 20 aa, Artificial sequence
  A myelin protein unique epitope containing peptide
LRSLEVLNLSSNKLWTVPTN

SEQ ID NO: 97 - protein, 20 aa, Artificial sequence
  A myelin protein unique epitope containing peptide
SSNKLWTVPTNMPSKLHIVD

SEQ ID NO: 98 - protein, 20 aa, Artificial sequence
  A myelin protein unique epitope containing peptide
TNMPSKLHIVDLSNNSLTQI

SEQ ID NO: 99 - protein, 20 aa, Artificial sequence
  A myelin protein unique epitope containing peptide
VDLSNNSLTQILPGTLINLT

SEQ ID NO: 100 - protein, 20 aa, Artificial sequence
  A myelin protein unique epitope containing peptide
QILPGTLINLTNLTHLYLHN

SEQ ID NO: 101 - protein, 20 aa, Artificial sequence
  A myelin protein unique epitope containing peptide
LTNLTHLYLHNNKFTFIPDQ

SEQ ID NO: 102 - protein, 20 aa, Artificial sequence
  A myelin protein unique epitope containing peptide
HNNKFTFIPDQSFDQLFQLQ

SEQ ID NO: 103 - protein, 20 aa, Artificial sequence
  A myelin protein unique epitope containing peptide
DQSFDQLFQLQEITLYNNRW

SEQ ID NO: 104 - protein, 20 aa, Artificial sequence
  A myelin protein unique epitope containing peptide
LQEITLYNNRWSCDHKQNIT

SEQ ID NO: 105 - protein, 20 aa, Artificial sequence
  A myelin protein unique epitope containing peptide
RWSCDHKQNITYLLKWMMET

SEQ ID NO: 106 - protein, 20 aa, Artificial sequence
  A myelin protein unique epitope containing peptide
ITYLLKWMMETKAHVIGTPC

SEQ ID NO: 107 - protein, 20 aa, Artificial sequence
  A myelin protein unique epitope containing peptide
ETKAHVIGTPCSTQISSLKE

SEQ ID NO: 108 - protein, 20 aa, Artificial sequence
  A myelin protein unique epitope containing peptide
PCSTQISSLKEHNMYPTPSG

SEQ ID NO: 109 - protein, 20 aa, Artificial sequence
  A myelin protein unique epitope containing peptide
KEHNMYPTPSGFTSSLFTVS

SEQ ID NO: 110 - protein, 20 aa, Artificial sequence
  A myelin protein unique epitope containing peptide
SGFTSSLFTVSGMQTVDTIN

SEQ ID NO: 111 - protein, 20 aa, Artificial sequence
  A myelin protein unique epitope containing peptide
VSGMQTVDTINSLSVVTQPK

SEQ ID NO: 112 - protein, 20 aa, Artificial sequence
  A myelin protein unique epitope containing peptide
INSLSVVTQPKVTKIPKQYR

SEQ ID NO: 113 - protein, 20 aa, Artificial sequence
  A myelin protein unique epitope containing peptide
PKVTKIPKQYRTKETTFGAT

SEQ ID NO: 114 - protein, 20 aa, Artificial sequence
  A myelin protein unique epitope containing peptide
YRTKETTFGATLSKDTTFTS

SEQ ID NO: 115 - protein, 20 aa, Artificial sequence
  A myelin protein unique epitope containing peptide
ATLSKDTTFTSTDKAFVPYP

SEQ ID NO: 116 - protein, 20 aa, Artificial sequence
  A myelin protein unique epitope containing peptide
TSTDKAFVPYPEDTSTETIN

SEQ ID NO: 117 - protein, 20 aa, Artificial sequence
  A myelin protein unique epitope containing peptide
YPEDTSTETINSHEAAAATL

SEQ ID NO: 118 - protein, 20 aa, Artificial sequence
  A myelin protein unique epitope containing peptide
INSHEAAAATLTIHLQDGMV

SEQ ID NO: 119 - protein, 20 aa, Artificial sequence
  A myelin protein unique epitope containing peptide
TLTIHLQDGMVTNTSLTSST

SEQ ID NO: 120 - protein, 20 aa, Artificial sequence
  A myelin protein unique epitope containing peptide
MVTNTSLTSSTKSSPTPMTL

SEQ ID NO: 121 - protein, 20 aa, Artificial sequence
  A myelin protein unique epitope containing peptide
STKSSPTPMTLSITSGMPNN

SEQ ID NO: 122 - protein, 20 aa, Artificial sequence
  A myelin protein unique epitope containing peptide
TLSITSGMPNNFSEMPQQST

SEQ ID NO: 123 - protein, 20 aa, Artificial sequence
  A myelin protein unique epitope containing peptide
NNFSEMPQQSTTLNLWREET

SEQ ID NO: 124 - protein, 20 aa, Artificial sequence
  A myelin protein unique epitope containing peptide
STTLNLWREETTTNVKTPLP

SEQ ID NO: 125 - protein, 20 aa, Artificial sequence
  A myelin protein unique epitope containing peptide
ETTTNVKTPLPSVANAWKVN

SEQ ID NO: 126 - protein, 20 aa, Artificial sequence
  A myelin protein unique epitope containing peptide
LPSVANAWKVNASFLLLLNV

SEQ ID NO: 127 - protein, 20 aa, Artificial sequence
  A myelin protein unique epitope containing peptide
MNRGFSRKSHTFLPKIFFRK

SEQ ID NO: 128 - protein, 20 aa, Artificial sequence
  A myelin protein unique epitope containing peptide
HTFLPKIFFRKMSSSGAKDK

SEQ ID NO: 129 - protein, 20 aa, Artificial sequence
  A myelin protein unique epitope containing peptide
RKMSSSGAKDKPELQFPFLQ

SEQ ID NO: 130 - protein, 20 aa, Artificial sequence
  A myelin protein unique epitope containing peptide
DKPELQFPFLQDEDTVATLL

SEQ ID NO: 131 - protein, 20 aa, Artificial sequence
  A myelin protein unique epitope containing peptide
LQDEDTVATLLECKTLFILR

SEQ ID NO: 132 - protein, 20 aa, Artificial sequence
  A myelin protein unique epitope containing peptide
LLECKTLFILRGLPGSGKST

SEQ ID NO: 133 - protein, 20 aa, Artificial sequence
  A myelin protein unique epitope containing peptide
LRGLPGSGKSTLARVIVDKY

SEQ ID NO: 134 - protein, 20 aa, Artificial sequence
  A myelin protein unique epitope containing peptide
STLARVIVDKYRDGTKMVSA

SEQ ID NO: 135 - protein, 20 aa, Artificial sequence
  A myelin protein unique epitope containing peptide
KYRDGTKMVSADAYKITPGA

SEQ ID NO: 136 - protein, 20 aa, Artificial sequence
  A myelin protein unique epitope containing peptide
SADAYKITPGARGAFSEEYK

SEQ ID NO: 137 - protein, 20 aa, Artificial sequence
  A myelin protein unique epitope containing peptide
GARGAFSEEYKRLDEDLAAY

SEQ ID NO: 138 - protein, 20 aa, Artificial sequence
  A myelin protein unique epitope containing peptide
YKRLDEDLAAYCRRRDIRIL

SEQ ID NO: 139 - protein, 20 aa, Artificial sequence
  A myelin protein unique epitope containing peptide
AYCRRRDIRILVLDDTNHER

SEQ ID NO: 140 - protein, 20 aa, Artificial sequence
  A myelin protein unique epitope containing peptide
ILVLDDTNHERERLEQLFEM

SEQ ID NO: 141 - protein, 20 aa, Artificial sequence
  A myelin protein unique epitope containing peptide
ERERLEQLFEMADQYQYQVV

SEQ ID NO: 142 - protein, 20 aa, Artificial sequence
  A myelin protein unique epitope containing peptide
EMADQYQYQVVLVEPKTAWR

SEQ ID NO: 143 - protein, 20 aa, Artificial sequence
  A myelin protein unique epitope containing peptide
VVLVEPKTAWRLDCAQLKEK

SEQ ID NO: 144 - protein, 20 aa, Artificial sequence
  A myelin protein unique epitope containing peptide
WRLDCAQLKEKNQWQLSADD

SEQ ID NO: 145 - protein, 20 aa, Artificial sequence
  A myelin protein unique epitope containing peptide
EKNQWQLSADDLKKLKPGLE

SEQ ID NO: 146 - protein, 20 aa, Artificial sequence
  A myelin protein unique epitope containing peptide
DDLKKLKPGLEKDFLPLYFG

SEQ ID NO: 147 - protein, 20 aa, Artificial sequence
  A myelin protein unique epitope containing peptide
LEKDFLPLYFGWFLTKKSSE

SEQ ID NO: 148 - protein, 20 aa, Artificial sequence
  A myelin protein unique epitope containing peptide
FGWFLTKKSSETLRKAGQVF

SEQ ID NO: 149 - protein, 20 aa, Artificial sequence
  A myelin protein unique epitope containing peptide
SETLRKAGQVFLEELGNHKA

SEQ ID NO: 150 - protein, 20 aa, Artificial sequence
  A myelin protein unique epitope containing peptide
VFLEELGNHKAFKKELRQFV

SEQ ID NO: 151 - protein, 20 aa, Artificial sequence
  A myelin protein unique epitope containing peptide
KAFKKELRQFVPGDEPREKM

SEQ ID NO: 152 - protein, 20 aa, Artificial sequence
  A myelin protein unique epitope containing peptide
FVPGDEPREKMDLVTYFGKR

SEQ ID NO: 153 - protein, 20 aa, Artificial sequence
  A myelin protein unique epitope containing peptide
KMDLVTYFGKRPPGVLHCTT

SEQ ID NO: 154 - protein, 20 aa, Artificial sequence
  A myelin protein unique epitope containing peptide
KRPPGVLHCTTKFCDYGKAP

SEQ ID NO: 155 - protein, 20 aa, Artificial sequence
  A myelin protein unique epitope containing peptide
TTKFCDYGKAPGAEEYAQQD

SEQ ID NO: 156 - protein, 20 aa, Artificial sequence
  A myelin protein unique epitope containing peptide
APGAEEYAQQDVLKKSYSKA

SEQ ID NO: 157 - protein, 20 aa, Artificial sequence
  A myelin protein unique epitope containing peptide
QDVLKKSYSKAFTLTISALF

SEQ ID NO: 158 - protein, 20 aa, Artificial sequence
  A myelin protein unique epitope containing peptide
KAFTLTISALFVTPKTTGAR

SEQ ID NO: 159 - protein, 20 aa, Artificial sequence
  A myelin protein unique epitope containing peptide
LFVTPKTTGARVELSEQQLQ

SEQ ID NO: 160 - protein, 20 aa, Artificial sequence
  A myelin protein unique epitope containing peptide
ARVELSEQQLQLWPSDVDKL

SEQ ID NO: 161 - protein, 20 aa, Artificial sequence
  A myelin protein unique epitope containing peptide
LQLWPSDVDKLSPTDNLPRG

SEQ ID NO: 162 - protein, 20 aa, Artificial sequence
  A myelin protein unique epitope containing peptide
KLSPTDNLPRGSRAHITLGC

SEQ ID NO: 163 - protein, 20 aa, Artificial sequence
  A myelin protein unique epitope containing peptide
RGSRAHITLGCAADVEAVQT

SEQ ID NO: 164 - protein, 20 aa, Artificial sequence
  A myelin protein unique epitope containing peptide
GCAADVEAVQTGLDLLEILR

SEQ ID NO: 165 - protein, 20 aa, Artificial sequence
  A myelin protein unique epitope containing peptide
QTGLDLLEILRQEKGGSRGE

SEQ ID NO: 166 - protein, 20 aa, Artificial sequence
  A myelin protein unique epitope containing peptide
LRQEKGGSRGEEVGELSRGK

SEQ ID NO: 167 - protein, 20 aa, Artificial sequence
  A myelin protein unique epitope containing peptide
GEEVGELSRGKLYSLGNGRW

SEQ ID NO: 168 - protein, 20 aa, Artificial sequence
  A myelin protein unique epitope containing peptide
GKLYSLGNGRWMLTLAKNME

SEQ ID NO: 169 - protein, 20 aa, Artificial sequence
  A myelin protein unique epitope containing peptide
RWMLTLAKNMEVRAIFTGYY

SEQ ID NO: 170 - protein, 20 aa, Artificial sequence
  A myelin protein unique epitope containing peptide
MEVRAIFTGYYGKGKPVPTQ

SEQ ID NO: 171 - protein, 20 aa, Artificial sequence
  A myelin protein unique epitope containing peptide
YYGKGKPVPTQGSRKGGALQ

SEQ ID NO: 172 - protein, 20 aa, Artificial sequence
  A myelin protein unique epitope containing peptide
MASLSRPSLPSCLCSFLLLL

SEQ ID NO: 173 - protein, 20 aa, Artificial sequence
  A myelin protein unique epitope containing peptide
PSCLCSFLLLLLLQVSSSYA

SEQ ID NO: 174 - protein, 20 aa, Artificial sequence
  A myelin protein unique epitope containing peptide
LLLLQVSSSYAGQFRVIGPR

SEQ ID NO: 175 - protein, 20 aa, Artificial sequence
  A myelin protein unique epitope containing peptide
YAGQFRVIGPRHPIRALVGD

SEQ ID NO: 176 - protein, 20 aa, Artificial sequence
  A myelin protein unique epitope containing peptide
PRHPIRALVGDEVELPCRIS

SEQ ID NO: 177 - protein, 20 aa, Artificial sequence
  A myelin protein unique epitope containing peptide
GDEVELPCRISPGKNATGME

SEQ ID NO: 178 - protein, 20 aa, Artificial sequence
  A myelin protein unique epitope containing peptide
ISPGKNATGMEVGWYRPPFS

SEQ ID NO: 179 - protein, 20 aa, Artificial sequence
  A myelin protein unique epitope containing peptide
MEVGWYRPPFSRVVHLYRNG

SEQ ID NO: 180 - protein, 20 aa, Artificial sequence
  A myelin protein unique epitope containing peptide
FSRVVHLYRNGKDQDGDQAP

SEQ ID NO: 181 - protein, 20 aa, Artificial sequence
  A myelin protein unique epitope containing peptide
NGKDQDGDQAPEYRGRTELL

SEQ ID NO: 182 - protein, 20 aa, Artificial sequence
  A myelin protein unique epitope containing peptide
APEYRGRTELLKDAIGEGKV

SEQ ID NO: 183 - protein, 20 aa, Artificial sequence
  A myelin protein unique epitope containing peptide
LLKDAIGEGKVTLRIRNVRF

SEQ ID NO: 184 - protein, 20 aa, Artificial sequence
  A myelin protein unique epitope containing peptide
KVTLRIRNVRFSDEGGFTCF

SEQ ID NO: 185 - protein, 20 aa, Artificial sequence
  A myelin protein unique epitope containing peptide
RFSDEGGFTCFFRDHSYQEE

SEQ ID NO: 186 - protein, 20 aa, Artificial sequence
  A myelin protein unique epitope containing peptide
CFFRDHSYQEEAAMELKVED

SEQ ID NO: 187 - protein, 20 aa, Artificial sequence
  A myelin protein unique epitope containing peptide
EEAAMELKVEDPFYWVSPGV

SEQ ID NO: 188 - protein, 20 aa, Artificial sequence
  A myelin protein unique epitope containing peptide
EDPFYWVSPGVLVLLAVLPV

SEQ ID NO: 189 - protein, 20 aa, Artificial sequence
  A myelin protein unique epitope containing peptide
GVLVLLAVLPVLLLQITVGL

SEQ ID NO: 190 - protein, 20 aa, Artificial sequence
  A myelin protein unique epitope containing peptide
PVLLLQITVGLVFLCLQYRL

SEQ ID NO: 191 - protein, 20 aa, Artificial sequence
  A myelin protein unique epitope containing peptide
GLVFLCLQYRLRGKLRAEIE

SEQ ID NO: 192 - protein, 20 aa, Artificial sequence
  A myelin protein unique epitope containing peptide
RLRGKLRAEIENLHRTFDPH

SEQ ID NO: 193 - protein, 20 aa, Artificial sequence
  A myelin protein unique epitope containing peptide
IENLHRTFDPHFLRVPCWKI

SEQ ID NO: 194 - protein, 20 aa, Artificial sequence
  A myelin protein unique epitope containing peptide
PHFLRVPCWKITLFVIVPVL

SEQ ID NO: 195 - protein, 20 aa, Artificial sequence
  A myelin protein unique epitope containing peptide
KITLFVIVPVLGPLVALIIC

SEQ ID NO: 196 - protein, 20 aa, Artificial sequence
  A myelin protein unique epitope containing peptide
VLGPLVALIICYNWLHRRLA

SEQ ID NO: 197 - protein, 20 aa, Artificial sequence
  A myelin protein unique epitope containing peptide
ICYNWLHRRLAGQFLEELPS

SEQ ID NO: 198 - protein, 20 aa, Artificial sequence
  A myelin protein unique epitope containing peptide
LAGQFLEELPSRIDFPSSVI

SEQ ID NO: 199 - protein, 20 aa, Artificial sequence
  A myelin protein unique epitope containing peptide
PSRIDFPSSVIYLFSSFSHF

SEQ ID NO: 200 - protein, 20 aa, Artificial sequence
  A myelin protein unique epitope containing peptide
VIYLFSSFSHFYYPSFHFSL

SEQ ID NO: 201 - protein, 20 aa, Artificial sequence
  A myelin protein unique epitope containing peptide
HFYYPSFHFSLQSSTWKPSL

SEQ ID NO: 202 - protein, 20 aa, Artificial sequence
  A myelin protein unique epitope containing peptide
SLQSSTWKPSLAKDRQVPLS

SEQ ID NO: 203 - protein, 20 aa, Artificial sequence
  A myelin protein unique epitope containing peptide
SLAKDRQVPLSPSEDTCTGE

SEQ ID NO: 204 - protein, 20 aa, Artificial sequence
  A myelin protein unique epitope containing peptide
LSPSEDTCTGEQHRMVSAMN

SEQ ID NO: 205 - protein, 20 aa, Artificial sequence
  A myelin protein unique epitope containing peptide
GEQHRMVSAMNWRPGISSLK

SEQ ID NO: 206 - protein, 20 aa, Artificial sequence
  A myelin protein unique epitope containing peptide
MGNHAGKRELNAEKASTNSE

SEQ ID NO: 207 - protein, 20 aa, Artificial sequence
  A myelin protein unique epitope containing peptide
LNAEKASTNSETNRGESEKK

SEQ ID NO: 208 - protein, 20 aa, Artificial sequence
  A myelin protein unique epitope containing peptide
SETNRGESEKKRNLGELSRT

SEQ ID NO: 209 - protein, 20 aa, Artificial sequence
  A myelin protein unique epitope containing peptide
KKRNLGELSRTTSEDNEVFG

SEQ ID NO: 210 - protein, 20 aa, Artificial sequence
  A myelin protein unique epitope containing peptide
RTTSEDNEVFGEADANQNNG

SEQ ID NO: 211 - protein, 20 aa, Artificial sequence
  A myelin protein unique epitope containing peptide
FGEADANQNNGTSSQDTAVT

SEQ ID NO: 212 - protein, 20 aa, Artificial sequence
  A myelin protein unique epitope containing peptide
NGTSSQDTAVTDSKRTADPK

SEQ ID NO: 213 - protein, 20 aa, Artificial sequence
  A myelin protein unique epitope containing peptide
VTDSKRTADPKNAWQDAHPA

SEQ ID NO: 214 - protein, 20 aa, Artificial sequence
  A myelin protein unique epitope containing peptide
PKNAWQDAHPADPGSRPHLI

SEQ ID NO: 215 - protein, 20 aa, Artificial sequence
  A myelin protein unique epitope containing peptide
PADPGSRPHLIRLFSRDAPG

SEQ ID NO: 216 - protein, 20 aa, Artificial sequence
  A myelin protein unique epitope containing peptide
LIRLFSRDAPGREDNTFKDR

SEQ ID NO: 217 - protein, 20 aa, Artificial sequence
  A myelin protein unique epitope containing peptide
PGREDNTFKDRPSESDELQT

SEQ ID NO: 218 - protein, 20 aa, Artificial sequence
  A myelin protein unique epitope containing peptide
DRPSESDELQTIQEDSAATS

SEQ ID NO: 219 - protein, 20 aa, Artificial sequence
  A myelin protein unique epitope containing peptide
QTIQEDSAATSESLDVMASQ

SEQ ID NO: 220 - protein, 20 aa, Artificial sequence
  A myelin protein unique epitope containing peptide
TSESLDVMASQKRPSQRHGS

SEQ ID NO: 221 - protein, 20 aa, Artificial sequence
  A myelin protein unique epitope containing peptide
SQKRPSQRHGSKYLATASTM

SEQ ID NO: 222 - protein, 20 aa, Artificial sequence
  A myelin protein unique epitope containing peptide
GSKYLATASTMDHARHGFLP

SEQ ID NO: 223 - protein, 20 aa, Artificial sequence
  A myelin protein unique epitope containing peptide
TMDHARHGFLPRHRDTGILD

SEQ ID NO: 224 - protein, 20 aa, Artificial sequence
  A myelin protein unique epitope containing peptide
LPRHRDTGILDSIGRFFGGD

SEQ ID NO: 225 - protein, 20 aa, Artificial sequence
  A myelin protein unique epitope containing peptide
LDSIGRFFGGDRGAPKRGSG

SEQ ID NO: 226 - protein, 20 aa, Artificial sequence
  A myelin protein unique epitope containing peptide
GDRGAPKRGSGKDSHHPART

SEQ ID NO: 227 - protein, 20 aa, Artificial sequence
  A myelin protein unique epitope containing peptide
SGKDSHHPARTAHYGSLPQK

SEQ ID NO: 228 - protein, 20 aa, Artificial sequence
  A myelin protein unique epitope containing peptide
RTAHYGSLPQKSHGRTQDEN

SEQ ID NO: 229 - protein, 20 aa, Artificial sequence
  A myelin protein unique epitope containing peptide
QKSHGRTQDENPVVHFFKNI

SEQ ID NO: 230 - protein, 20 aa, Artificial sequence
  A myelin protein unique epitope containing peptide
ENPVVHFFKNIVTPRTPPPS

SEQ ID NO: 231 - protein, 20 aa, Artificial sequence
  A myelin protein unique epitope containing peptide
NIVTPRTPPPSQGKGRGLSL

SEQ ID NO: 232 - protein, 20 aa, Artificial sequence
  A myelin protein unique epitope containing peptide
PSQGKGRGLSLSRFSWGAEG

SEQ ID NO: 233 - protein, 20 aa, Artificial sequence
  A myelin protein unique epitope containing peptide
SLSRFSWGAEGQRPGFGYGG

SEQ ID NO: 234 - protein, 20 aa, Artificial sequence
  A myelin protein unique epitope containing peptide
EGQRPGFGYGGRASDYKSAH

SEQ ID NO: 235 - protein, 20 aa, Artificial sequence
  A myelin protein unique epitope containing peptide
GGRASDYKSAHKGFKGVDAQ

SEQ ID NO: 236 - protein, 20 aa, Artificial sequence
  A myelin protein unique epitope containing peptide
AHKGFKGVDAQGTLSKIFKL

SEQ ID NO: 237 - protein, 20 aa, Artificial sequence
  A myelin protein unique epitope containing peptide
AQGTLSKIFKLGGRDSRSGS

SEQ ID NO: 238 - protein, 20 aa, Artificial sequence
  A myelin protein unique epitope containing peptide
RAEIENLHRTFESFGVLGPQ

SEQ ID NO: 239 - protein, 20 aa, Artificial sequence
  A myelin protein unique epitope containing peptide
TFESFGVLGPQVKEPKKTGQ

SEQ ID NO: 240 - protein, 20 aa, Artificial sequence
  A myelin protein unique epitope containing peptide
PQVKEPKKTGQFLEELPSRI

SEQ ID NO: 241 - protein, 20 aa, Artificial sequence
  A myelin protein unique epitope containing peptide
RRLAGQFLEELRKFSSLCYK

SEQ ID NO: 242 - protein, 20 aa, Artificial sequence
  A myelin protein unique epitope containing peptide
ELRKFSSLCYKQRIKSQERE

SEQ ID NO: 243 - protein, 20 aa, Artificial sequence
  A myelin protein unique epitope containing peptide
YKQRIKSQERETEATRGRGG

SEQ ID NO: 244 - protein, 20 aa, Artificial sequence
  A myelin protein unique epitope containing peptide
RETEATRGRGGLLRDHIPRG

SEQ ID NO: 245 - protein, 20 aa, Artificial sequence
  A myelin protein unique epitope containing peptide
GGLLRDHIPRGKEELESLGG

SEQ ID NO: 246 - protein, 20 aa, Artificial sequence
  A myelin protein unique epitope containing peptide
RAEIENLHRTFGQFLEELPS

SEQ ID NO: 247 - protein, 20 aa, Artificial sequence
  A myelin protein unique epitope containing peptide
MGLLECCARCLVGAPFASLV

SEQ ID NO: 248 - protein, 20 aa, Artificial sequence
  A myelin protein unique epitope containing peptide
CLVGAPFASLVATGLCFFGV

SEQ ID NO: 249 - protein, 20 aa, Artificial sequence
  A myelin protein unique epitope containing peptide
LVATGLCFFGVALFCGCGHE

SEQ ID NO: 250 - protein, 20 aa, Artificial sequence
  A myelin protein unique epitope containing peptide
GVALFCGCGHEALTGTEKLI

SEQ ID NO: 251 - protein, 20 aa, Artificial sequence
  A myelin protein unique epitope containing peptide
HEALTGTEKLIETYFSKNYQ

SEQ ID NO: 252 - protein, 20 aa, Artificial sequence
  A myelin protein unique epitope containing peptide
LIETYFSKNYQDYEYLINVI

SEQ ID NO: 253 - protein, 20 aa, Artificial sequence
  A myelin protein unique epitope containing peptide
YQDYEYLINVIHAFQYVIYG

SEQ ID NO: 254 - protein, 20 aa, Artificial sequence
  A myelin protein unique epitope containing peptide
VIHAFQYVIYGTASFFFLYG

SEQ ID NO: 255 - protein, 20 aa, Artificial sequence
  A myelin protein unique epitope containing peptide
YGTASFFFLYGALLLAEGFY

SEQ ID NO: 256 - protein, 20 aa, Artificial sequence
  A myelin protein unique epitope containing peptide
YGALLLAEGFYTTGAVRQIF

SEQ ID NO: 257 - protein, 20 aa, Artificial sequence
  A myelin protein unique epitope containing peptide
FYTTGAVRQIFGDYKTTICG

SEQ ID NO: 258 - protein, 20 aa, Artificial sequence
  A myelin protein unique epitope containing peptide
IFGDYKTTICGKGLSATVTG

SEQ ID NO: 259 - protein, 20 aa, Artificial sequence
  A myelin protein unique epitope containing peptide
CGKGLSATVTGGQKGRGSRG

SEQ ID NO: 260 - protein, 20 aa, Artificial sequence
  A myelin protein unique epitope containing peptide
TGGQKGRGSRGQHQAHSLER

SEQ ID NO: 261 - protein, 20 aa, Artificial sequence
  A myelin protein unique epitope containing peptide
RGQHQAHSLERVCHCLGKWL

SEQ ID NO: 262 - protein, 20 aa, Artificial sequence
  A myelin protein unique epitope containing peptide
ERVCHCLGKWLGHPDKFVGI

SEQ ID NO: 263 - protein, 20 aa, Artificial sequence
  A myelin protein unique epitope containing peptide
WLGHPDKFVGITYALTVVWL

SEQ ID NO: 264 - protein, 20 aa, Artificial sequence
  A myelin protein unique epitope containing peptide
GITYALTVVWLLVFACSAVP

SEQ ID NO: 265 - protein, 20 aa, Artificial sequence
  A myelin protein unique epitope containing peptide
WLLVFACSAVPVYIYFNTWT

SEQ ID NO: 266 - protein, 20 aa, Artificial sequence
  A myelin protein unique epitope containing peptide
VPVYIYFNTWTTCQSIAFPS

SEQ ID NO: 267 - protein, 20 aa, Artificial sequence
  A myelin protein unique epitope containing peptide
WTTCQSIAFPSKTSASIGSL

SEQ ID NO: 268 - protein, 20 aa, Artificial sequence
  A myelin protein unique epitope containing peptide
PSKTSASIGSLCADARMYGV

SEQ ID NO: 269 - protein, 20 aa, Artificial sequence
  A myelin protein unique epitope containing peptide
SLCADARMYGVLPWNAFPGK

SEQ ID NO: 270 - protein, 20 aa, Artificial sequence
  A myelin protein unique epitope containing peptide
GVLPWNAFPGKVCGSNLLSI

SEQ ID NO: 271 - protein, 20 aa, Artificial sequence
  A myelin protein unique epitope containing peptide
GKVCGSNLLSICKTAEFQMT

SEQ ID NO: 272 - protein, 20 aa, Artificial sequence
  A myelin protein unique epitope containing peptide
SICKTAEFQMTFHLFIAAFV

SEQ ID NO: 273 - protein, 20 aa, Artificial sequence
  A myelin protein unique epitope containing peptide
MTFHLFIAAFVGAAATLVSL

SEQ ID NO: 274 - protein, 20 aa, Artificial sequence
  A myelin protein unique epitope containing peptide
FVGAAATLVSLLTFMIAATY

SEQ ID NO: 275 - protein, 20 aa, Artificial sequence
  A myelin protein unique epitope containing peptide
SLLTFMIAATYNFAVLKLMG

SEQ ID NO: 276 - protein, 20 aa, Artificial sequence
  A myelin protein unique epitope containing peptide
VYIYFNTWTTCDSIAFPSKT

SEQ ID NO: 277 - protein, 20 aa, Artificial sequence
  A myelin protein unique epitope containing peptide
ADARMYGVLPWIAFPGKVCG

SEQ ID NO: 278 - protein, 20 aa, Artificial sequence
  A myelin protein unique epitope containing peptide
QHQAHSLERVCTCLGKWLGH

SEQ ID NO: 279 - protein, 20 aa, Artificial sequence
  A myelin protein unique epitope containing peptide
PCCGGSLSPGTVEMASLSRP

SEQ ID NO: 280 - protein, 20 aa, Artificial sequence
  A myelin protein unique epitope containing peptide
PVLLLQITVGLIFLCLQYRL

SEQ ID NO: 281 - protein, 20 aa, Artificial sequence
  A myelin protein unique epitope containing peptide
RRLAGQFLEELLFHLEALSG

SEQ ID NO: 282 - protein, 20 aa, Artificial sequence
  A myelin protein unique epitope containing peptide
AVLPVLLLQITLGLVFLCLQ

SEQ ID NO: 283 - protein, 20 aa, Artificial sequence
  A myelin protein unique epitope containing peptide
TTICGKGLSATFVGITYALT

SEQ ID NO: 284 - protein, 20 aa, Artificial sequence
  A myelin protein unique epitope containing peptide
APFASLVATGLGFFGVALFC

SEQ ID NO: 285 - protein, 20 aa, Artificial sequence
  A myelin protein unique epitope containing peptide
TFMIAATYNFAILKLMGRGT

SEQ ID NO: 286 - protein, 20 aa, Artificial sequence
  A myelin protein unique epitope containing peptide
MVATCLQVVGFVTSFVGWIG

SEQ ID NO: 287 - protein, 20 aa, Artificial sequence
  A myelin protein unique epitope containing peptide
GFVTSFVGWIGVIVTTSTND

SEQ ID NO: 288 - protein, 20 aa, Artificial sequence
  A myelin protein unique epitope containing peptide
IGVIVTTSTNDWVVTCGYTI

SEQ ID NO: 289 - protein, 20 aa, Artificial sequence
  A myelin protein unique epitope containing peptide
NDWVVTCGYTIPTCRKLDEL

SEQ ID NO: 290 - protein, 20 aa, Artificial sequence
  A myelin protein unique epitope containing peptide
TIPTCRKLDELGSKGLWADC

SEQ ID NO: 291 - protein, 20 aa, Artificial sequence
  A myelin protein unique epitope containing peptide
ELGSKGLWADCVMATGLYHC

SEQ ID NO: 292 - protein, 20 aa, Artificial sequence
  A myelin protein unique epitope containing peptide
DCVMATGLYHCKPLVDILIL

SEQ ID NO: 293 - protein, 20 aa, Artificial sequence
  A myelin protein unique epitope containing peptide
HCKPLVDILILPGYVQACRA

SEQ ID NO: 294 - protein, 20 aa, Artificial sequence
  A myelin protein unique epitope containing peptide
ILPGYVQACRALMIAASVLG

SEQ ID NO: 295 - protein, 20 aa, Artificial sequence
  A myelin protein unique epitope containing peptide
RALMIAASVLGLPAILLLLT

SEQ ID NO: 296 - protein, 20 aa, Artificial sequence
  A myelin protein unique epitope containing peptide
LGLPAILLLLTVLPCIRMGQ

SEQ ID NO: 297 - protein, 20 aa, Artificial sequence
  A myelin protein unique epitope containing peptide
LTVLPCIRMGQEPGVAKYRR

SEQ ID NO: 298 - protein, 20 aa, Artificial sequence
  A myelin protein unique epitope containing peptide
GQEPGVAKYRRAQLAGVLLI

SEQ ID NO: 299 - protein, 20 aa, Artificial sequence
  A myelin protein unique epitope containing peptide
RRAQLAGVLLILLALCALVA

SEQ ID NO: 300 - protein, 20 aa, Artificial sequence
  A myelin protein unique epitope containing peptide
LILLALCALVATIWFPVCAH

SEQ ID NO: 301 - protein, 20 aa, Artificial sequence
  A myelin protein unique epitope containing peptide
VATIWFPVCAHRETTIVSFG

SEQ ID NO: 302 - protein, 20 aa, Artificial sequence
  A myelin protein unique epitope containing peptide
AHRETTIVSFGYSLYAGWIG

SEQ ID NO: 303 - protein, 20 aa, Artificial sequence
  A myelin protein unique epitope containing peptide
FGYSLYAGWIGAVLCLVGGC

SEQ ID NO: 304 - protein, 20 aa, Artificial sequence
  A myelin protein unique epitope containing peptide
IGAVLCLVGGCVILCCAGDA

SEQ ID NO: 305 - protein, 20 aa, Artificial sequence
  A myelin protein unique epitope containing peptide
GCVILCCAGDAQAFGENVST

SEQ ID NO: 306 - protein, 20 aa, Artificial sequence
  A myelin protein unique epitope containing peptide
DAQAFGENVSTTLRALAPRL

SEQ ID NO: 307 - protein, 20 aa, Artificial sequence
  A myelin protein unique epitope containing peptide
STTLRALAPRLMRRVPTYKR

SEQ ID NO: 308 - protein, 20 aa, Artificial sequence
  A myelin protein unique epitope containing peptide
RLMRRVPTYKRAARLPTEVL

SEQ ID NO: 309 - protein, 20 aa, Artificial sequence
  A myelin protein unique epitope containing peptide
RAEIENLHRTFVFHLEALSG

SEQ ID NO: 310 - protein, 20 aa, Artificial sequence
  A myelin protein unique epitope containing peptide
LLLLQVSSSYADPFYWVSPG

SEQ ID NO: 311 - protein, 20 aa, Artificial sequence
  A myelin protein unique epitope containing peptide
DRGAPKRGSGKVPWLKPGRS

SEQ ID NO: 312 - protein, 20 aa, Artificial sequence
  A myelin protein unique epitope containing peptide
GKVPWLKPGRSPLPSHARSQ

SEQ ID NO: 313 - protein, 20 aa, Artificial sequence
  A myelin protein unique epitope containing peptide
RSPLPSHARSQPGLCNMYKD

SEQ ID NO: 314 - protein, 20 aa, Artificial sequence
  A myelin protein unique epitope containing peptide
SQPGLCNMYKDSHHPARTAH

SEQ ID NO: 315 - protein, 20 aa, Artificial sequence
  A myelin protein unique epitope containing peptide
KLGGRDSRSGSPMARRHHHH

SEQ ID NO: 316 - protein, 20 aa, Artificial sequence
  A myelin protein unique epitope containing peptide
MDIAIHHPWIRRPFFPFHSP

SEQ ID NO: 317 - protein, 20 aa, Artificial sequence
  A myelin protein unique epitope containing peptide
IRRPFFPFHSPSRLFDQFFG

SEQ ID NO: 318 - protein, 20 aa, Artificial sequence
  A myelin protein unique epitope containing peptide
SPSRLFDQFFGEHLLESDLF

SEQ ID NO: 319 - protein, 20 aa, Artificial sequence
  A myelin protein unique epitope containing peptide
FGEHLLESDLFPTSTSLSPF

SEQ ID NO: 320 - protein, 20 aa, Artificial sequence
  A myelin protein unique epitope containing peptide
LFPTSTSLSPFYLRPPSFLR

SEQ ID NO: 321 - protein, 20 aa, Artificial sequence
  A myelin protein unique epitope containing peptide
PFYLRPPSFLRAPSWFDTGL

SEQ ID NO: 322 - protein, 20 aa, Artificial sequence
  A myelin protein unique epitope containing peptide
LRAPSWFDTGLSEMRLEKDR

SEQ ID NO: 323 - protein, 20 aa, Artificial sequence
  A myelin protein unique epitope containing peptide
GLSEMRLEKDRFSVNLDVKH

SEQ ID NO: 324 - protein, 20 aa, Artificial sequence
  A myelin protein unique epitope containing peptide
DRFSVNLDVKHFSPEELKVK

SEQ ID NO: 325 - protein, 20 aa, Artificial sequence
  A myelin protein unique epitope containing peptide
KHFSPEELKVKVLGDVIEVH

SEQ ID NO: 326 - protein, 20 aa, Artificial sequence
  A myelin protein unique epitope containing peptide
VKVLGDVIEVHGKHEERQDE

SEQ ID NO: 327 - protein, 20 aa, Artificial sequence
  A myelin protein unique epitope containing peptide
VHGKHEERQDEHGFISREFH

SEQ ID NO: 328 - protein, 20 aa, Artificial sequence
  A myelin protein unique epitope containing peptide
DEHGFISREFHRKYRIPADV

SEQ ID NO: 329 - protein, 20 aa, Artificial sequence
  A myelin protein unique epitope containing peptide
FHRKYRIPADVDPLTITSSL

SEQ ID NO: 330 - protein, 20 aa, Artificial sequence
  A myelin protein unique epitope containing peptide
DVDPLTITSSLSSDGVLTVN

SEQ ID NO: 331 - protein, 20 aa, Artificial sequence
  A myelin protein unique epitope containing peptide
SLSSDGVLTVNGPRKQVSGP

SEQ ID NO: 332 - protein, 20 aa, Artificial sequence
  A myelin protein unique epitope containing peptide
VNGPRKQVSGPERTIPITRE

SEQ ID NO: 333 - protein, 20 aa, Artificial sequence
  A myelin protein unique epitope containing peptide
GPERTIPITREKKPAVTAAP

SEQ ID NO: 334 - protein, 20 aa, Artificial sequence
  A myelin protein unique epitope containing peptide
REKKPAVTAAPKKKMPFLEL

SEQ ID NO: 335 - protein, 20 aa, Artificial sequence
  A myelin protein unique epitope containing peptide
APKKKMPFLELHFLKQESFP

SEQ ID NO: 336 - protein, 20 aa, Artificial sequence
  A myelin protein unique epitope containing peptide
PRTPPPSQGKGAEGQRPGFG

SEQ ID NO: 337 - protein, 20 aa, Artificial sequence
  A myelin protein unique epitope containing peptide
GPERTIPITREEKPAVTAAP

SEQ ID NO: 338 - protein, 20 aa, Artificial sequence
  A myelin protein unique epitope containing peptide
QEEAAMELKVEVSHSVTQDW

SEQ ID NO: 339 - protein, 20 aa, Artificial sequence
  A myelin protein unique epitope containing peptide
VEVSHSVTQDWLQWHDHGSL

SEQ ID NO: 340 - protein, 20 aa, Artificial sequence
  A myelin protein unique epitope containing peptide
GILDSIGRFFGSDRAAPKRG

SEQ ID NO: 341 - protein, 20 aa, Artificial sequence
  A myelin protein unique epitope containing peptide
PKRGSGKDSHHAARTTHYGS

SEQ ID NO: 342 - protein, 20 aa, Artificial sequence
  A myelin protein unique epitope containing peptide
HHAARTTHYGSLPQKSQRSQ

SEQ ID NO: 343 - protein, 20 aa, Artificial sequence
  A myelin protein unique epitope containing peptide
GSLPQKSQRSQDENPVVHFF

SEQ ID NO: 344 - protein, 20 aa, Artificial sequence
  A myelin protein unique epitope containing peptide
LSRFSWGAEGQKPGFGYGGR

SEQ ID NO: 345 - protein, 20 aa, Artificial sequence
  A myelin protein unique epitope containing peptide
GQKPGFGYGGRADYKSKGFK

SEQ ID NO: 346 - protein, 20 aa, Artificial sequence
  A myelin protein unique epitope containing peptide
GRADYKSKGFKGAHDAQGTL

SEQ ID NO: 347 - protein, 20 aa, Artificial sequence
  A myelin protein unique epitope containing peptide
MHGFKTPSSQSLQTKLSVHL

SEQ ID NO: 348 - protein, 20 aa, Artificial sequence
  A myelin protein unique epitope containing peptide
QSLQTKLSVHLICLVWQMRW

SEQ ID NO: 349 - protein, 20 aa, Artificial sequence
  A myelin protein unique epitope containing peptide
HLICLVWQMRWHIGGTSPGM

SEQ ID NO: 350 - protein, 20 aa, Artificial sequence
  A myelin protein unique epitope containing peptide
RWHIGGTSPGMDTWLCSLLA

SEQ ID NO: 351 - protein, 20 aa, Artificial sequence
  A myelin protein unique epitope containing peptide
GMDTWLCSLLARVARGVLGS

SEQ ID NO: 352 - protein, 20 aa, Artificial sequence
  A myelin protein unique epitope containing peptide
LARVARGVLGSRRKRSCSCC

SEQ ID NO: 353 - protein, 20 aa, Artificial sequence
  A myelin protein unique epitope containing peptide
GSRRKRSCSCCRWQFPSAAL

SEQ ID NO: 354 - protein, 20 aa, Artificial sequence
  A myelin protein unique epitope containing peptide
CCRWQFPSAALAPLADEKKC

SEQ ID NO: 355 - protein, 20 aa, Artificial sequence
  A myelin protein unique epitope containing peptide
ALAPLADEKKCSFKAKQWSG

SEQ ID NO: 356 - protein, 20 aa, Artificial sequence
  A myelin protein unique epitope containing peptide
KCSFKAKQWSGCTFTHRSAA

SEQ ID NO: 357 - protein, 20 aa, Artificial sequence
  A myelin protein unique epitope containing peptide
SGCTFTHRSAALFCRAVVMP

SEQ ID NO: 358 - protein, 20 aa, Artificial sequence
  A myelin protein unique epitope containing peptide
AALFCRAVVMPGTTPWGLGI

SEQ ID NO: 359 - protein, 20 aa, Artificial sequence
  A myelin protein unique epitope containing peptide
MPGTTPWGLGIRFQFSLTEL

SEQ ID NO: 360 - protein, 20 aa, Artificial sequence
  A myelin protein unique epitope containing peptide
GIRFQFSLTELEIQLAFAEG

SEQ ID NO: 361 - protein, 20 aa, Artificial sequence
  A myelin protein unique epitope containing peptide
ELEIQLAFAEGGREKWNEKT

SEQ ID NO: 362 - protein, 20 aa, Artificial sequence
  A myelin protein unique epitope containing peptide
MCGWAAYLMREPPPKPEPSS

SEQ ID NO: 363 - protein, 20 aa, Artificial sequence
  A myelin protein unique epitope containing peptide
REPPPKPEPSSLCGQHPGRP

SEQ ID NO: 364 - protein, 20 aa, Artificial sequence
  A myelin protein unique epitope containing peptide
SSLCGQHPGRPGGAASPAPT

SEQ ID NO: 365 - protein, 20 aa, Artificial sequence
  A myelin protein unique epitope containing peptide
RPGGAASPAPTPSAWCWPVA

SEQ ID NO: 366 - protein, 20 aa, Artificial sequence
  A myelin protein unique epitope containing peptide
PTPSAWCWPVAPEPLTSPVR

SEQ ID NO: 367 - protein, 20 aa, Artificial sequence
  A myelin protein unique epitope containing peptide
VAPEPLTSPVRGLGPSPGPW

SEQ ID NO: 368 - protein, 20 aa, Artificial sequence
  A myelin protein unique epitope containing peptide
VRGLGPSPGPWWQLPVAQAA

SEQ ID NO: 369 - protein, 20 aa, Artificial sequence
  A myelin protein unique epitope containing peptide
PWWQLPVAQAACPAPRVEVE

SEQ ID NO: 370 - protein, 20 aa, Artificial sequence
  A myelin protein unique epitope containing peptide
AACPAPRVEVELRGLLLQGA

SEQ ID NO: 371 - protein, 20 aa, Artificial sequence
  A myelin protein unique epitope containing peptide
VELRGLLLQGAEGQRPGFGY

SEQ ID NO: 372 - protein, 20 aa, Artificial sequence
  A myelin protein unique epitope containing peptide
GRGLSLSRFSWREGGRRPET

SEQ ID NO: 373 - protein, 20 aa, Artificial sequence
  A myelin protein unique epitope containing peptide
SWREGGRRPETRIWLRRQSV

SEQ ID NO: 374 - protein, 20 aa, Artificial sequence
  A myelin protein unique epitope containing peptide
TPRTPPPSQGKREGGRRPET

SEQ ID NO: 375 - protein, 20 aa, Artificial sequence
  A myelin protein unique epitope containing peptide
MSELEKAMVALIDVFHQYSG

SEQ ID NO: 376 - protein, 20 aa, Artificial sequence
  A myelin protein unique epitope containing peptide
ALIDVFHQYSGREGDKHKLK

SEQ ID NO: 377 - protein, 20 aa, Artificial sequence
  A myelin protein unique epitope containing peptide
SGREGDKHKLKKSELKELIN

SEQ ID NO: 378 - protein, 20 aa, Artificial sequence
  A myelin protein unique epitope containing peptide
LKKSELKELINNELSHFLEE

SEQ ID NO: 379 - protein, 20 aa, Artificial sequence
  A myelin protein unique epitope containing peptide
INNELSHFLEEIKEQEVVDK

SEQ ID NO: 380 - protein, 20 aa, Artificial sequence
  A myelin protein unique epitope containing peptide
EEIKEQEVVDKVMETLDNDG

SEQ ID NO: 381 - protein, 20 aa, Artificial sequence
  A myelin protein unique epitope containing peptide
DKVMETLDNDGDGECDFQEF

SEQ ID NO: 382 - protein, 20 aa, Artificial sequence
  A myelin protein unique epitope containing peptide
DGDGECDFQEFMAFVAMVTT

SEQ ID NO: 383 - protein, 20 aa, Artificial sequence
  A myelin protein unique epitope containing peptide
EFMAFVAMVTTACHEFFEHE

SEQ ID NO: 384 - protein, 20 aa, Artificial sequence
  A myelin protein unique epitope containing peptide
TASTMDHARHGSLPQKSHGR

SEQ ID NO: 385 - protein, 20 aa, Artificial sequence
  A myelin protein unique epitope containing peptide
SAYTAVSVLMPECMVFSHGM

SEQ ID NO: 386 - protein, 20 aa, Artificial sequence
  A myelin protein unique epitope containing peptide
MPECMVFSHGMLSLARFVAP

SEQ ID NO: 387 - protein, 20 aa, Artificial sequence
  A myelin protein unique epitope containing peptide
GMLSLARFVAPTFCPSAKQL

SEQ ID NO: 388 - protein, 20 aa, Artificial sequence
  A myelin protein unique epitope containing peptide
LPWNAFPGKVCCSNLLSICK

SEQ ID NO: 389 - protein, 20 aa, Artificial sequence
  A myelin protein unique epitope containing peptide
QDWLQWHDHGSLQPPPPRLK

SEQ ID NO: 390 - protein, 20 aa, Artificial sequence
  A myelin protein unique epitope containing peptide
AEGGREKWNEKTSSPGCHFY

SEQ ID NO: 391 - protein, 20 aa, Artificial sequence
  A myelin protein unique epitope containing peptide
TKDSYTLTEELAEYAEIRVK

SEQ ID NO: 392 - protein, 20 aa, Artificial sequence
  A myelin protein unique epitope containing peptide
IPRGKEELESLGGGKTPPGR

SEQ ID NO: 393 - protein, 20 aa, Artificial sequence
  A myelin protein unique epitope containing peptide
FYLRPPSFLRAPSXFDTGLS

SEQ ID NO: 394 - protein, 20 aa, Artificial sequence
  A myelin protein unique epitope containing peptide
AWKVNASFLLLLNVVVMLAV

SEQ ID NO: 395 - protein, 20 aa, Artificial sequence
  A myelin protein unique epitope containing peptide
SLPSCLCSFLLLLLQVSSSY

SEQ ID NO: 396 - protein, 20 aa, Artificial sequence
  A myelin protein unique epitope containing peptide
RANPFRMGNHAGKRELNAEK

SEQ ID NO: 397 - protein, 20 aa, Artificial sequence
  A myelin protein unique epitope containing peptide
GRFFGSDRAAPKRGSGKDSH

SEQ ID NO: 398 - protein, 20 aa, Artificial sequence
  A myelin protein unique epitope containing peptide
KPVPTQGSRKGGALQSCTII

SEQ ID NO: 399 - protein, 20 aa, Artificial sequence
  A myelin protein unique epitope containing peptide
MIAATYNFAVLKLMGRGTKF

SEQ ID NO: 400 - protein, 20 aa, Artificial sequence
  A myelin protein unique epitope containing peptide
KSKGFKGAHDAQGTLSKIFK

SEQ ID NO: 401 - protein, 20 aa, Artificial sequence
  A myelin protein unique epitope containing peptide
SKIFKLGGRDSRSGSTMARR

SEQ ID NO: 402 - protein, 20 aa, Artificial sequence
  A myelin protein unique epitope containing peptide
RMVSAMNWRPGISSLKITVW

SEQ ID NO: 403 - protein, 20 aa, Artificial sequence
  A myelin protein unique epitope containing peptide
GSLSPGTVEMASLSRPSLPS

SEQ ID NO: 404 - protein, 20 aa, Artificial sequence
  A myelin protein unique epitope containing peptide
PQVKEPKKTGQFLEELLFHL

SEQ ID NO: 405 - protein, 20 aa, Artificial sequence
  A myelin protein unique epitope containing peptide
PQVKEPKKTGQFLEELRNPF

SEQ ID NO: 406 - protein, 20 aa, Artificial sequence
  A myelin protein unique epitope containing peptide
EIENLHRTFGQFLEELLFHL

SEQ ID NO: 407 - protein, 20 aa, Artificial sequence
  A myelin protein unique epitope containing peptide
EIENLHRTFGQFLEELRNPF

SEQ ID NO: 408 - protein, 20 aa, Artificial sequence
  A myelin protein unique epitope containing peptide
FFGGDRGAPKRGSGKVSSEE

SEQ ID NO: 409 - protein, 20 aa, Artificial sequence
  A myelin protein unique epitope containing peptide
NTPNIVVPLEVVAGTEVEVS

SEQ ID NO: 410 - protein, 20 aa, Artificial sequence
  A myelin protein unique epitope containing peptide
KYPPVIVEVNSSVEAIEGSH

SEQ ID NO: 411 - protein, 20 aa, Artificial sequence
  A myelin protein unique epitope containing peptide
PFQGAHRLTWAKIGPVGAVV

SEQ ID NO: 412 - protein, 20 aa, Artificial sequence
  A myelin protein unique epitope containing peptide
VGLSVMYASWKPTVNGTMVA

SEQ ID NO: 413 - protein, 20 aa, Artificial sequence
  A myelin protein unique epitope containing peptide
YSHSDLGKQPTKDSYTLTEE

SEQ ID NO: 414 - protein, 20 aa, Artificial sequence
  A myelin protein unique epitope containing peptide
YFNTWTTCDSIAFPSKTSAS

SEQ ID NO: 415 - protein, 20 aa, Artificial sequence
  A myelin protein unique epitope containing peptide
RMYGVLPWIAFPGKVCGSNL

SEQ ID NO: 416 - protein, 20 aa, Artificial sequence
  A myelin protein unique epitope containing peptide
AHSLERVCTCLGKWLGHPDK

SEQ ID NO: 417 - protein, 20 aa, Artificial sequence
  A myelin protein unique epitope containing peptide
YNWLHRRLAGQFLEELRNPF

SEQ ID NO: 418 - protein, 20 aa, Artificial sequence
  A myelin protein unique epitope containing peptide
LLQITVGLIFLCLQYRLRGK

SEQ ID NO: 419 - protein, 20 aa, Artificial sequence
  A myelin protein unique epitope containing peptide
PVLLLQITLGLVFLCLQYRL

SEQ ID NO: 420 - protein, 20 aa, Artificial sequence
  A myelin protein unique epitope containing peptide
ASLVATGLGFFGVALFCGCG

SEQ ID NO: 421 - protein, 20 aa, Artificial sequence
  A myelin protein unique epitope containing peptide
KKMPFLELHFLKQESFPTSE

SEQ ID NO: 422 - protein, 20 aa, Artificial sequence
  A myelin protein unique epitope containing peptide
SLARFVAPTFCPSAKQLSSK

SEQ ID NO: 423 - protein, 20 aa, Artificial sequence
  A myelin protein unique epitope containing peptide
NAFPGKVCCSNLLSICKTAE

SEQ ID NO: 424 - protein, 20 aa, Artificial sequence
  A myelin protein unique epitope containing peptide
RPTKDSYTLTEELAECCQGP

SEQ ID NO: 425 - protein, 20 aa, Artificial sequence
  A myelin protein unique epitope containing peptide
EIENLHRTFGQFLEELPSRI

SEQ ID NO: 426 - protein, 20 aa, Artificial sequence
  A myelin protein unique epitope containing peptide
ICGKGLSATFVGITYALTVV

SEQ ID NO: 427 - protein, 20 aa, Artificial sequence
  A myelin protein unique epitope containing peptide
MIAATYNFAILKLMGRGTKF

SEQ ID NO: 428 - protein, 20 aa, Artificial sequence
  A myelin protein unique epitope containing peptide
LLQVSSSYADPFYWVSPGVL

SEQ ID NO: 429 - protein, 20 aa, Artificial sequence
  A myelin protein unique epitope containing peptide
GGRDSRSGSPMARRHHHHHH

SEQ ID NO: 430 - protein, 20 aa, Artificial sequence
  A myelin protein unique epitope containing peptide
ERTIPITREEKPAVTAAPKK

SEQ ID NO: 431 - protein, 20 aa, Artificial sequence
  A myelin protein unique epitope containing peptide
REGGRRPETRIWLRRQSVRL

SEQ ID NO: 432 - protein, 20 aa, Artificial sequence
  A myelin protein unique epitope containing peptide
RTPPPSQGKREGGRRPETRI

SEQ ID NO: 433 - protein, 20 aa, Artificial sequence
  A myelin protein unique epitope containing peptide
RTPPPSQGKGAEGQRPGFGY

SEQ ID NO: 434 - protein, 20 aa, Artificial sequence
  A myelin protein unique epitope containing peptide
SLPQKSQRSQDENPVVHFFK

SEQ ID NO: 435 - protein, 20 aa, Artificial sequence
  A myelin protein unique epitope containing peptide
MQFRVIGPRHPIRALVGDEV

SEQ ID NO: 436 - protein, 20 aa, Artificial sequence
  A myelin protein unique epitope containing peptide
ASTMDHARHGSLPQKSHGRT

(19) United States
(12) Patent Application Publication
(10) Pub. No.: US 2010/0150868 A1
(43) Pub. Date: Jun. 17, 2010

(54) Methods of Individually Optimizing Treatment for an Inflammation Associated Disease

(75) Inventors: Anat Achiron, Tel-Aviv (IL); Michael Gurevich, Rechovot (IL)

Correspondence Address: MARTIN D. MOYNIHAN d/b/a PRTSI, INC., P.O. BOX 16446, ARLINGTON, VA, 22215, US

(73) Assignee: Tel HaShomer Medical Research Infrastructure and Services Ltd., Ramat-Gan (IL)

(21) Appl. No.: 11/886,772

(22) PCT Filed: Mar. 21, 2006
(86) PCT No.: PCT/IL2006/000357

§ 371 Date: Jan. 6, 2009

Related U.S. Application Data
(60) Provisional application No. 60/663,234, filed on Mar. 21, 2005.

(51) Int. Cl. A61K 38/21; C12Q 1/02; A61K 39/395; A61K 31/195; A61K 31/44; A61K 31/606; A61P 29/00; C40B 40/10

(52) U.S. Cl. 424/85.6; 435/29; 424/173.1; 514/561; 514/352; 514/166; 514/565; 506/18

(57) ABSTRACT

A method of individually optimizing a treatment for an inflammation associated disease is provided. The method comprising contacting each of identical white blood cell samples of a subject in need thereof with a different pharmaceutical agent of a plurality of pharmaceutical agents for the inflammation associated disease, so as to allow elicitation of an anti-inflammatory activity in the white blood cell samples; assaying the anti-inflammatory activity in the white blood cell samples; and identifying a pharmaceutical agent of the plurality of pharmaceutical agents eliciting a strongest anti-inflammatory activity, the pharmaceutical agent being the individually optimized treatment for the inflammation associated disease, wherein when the inflammation associated disease is multiple sclerosis the white blood cell samples are inflamed white blood cell samples. Methods of treating an inflammation associated disease are also described.

### FIELD AND BACKGROUND OF THE INVENTION

The present invention relates to methods for individually optimizing drug treatment for an inflammation associated disease and methods of treating same.

The inflammatory response serves the purpose of eliminating harmful agents from the body. There is a wide range of pathogenic insults that can initiate an inflammatory response including infection, allergens, autoimmune stimuli, immune response to transplanted tissue, noxious chemicals, and toxins, ischemia/reperfusion, hypoxia, mechanical and thermal trauma. Inflammation is typically a localized action which serves in expulsion, attenuation by dilution, and isolation of the damaging agent and injured tissue. The body's response becomes an agent of disease when it results in inappropriate injury to host tissues in the process of eliminating the targeted agent, or responding to a traumatic insult.

Multiple Sclerosis (MS) is the most common autoimmune disease involving the nervous system. The disease affects twice as many women as it does men. There are 350,000 persons affected with MS in the U.S. alone with more than 10,000 new cases reported each year. Worldwide, MS affects nearly 2.5 million individuals. There is a high economic burden associated with the disease. The total annual cost for all people with MS in the U.S. has been estimated to be more than $9 billion dollars. [Whetten-Goldstein, K., et al., Mult Scler 4, 419-425 (1998)].

Clinically, the disease can be broadly divided into a relapsing remitting (RR) form characterized by a series of exacerbations that result in varying degrees of disability from which the patient recovers, and a progressive form in which the patient does not experience exacerbations, but instead reports a gradual decline. A relapsing-remitting onset is observed in 85-90% of patients. The course of the disease in about 40% of relapsing-remitting patients ultimately changes to a progressive form.

The hallmark of the disease is a well-demarked area of myelin loss, known as a “demyelinated plaque”. Symptoms are believed to occur from axonal demyelination that inhibits or blocks conduction. Plaques may be found throughout the brain and spinal cord. Inflammatory cells are seen at the edges of the plaque and scattered throughout the white matter. Amelioration of symptoms has been attributed to partial remyelination and resolution of inflammation. Based on accumulating data from immunological studies of MS patients and a wealth of animal model data, autoimmune dysregulation has been viewed as the major contributor to tissue damage.

MS pathological studies indicate autoantibodies against a specific myelin protein may mediate target membrane damage in central nervous system demyelinating disease [Genain, C. P. et al., 1999, Nat Med 5, 170-175]. In addition, antibodies specific for myelin basic protein (MBP), proteolipid protein (PLP), myelin associated glycoprotein (MAG), transaldoase (TAL) and myelin oligodendrocyte glycoprotein (MOG) have been identified in the cerebrospinal fluid of patients with MS.

There is increasing evidence indicating MS is associated with autoimmune inflammation involving activation and aberrant trafficking of T cells and other inflammatory cells which produce an array of inflammatory molecules such as cytokines, chemokines, their receptors and molecules related to T cell adhesion, trafficking and apoptosis [Ahmed et al., 2002 Am J Pathol. November; 161(5):1577-86]. The production of these molecules not only characteristically reflects the in vivo activity of inflammatory cells but also has clinical relevance to disease activity in MS.

There are indications that the changes in some of these serum inflammatory molecules correlate with brain lesion activity as measured by magnetic resonance imaging (MRI) as well as clinical progression in MS [Adachi et al., Ann Neurol. 1990 November; 28(5):687-91].

Relapsing remitting multiple sclerosis (RRMS) patients are typically treated with immunomodulatory drugs. Examples of such include, glatiramer acetate, β-IFNs (including IFN-β-1α and IFN-β-1β) and intravenous immunoglobulins (IVIG). All these immunomodulatory drugs are known to reduce pro-inflammatory cytokine production. Specifically, Glatiramer acetate and β-IFNs affect antigen presentation and the cytokine milieu. Glatiramer acetate leads to the formation of specific Th2 cells with immunoregulatory properties whereas β-IFNs inhibit expansion of autoreactive T cells. Similarly, one of the mechanisms of action of IVIG is modulation of cytokine release [Ibanez C, et al., BioDrugs 2005; 19:59-65].

The clinical decision related to which immunomodulatory treatment will be initiated in a specific RRMS patient is currently arbitrary. For example, some patients respond to β-IFN but not glatiramer acetate, or vice versa. Moreover, immunomodulatory treatments are not effective in all patients and the individual response to each drug can vary. Thus, a biologic method that will help to choose the appropriate immunomodulatory treatment for each patient could prove cost effective and more importantly be of clinical value.

It has been difficult to evaluate in a timely fashion the treatment effect of both β-IFN and glatiramer acetate in MS patients because of the slowly progressive nature of the disease and because of the low sensitivity of current clinical measurements. For both β-IFN and glatiramer acetate, it often takes 3-9 months before clinical effects become measurable in patients that respond to the treatments (Jacobs et al., 1996, Ann Neurol 39,285). As a result, valuable time may be lost in analyzing whether a selected treatment is optimal for a particular individual.

Although advanced magnetic resonance imaging (MRI) technology represents a suitable research tool to assess the activity of the CNS pathology, its routine and frequent utility for treatment monitoring in MS is limited.

U.S. Pat. Appl. No. 20030092089 teaches diagnosing or monitoring multiple sclerosis by analyzing levels of auto-antibodies. U.S. Pat. Appl. No. 20030092089 does not mention or suggest using this assay for evaluating individual efficacy of drug treatments.

U.S. Pat. Appl. No. 20050064483 teaches a gene expression profiling assay for treatment evaluation of multiple sclerosis. Specifically, the assay comprises contacting a sample of peripheral blood mononuclear cells obtained from a MS patient with a drug and analyzing the cellular RNA to see whether particular genes are up-regulated. The genes which may be analyzed include cytokines. U.S. Pat. Appl. No. 20050064483 neither teaches selecting the clinical sample during a relapsed stage of the individual, nor teaches stimulating the sample to mimic such a relapse, suggesting that results obtained using this method cannot be reliably used to ascertain an optimal treatment for a patient during remission from the disease.

U.S. Pat. Appl. No. 20050064516 teaches a method of assessing the efficacy of a treatment for multiple sclerosis in a subject by analyzing multiple sclerosis markers. U.S. Pat. Appl. No. 20050064516 teaches administration of a particular drug to a patient followed by obtaining a biological sample from the patient and analyzing the set of markers. Since this method cannot analyze more than one treatment at time, it cannot be applied for selecting an optimal drug treatment for a particular individual. In addition, use of the patient as an in-vivo test-tube, may expose the patient to non-favorable treatments.

There is thus a widely recognized need for, and it would be highly advantageous to have, a simple and sensitive in vitro bioassay capable of selecting an optimal individual drug treatment for an inflammation associated disease.

### SUMMARY OF THE INVENTION

According to one aspect of the present invention there is provided a method of individually optimizing a treatment for an inflammation associated disease, the method comprising: (a) contacting each of identical white blood cell samples of a subject in need thereof with a different pharmaceutical agent of a plurality of pharmaceutical agents for the inflammation associated disease, so as to allow elicitation of an anti-inflammatory activity in the white blood cell samples; (b) assaying the anti-inflammatory activity in the white blood cell samples; and (c) identifying a pharmaceutical agent of the plurality of pharmaceutical agents eliciting a strongest anti-inflammatory activity, the pharmaceutical agent being the individually optimized treatment for the inflammation associated disease, wherein when the inflammation associated disease is multiple sclerosis the white blood cell samples are inflamed white blood cell samples.

According to another aspect of the present invention there is provided a method of treating an inflammation associated disease in a subject, the method comprising: (a) contacting each of identical white blood cell samples of the subject with a different pharmaceutical agent of a plurality of pharmaceutical agents for the inflammation associated disease, so as to allow elicitation of an anti-inflammatory activity in the white blood cell samples; (b) assaying the anti-inflammatory activity in the white blood cell samples; (c) identifying a pharmaceutical agent of the plurality of pharmaceutical agents eliciting a strongest anti-inflammatory activity, the pharmaceutical agent being the individually optimized treatment for the inflammation associated disease; and (d) administering the pharmaceutical agent eliciting the strongest anti-inflammatory activity to the subject, wherein when the inflammation associated disease is multiple sclerosis, the white blood cell samples are inflamed white blood cell samples, thereby treating an inflammation associated disease in the subject.

According to yet another aspect of the present invention there is provided a method of assessing the efficacy of a pharmaceutical agent for individually treating an inflammation associated disease, the method comprising: (a) contacting a white blood cell sample of a subject in need thereof with a pharmaceutical agent for the inflammation associated disease, so as to allow elicitation of an anti-inflammatory activity in the white blood cell sample; and (b) assaying the anti-inflammatory activity in the white blood cell samples, wherein an anti-inflammatory activity above a predetermined threshold is indicative of therapeutic efficacy of the pharmaceutical agent, wherein when the inflammation associated disease is multiple sclerosis, the white blood cell samples are inflamed white blood cell samples, thereby assessing the efficacy of a pharmaceutical agent for individually treating an inflammation associated disease.

According to further features in preferred embodiments of the invention described below, the inflammation associated disease is an autoimmune disease.

According to still further features in the described preferred embodiments, the white blood cell samples are inflamed white blood cell samples.

According to still further features in the described preferred embodiments, the method further comprises contacting the white blood cell samples with at least one autoantigen of the autoimmune disease so as to obtain the inflamed blood cell samples prior to step (a).

According to still further features in the described preferred embodiments, least one autoantigen is selected by: (a) contacting a plurality of white blood cell samples of the subject with a plurality of peptides; and (b) selecting at least one peptide of the plurality of peptides that elicits an immune activity above a predetermined threshold, the peptide being the autoantigen that activates white blood cells of the individual subject with the autoimmune disease.

According to still further features in the described preferred embodiments, each of the plurality of peptides comprise a specific epitope for the autoimmune disease.

According to still further features in the described preferred embodiments, the subject is in remission from the autoimmune disease.

According to still further features in the described preferred embodiments, the subject is free of anti-inflammatory treatments for at least 30 days prior to the treating.

According to still further features in the described preferred embodiments, the white blood cell samples comprise peripheral blood mononuclear cells.

According to still further features in the described preferred embodiments, the autoimmune disease is selected from the group consisting of rheumatoid arthritis, rheumatoid spondylitis, osteroarthritis, gouty arthritis, arthritic conditions, inflamed joints, eczema, inflammatory skin conditions, inflammatory eye conditions, conjunctivitis, pyresis, tissue necrosis resulting from inflammation, tissue rejection following transplant surgery, Crohn's disease and ulcerative colitis, airway inflammation, asthma, bronchitis, systemic lupus erythematosis, multiple sclerosis, myasthenia gravis, progressive systemic sclerosis, atopic dermatitis, hyperimmunoglobin E, hepatitis B antigen negative chronic active hepatitis, Hashimoto's thyroiditis, familial Mediterranean fever, Grave's disease, autoimmune haemolytic anemia, primary biliary cirrhosis, inflammatory bowel disease, viral infections, HIV infections and AIDS.

According to still further features in the described preferred embodiments, auto-immune disease is multiple sclerosis.

According to still further features in the described preferred embodiments, autoimmune disease is Crohns disease.

According to still further features in the described preferred embodiments, the pharmaceutical agent is selected from the group consisting of interferon-β-1-α, interferon-β-1-β, an immunoglobulin and glatiramer acetate. According to still further features in the described preferred embodiments, pharmaceutical agent is selected from the group consisting of a 5A5A compound, sulfasalazine, mesalamine and olsalazine.

According to still further features in the described preferred embodiments, assaying anti-inflammatory activity comprises: (i) assaying an activity and/or expression of an anti inflammatory cytokine; (ii) assaying an activity and/or expression of a pro-inflammatory cytokine; and/or (iii) assaying a ratio of (i) to (ii).

According to still further features in the described preferred embodiments, the pro-inflammatory cytokine is selected from the group consisting of interleukin 1 (IL1), interleukin 2 (IL2), interleukin 6 (IL6), interleukin 7 (IL7), interleukin 8 (IL8), interleukin 9 (IL9), interleukin 12 (IL12), interleukin 15 (IL15), interferon gamma (IFNγ) and tumor necrosis factor (TNF-α).

According to still further features in the described preferred embodiments, the pro-inflammatory cytokine is TNF-α.

According to still further features in the described preferred embodiments, the anti-inflammatory cytokine is selected from the group consisting of transforming growth factor beta (TGFβ), interferon alpha (IFNα), interferon beta (IFNβ), interleukin 4 (IL4) and interleukin 10 (IL 10).

According to still further features in the described preferred embodiments, assaying the anti-inflammatory activity is effected at the mRNA level.

According to still further features in the described preferred embodiments, assaying the anti-inflammatory activity is effected at the protein level.

According to still further features in the described preferred embodiments, an assay at the mRNA level is selected from the group consisting of an RT-PCR assay, a northern assay, an oligonucleotide microarray assay

According to still further features in the described preferred embodiments, an assay at the protein level is selected from the group consisting of an immunoassay, a flow cytometry assay a receptor assay and an activity assay.

According to still further features in the described preferred embodiments, the at least one auto-antigen is selected from the proteins consisting of Myelin-associated Glycoprotein (MAG), Myelin-oligodendrocyte Glycoprotein (MOG), Myelin Basic Protein (MBP) and Proteolipid Protein (PLP).

According to still further features in the described preferred embodiments, the at least one auto-antigen does not comprise more than 20 amino acids peptides.

According to still further features in the described preferred embodiments, the amino acid peptides are selected from the group as set forth in Table 2.

According to still further features in the described preferred embodiments, the at least one auto-antigen comprises an active epitope.

According to still further features in the described preferred embodiments, an assayable amount of the interferon-β-1-α is selected from the range of 20-50 units per milliliter.

According to still further features in the described preferred embodiments, an assayable amount of the interferon-β-1-β is selected from the range of 10-30 units per milliliter.

According to still further features in the described preferred embodiments, an assayable amount of the immunoglobulin is selected from the range of 1.5-4 mg/ml.

According to still further features in the described preferred embodiments, an assayable amount of the glatiramer acetate is selected from the range of 5-15 mg/ml.

According to still another aspect of the present invention there is provided a kit to optimize treatment against an inflammatory autoimmune disease, the kit comprising a packaging material which comprises at least one autoantigen peptide for the autoimmune disease.

According to still further features in the described preferred embodiments, the kit further comprises components for assaying an anti-inflammatory activity.

According to still further features in the described preferred embodiments, the inflammatory autoimmune disease is multiple sclerosis.

According to an additional aspect of the present invention there is provided an array comprising a set of epitopes selected from the group of 20 amino acid peptides as set forth in Table 2.

According to yet an additional aspect of the present invention there is provided a method of selecting an auto-antigen that activates white blood cells of a subject with an autoimmune disease, the method comprising: (a) contacting a plurality of white blood cell samples of the subject with a plurality of peptides each comprising a specific epitope for the autoimmune disease; and (b) selecting at least one peptide of the plurality of peptides that elicits an immune activity above a predetermined threshold, the peptide being the autoantigen that activates white blood cells of the individual subject with the autoimmune disease.

According to still further features in the described preferred embodiments, the plurality of peptides are attached to a solid support in an addressable manner.

According to still further features in the described preferred embodiments, the plurality of peptides are set forth in Table 2.

The present invention successfully addresses the shortcomings of the presently known configurations by providing a method of selecting an individually optimized treatment for an inflammation associated disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-D are bar graphs illustrating the number of patients whose peripheral blood mononuclear cells showed a decrease in TNF-α levels following stimulation with immunomodulatory drugs and Myelin-oligodendrocyte Glycoprotein (MOG). FIG. 1A is a bar graph illustrating the response of a typical patient which showed a positive response to one particular drug (in this case drug 2). Altogether, 21 patients (38% of total patients tested) responded in a similar way. FIG. 1B is a bar graph illustrating the response of a typical patient which showed a positive response to two particular drugs (in this case, drugs 2 and 3). Altogether 14 patients (26% of total patients) responded in a similar way. FIG. 1C is a bar graph illustrating the response of a typical patient which showed a positive response to all the drugs tested. Altogether 11 patients (20% of total patients) showed a similar response. FIG. 1D is a bar graph illustrating the response of a typical patient which showed a negative response to the tested drugs. Altogether, 8 patients (15% of total patients) showed a negative response to the drugs. Treatment 1 in FIG. 1A-D refers to MOG alone.

FIG. 2 is a bar graph comparing the acute (16 weeks) relapse rate in patients matched with the immunomodulatory drug matching method of the present invention to the acute relapse rate in non-matched patients.

FIG. 3 is a bar graph comparing the long-term (1 year) relapse rate in patients matched with the immunomodulatory drug matching method of the present invention to the relapse rate in non-matched patients.

FIG. 4 is a bar graph comparing time to next relapse in patients matched with the immunomodulatory drug matching method of the present invention to the time to next relapse in non-matched patients during 1 year follow up.

FIG. 5 is a diagrammatic representation of the algorithm used to select a set of 20 amino acid peptides that incorporates all existing 12 amino acid peptide variants within myelin proteins.

### DESCRIPTION OF THE PREFERRED EMBODIMENTS

The present invention is of a method of individually optimizing treatment for an inflammation associated disease. Specifically, the present invention can be used to select the most favorable pharmaceutical agents for the treatment of autoimmune diseases such as multiple sclerosis.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Multiple sclerosis is an inflammatory autoimmune disorder involving activation and aberrant trafficking of T cells and other inflammatory cells which produce an array of inflammatory molecules such as cytokines, chemokines, their receptors and molecules related to T cell adhesion, trafficking and apoptosis.

Relapsing remitting multiple sclerosis (RRMS) patients are typically treated with immunomodulatory drugs. The clinical decision related to which immunomodulatory treatment will be initiated in a specific RRMS patient is currently arbitrary. Immunomodulatory treatments are not effective in all patients and the individual response to each drug can vary. Accordingly, a biologic method that will help to choose the appropriate immunomodulatory treatment for each patient could prove cost effective and more importantly clinically valuable.

U.S. Pat. Appl. No. 20030092089 teaches diagnosing or monitoring multiple sclerosis by analyzing levels of auto-antibodies. U.S. Pat. Appl. No. 20030092089 does not mention or suggest using this assay for evaluating the efficacy of drug treatments for a particular individual.

U.S. Pat. Appl. No. 20050064516 teaches a method of assessing the efficacy of a treatment for multiple sclerosis in a subject by analyzing multiple sclerosis markers. U.S. Pat. Appl. No. 20050064516 teaches administration of a particular drug to a patient followed by obtaining a biological sample from the patient and analyzing the set of markers. This method cannot analyze more than one treatment at time and accordingly cannot be applied for selecting an optimal drug treatment out of a number of treatments for a particular individual. In addition, use of the patient as an in-vivo test-tube, may expose the patient to non-favorable treatments.

While reducing the present invention to practice, the present inventors have uncovered a novel approach for individually optimizing treatment for an inflammation associated disease from a plurality of pharmaceutical agents commonly used for treating the disease.

As is illustrated in the Examples section which follows, the present inventors devised an ex-vivo assay wherein a selection of immunomodulatory drugs were added to inflamed white blood cell samples of a multiple sclerosis patient and the reduction of TNF-α in each sample was measured. The drug that decreased TNF-α to the greatest extent was selected as being the drug of choice for a particular individual (see Example 2). By performing short-term and long-term clinical studies, the present inventors showed that an immunomodulatory drug that had been selected according to the above described ex-vivo assay was the most preferable drug for the treatment of that multiple sclerosis patient (Example 2).

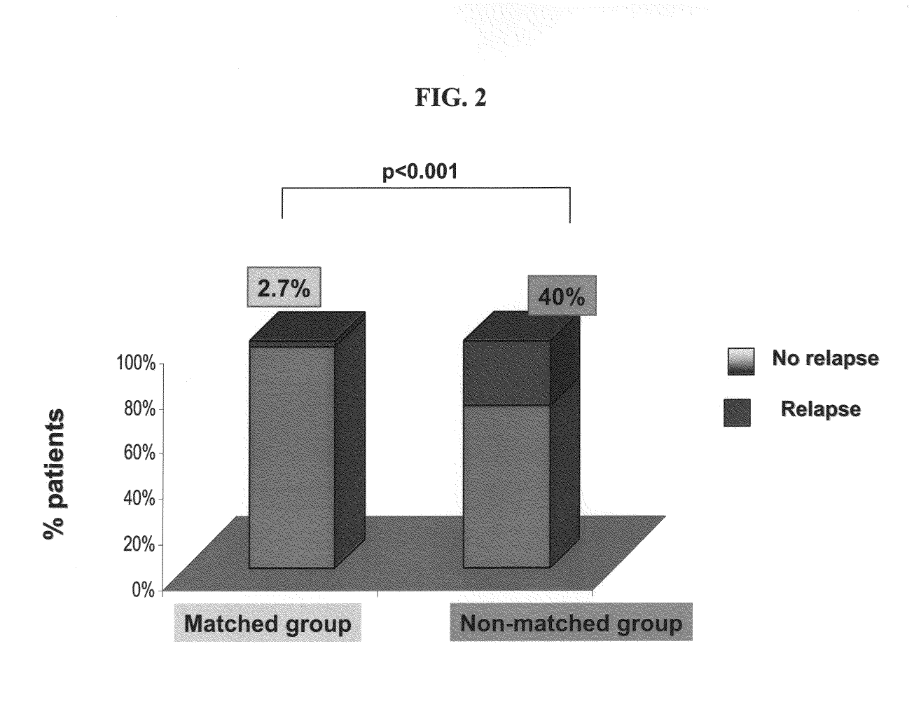

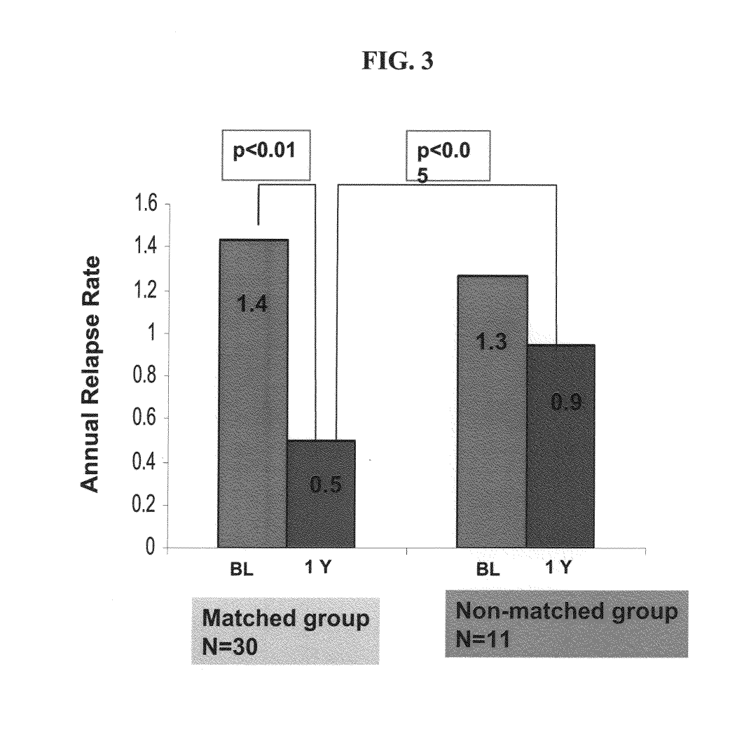

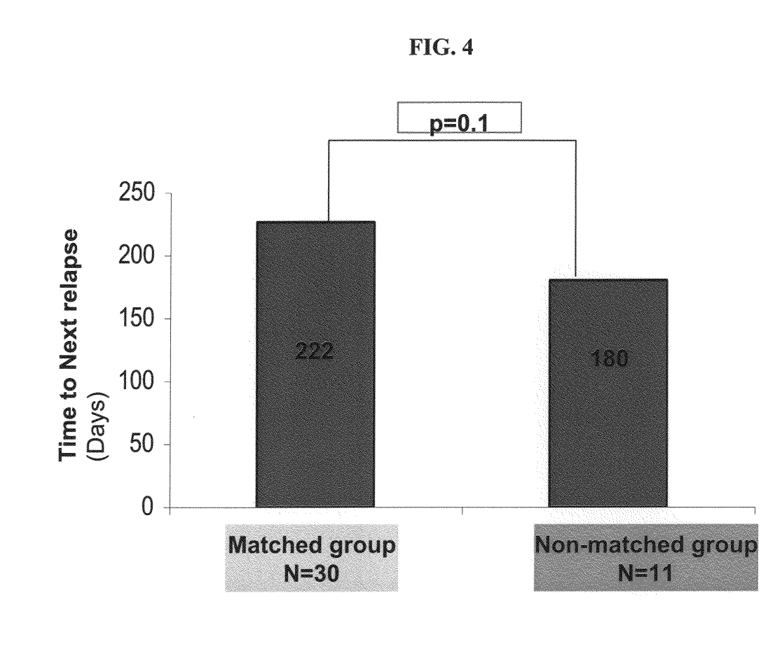

Thus, in a short-term clinical trial, the inventors have shown that only 2.7% of patients had an acute relapse whilst being treated with a drug selected according to the assay of the present invention, while 40% of patients developed a relapse whilst being treated with a drug that wasn't selected according to the assay of the present invention (FIG. 2). Furthermore, in a long-term clinical trial, the relapse rate significantly decreased in the matched group, while in the non-matched group no significant change in relapse rate occurred, suggesting usefulness of the invention. (FIG. 3). In addition, the time between relapses was reduced from 222±29 days in the matched group and 180±21 days in the non-matched group (FIG. 4).

U.S. Pat. Appl. No. 20050064483 teaches a gene expression profiling assay for treatment evaluation of multiple sclerosis. Specifically, the assay comprises contacting a sample of peripheral blood mononuclear cells obtained from a MS patient with a drug and analyzing the cellular RNA to see whether particular genes are up-regulated. The genes which may be analyzed include cytokines.

In sharp contrast to the present invention, U.S. Pat. Appl. No. 20050064483 does not teach using inflamed white blood cells, neither by selecting the clinical sample during a relapsed stage of the individual, nor by stimulating the sample exogenously to mimic such a relapse.

Thus, according to one aspect of the present invention, there is provided a method of individually optimizing treatment for an inflammation associated disease. The method comprises contacting each of identical white blood cell samples of a subject in need thereof with a different pharmaceutical agent of a plurality of pharmaceutical agents being commonly used for treating the inflammation associated disease, so as to allow elicitation of an anti-inflammatory activity in the white blood cell samples; assaying the anti-inflammatory activity in the white blood cell samples; and identifying a pharmaceutical agent of the plurality of pharmaceutical agents eliciting a strongest anti-inflammatory activity, the pharmaceutical agent being the individually optimized treatment for the inflammation associated disease, wherein when the inflammation associated disease is multiple sclerosis the white blood cell samples are inflamed white blood cell samples.

As used herein, the phrase, “inflammation associated disease” refers to any disease or disorder which includes a component of inflammation, which is imperative to disease onset or progression. The inflammation associated disease may be a chronic or a relapsing remitting disease. According to an embodiment of this aspect of the present invention, the inflammation associated disease is an autoimmune disease. Herein, the phrase “autoimmune disease” refers to a disease resulting from a disordered immune reaction (e.g., antibody production) generated against components of one's own body (i.e. autoantigens). The immune system of the subject then activates an inflammatory cascade aimed at cells and tissues presenting those specific self antigens. The destruction of the antigen, tissue, cell type, or organ attacked by the individual's own immune system gives rise to the symptoms of the disease. According to a preferred embodiment of the present invention, the autoimmune disease is multiple sclerosis. Other examples of autoimmune and other inflammation associated diseases are detailed herein below.

The term “autoantigen” as used herein refers to a molecule derived from a subject, typically a polypeptide molecule comprising one or more epitopes, capable of eliciting an immune response in that subject. This is in contrast with antigens which are foreign, or exogenous, which are not normally part of the subject's milieu. Each autoimmune disease is characterized by an immune response directed at an autoantigen. Thus, for example, autoantigens for multiple sclerosis include, but are not limited to Myelin-associated Glycoprotein (MAG), Myelin-oligodendrocyte Glycoprotein (MOG), Myelin Basic Protein (MBP) or Proteolipid Protein (PLP), or parts thereof.

The phrase “subject in need thereof” as used herein, typically refers to a human subject. Typically, the subject has been diagnosed with the inflammation associated disease. The subject may or may not have received treatment for the inflammation associated disease. In order to ensure the blood sample of the subject is not tainted by any in-vivo administered pharmaceutical agents which might prejudice the results of the assay of the present invention, the subject is preferably free of anti-inflammatory treatments (e.g. immunomodulatory treatments and/or steroids) for at least 10 days, more preferably 20 days and even more preferably 30 days prior to the assay. Examples of patients who may be free of anti-inflammatory treatments include patients who are in remission from an auto-immune disease, untreated patients who have never received previous treatment or switching patients who have stopped previous uneffective treatments.

Thus, as mentioned, the method of this aspect of the present invention is affected by contacting identical white blood cells with a plurality of pharmaceutical agents.

As used herein, the phrase, “white blood cells” refers to bone marrow derived blood cells which are part of the immune system responsible for both cellular (e.g., T cells and macrophages) and humoral (B-cells producing antibodies) immune response. Examples of white blood cells include macrophages, B- and T-lymphocytes, monocytes, neutrophiles, eosinophiles, and basophiles.

Preferably, the white blood cell samples include peripheral blood mononuclear cells. The phrase, “peripheral blood mononuclear cells” (PBMCs) as used herein, refers to a mixture of monocytes and lymphocytes. Several methods for isolating white blood cells are known in the art. For example, PBMCs can be isolated from whole blood samples using density gradient centrifugation procedures. Typically, anticoagulated whole blood is layered over the separating medium. At the end of the centrifugation step, the following layers are visually observed from top to bottom: plasma/platelets, PBMCs, separating medium and erythrocytes/granulocytes. The PBMC layer is then removed and washed to remove contaminants (e.g., red blood cells) prior to optional cell typing and cell viability assays. Alternatively, PBMCs may be isolated using a ficol-hypaque gradient as described in Example 2 of the Examples section hereinbelow.

The white blood cells of the present invention may be in suspension or cultured. An exemplary culturing medium for PBMCs includes complete RPMI 1640 containing 10% CCS and gentamicin/penicillin/streptamicin (Gibco, Grand Island, N.Y.). Typically, PBMCs are seeded in 96 well plates at a density of 2.5×10⁵ cells/well in 200 μl of the above described medium.

The white blood cell samples of the present invention, may be homogeneous or heterogeneous cell samples (two or more cell types) and may comprise additional cells (red blood cells) as long as their in vitro functionality is retained (e.g., ability to secrete cytokines).

Identical white blood cell samples of this aspect of the present invention are preferably aliquots of a single or pooled white blood cell samples.

As mentioned herein above, the method of this aspect of the present invention may be used to individually optimize treatment for an auto-immune disease. Preferably, the white blood cell samples from an auto-immune subject are inflamed prior to commencement of the assay (i.e., contacting with the pharmaceutical agents as mentioned hereinabove).

As used herein, the term “inflamed” refers to white blood cells that have been activated by an autoantigen so that following activation the cells typically secrete inflammatory cytokines.

White blood cells may be inflamed in vivo i.e. inflamed by an autoantigen as a natural course of the auto-immune disease (e.g., from a relapsed subject). Alternatively, or additionally non-inflamed white blood cells may be withdrawn from the subject and may be inflamed by contacting the samples with at least one autoantigen associated with the relevant auto-immune disease. Ex vivo stimulation of white blood cells with an autoantigen is typically effected for a time between 24 hours to 72 hours. The autoantigen is typically added at a concentration of 10-50 μ/ml.

Thus, a patient who is in remission from the autoimmune diseases, whose white blood cells may not be inflamed in vivo, may still ascertain the optimal treatment for his/her autoimmune disease by ex-vivo white blood cell inflammation prior to contacting with a pharmaceutical agent.

Preferably, the autoantigen used to inflame the white blood cells comprises at least one active epitope. Methods of determining an active epitope are further described hereinbelow.

Once identical blood samples are obtained they are each contacted with a pharmaceutical agent from a plurality of pharmaceutical agents that are typically used to treat a particular inflammation associated disease. Typically, the pharmaceutical agents share a common mechanism of action or at least a partly common mechanism of action. Pharmaceutical compositions of the present invention preferably elicit an anti inflammatory activity.

As used herein the phrase “anti-inflammatory activity” refers to an activity that reduces or prevents any component of inflammation.

Thus, for example one group of pharmaceutical agents that may be assayed for individually optimizing treatment of multiple sclerosis are those pharmaceutics that elicit an increase in the quantity of tumor necrosis factor (TNF-α). Examples of such pharmaceutical agents include, but are not limited to interferon-β-1-α, interferon-β-1-β, an immunoglobulin and glatiramer acetate. Exemplary pharmaceutical agents that may be assayed for individually optimizing treatment of Crohn's disease, are those pharmaceutics that elicit an increase in TNF-α, interleukin-12, and interferon-γ. Examples of such pharmaceutical agents include, but are not limited to 5A5A compound, sulfasalazine, mesalamine and olsalazine.

Establishment of appropriate concentrations of the pharmaceutical agents can be effected by a comparison of the in-vivo treatment dose for each drug through equations with the in vitro culture environment. The in-vitro concentration for each drug may be calculated according to the serum levels acquired after in-vivo injections or according to functional equivalence tests. Alternatively or additionally, the appropriate concentrations may be established by in-vitro calibration assays.

Accordingly, as described in Example 1, an assayable amount of interferon-β-1-α may be selected from the range of 20-50 units per milliliter. An assayable amount of interferon-β-1-β may be selected from the range of 10-30 units per milliliter. An amount of immunoglobulin may be selected from the range of 1.5-4 mg/ml. An amount of glatiramer acetate may be selected from the range of 5-15 mg/ml.

Anti inflammatory activity may be analyzed by assaying an activity and/or expression of an anti inflammatory cytokine; assaying an activity and/or expression of a pro-inflammatory cytokine; and/or assaying a ratio of the above.

Specific examples of anti-inflammatory activities that may be assayed according to this aspect of the present invention include, but are not limited to a decrease in arachidonic acid derivatives (e.g. prostaglandins and leukotrienes) a decrease in a proinflammatory cytokine, an decrease in a proinflammatory cytokine receptor, an increase in an anti-inflammatory cytokine, an increase in an anti-inflammatory cytokine receptor and a decrease in other white blood cell derived inflammatory mediators such as platelet activating factor, histamine, and bradykinin. In addition, an increase in adhesion molecules such as integrins may be assayed according to this aspect of the present invention. The present invention also envisages assaying more than one anti-inflammatory activity. In addition, a ratio between two anti-inflammatory activities can also be assayed (e.g. the decrease in a proinflammatory cytokine: increase in anti-inflammatory cytokine).

Examples of proinflammatory cytokines that may be assayed according to this aspect of the present invention include, but are not limited to interleukin 1 (IL1), interleukin 2 (IL2), interleukin 6 (IL6), interleukin 7 (IL7), interleukin 8 (IL8), interleukin 9 (IL9), interleukin 12 (IL12), interleukin 15 (IL15), interferon gamma (IFNγ) and tumor necrosis factor (TNF-α).

Examples of anti-inflammatory cytokines that may be assayed according to this aspect of the present invention include, but are not limited to transforming growth factor beta (TGFβ), interferon alpha (IFNα), interferon beta (IFNβ), interleukin 4 (IL4) and interleukin 10 (IL 10).

According to this aspect of the present invention, the anti-inflammatory activity of the pharmaceutical agents may be assayed at the mRNA level or the protein level. Examples of assays used to measure the quantity of mRNA are described hereinbelow.

Northern Blot analysis: This method involves the detection of a particular RNA in a mixture of RNAs. RNA may be extracted from white blood cells using methods known in the art. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from white blood cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls. The RT-PCR technique has been used successfully for the detection of cytokines. See e.g. O'Garra A and Vieira P, Current Opinion in Immunology, 1992, 4: 211-5.

An adaptation of RT-PCR is real-time PCR when the end product is measured in real-time. Real-time PCR has also been used for the detection of cytokines. See e.g. Giulietty A et al, Methods 2001 December; 25(4):386-401.

RNA in situ hybridization stain: In this method DNA or RNA probes are attached to the RNA molecules present in the white blood cells. Generally, the white blood cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using radio-labeled probes or to a colorimetric reaction which reveals signals generated using enzyme-linked labeled probes.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction may be performed on fixed white blood cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

Oligonucleotide microarray—In this method oligonucleotide probes capable of specifically hybridizing with the polynucleotides of the present invention are attached to a solid surface (e.g., a glass wafer). Each oligonucleotide probe is of approximately 20-25 nucleic acids in length. To detect the expression pattern of the polynucleotides of the present invention in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, Calif.) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Gene Chip Scanner, and the Microarray Suite software subtracts the non-specific signal resulting from the mismatch probe from the signal resulting from the perfect match probe. Use of microarrays to detect changes in cytokine concentration are described in U.S. Pat. Appl. No. 20050064483.

Examples of assays used to measure the quantity and activity of proteins are described hereinbelow.

Enzyme linked immunosorbent assay (ELISA): This method involves fixation of a sample (e.g., fixed white blood cells) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Enzyme linked immunospot assay (ELISPOT): The ELISPOT is an immunological assay based on ELISA and may be used to measure T cell activation. ELISPOTs rely on the principle that T cells secrete cytokines following activation. In this assay a given number of white blood cells (e.g. peripheral blood cells) are contacted with antigen (typically in a microtiter plate). The T cells settle to the bottom of the plate and, if they are specific for the given antigen, they will become activated. Because the plates are pre-coated with antibodies to the cytokine of interest, cytokines secreted by activated T cells will be “captured” locally. Typically, CD4 responses are measured by interleukin-4 capture, while CD8 responses are measured by IFN-γ capture. Use of ELIPSOT for measuring cytokines is known in the art. See e.g. Meierhoff, G, Diabetes Metab Res Rev. 2002, 18(5):367-80.

Western blot: A Western blot involves separation of proteins in an extract by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (e.g. the cytokine) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample. Use of RIA for the detection of cytokines is known in the art. See e.g. Meager A. In: Balkwill FR (edt.) Cytokines, a practical approach, IRL Press, Oxford, 1991, 299-307.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously. Flow cytometry has been successfully used for cytokine detection. See e.g. Pala P et al Journal of Immunological Methods, 2000, 243(1-2): 107-24;

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the cells containing an active enzyme and the enzyme catalyzes a reaction in which the substrate is decomposed to produce a chromogenic product visible by a light or a fluorescent microscope.

Receptor activation assay: In these methods the activity of a particular protein ligand is measured in a protein mixture extracted from the white blood cells by measuring ligand-induced receptor tyrosine kinase activation in terms of receptor phosphorylation. The assay, termed a ‘kinase receptor activation’ or KIRA, utilizes two separate microtiter plates, one for ligand stimulation of intact cells, and the other for receptor capture and phosphotyrosine ELISA. The assay makes use of either endogenously expressed receptors or stably transfected receptors with a polypeptide flag. This method was successfully used for the quantitation of cytokines [Sadick M D et al., Journal of Pharmacological and Biomedical Analysis 1999, 19(6): 883-9].

Another receptor assay which have been used for measuring cytokine activity is the radioreceptor assay (RRA). See e.g. Perret G and Simon P, Journal of Pharmacology 15: 265-286 (1984).

As mentioned above, the white blood cell samples from an auto-immune subject are preferably inflamed prior to commencement of the assay by contacting with an autoantigen.

The autoantigens used herein can be obtained and/or produced using a variety of methods known to those skilled in the art. In particular, the autoantigens can be isolated directly from native sources, using standard purification techniques. Alternatively, the autoantigens can be recombinantly produced using expression systems well known in the art and purified using known techniques. According to a preferred embodiment of the present invention, the autoantigens, such as the 20 amino acid peptides described hereinbelow are synthesized via chemical polymer syntheses such as solid phase peptide synthesis. Such methods are known to those skilled in the art. See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides: Analysis, Synthesis, Biology, supra, Vol. 1, for classical solution synthesis.

Autoimmune polypeptides may be packed in a kit for optimizing treatment against an inflammation autoimmune disease. The kit of the present invention may, if desired, be presented in a pack which may contain one or more units of the kit of the present invention. The pack may be accompanied by instructions for using the kit. The pack may also be accommodated by a notice associated with the container in a form prescribed by a governmental agency regulating the manufacture, use or sale of laboratory supplements, which notice is reflective of approval by the agency of the form of the compositions.

According to one aspect, the kit comprises at least one autoantigen peptide for an inflammatory autoimmune disease.

Additionally, the kit may comprise additional components for assaying anti-inflammatory activity preferably in a separate container. Examples of such components are described herein below.

According to a preferred embodiment of this aspect of the present invention, the autoantigens comprise at least one active epitope.

As used herein, the phrase “active epitope” refers generally to those features of an antigen which are capable of inducing a T cell response. A subject with an autoimmune disease typically displays an immune response to an individual repertoire of active epitopes. Furthermore, epitopes which are active at a particular stage of an autoimmune disease may become non-active during the course of that disease and vica versa. The active epitope on a particular autoantigen may spread to different epitopes on the same protein, i.e. “intramolecular epitope spreading”, or to other epitopes on other autoantigens, termed “intermolecular epitope spreading”. Typically, T cell active epitopes comprise linear peptide determinants that assume extended conformations within the peptide-binding cleft of MHC molecules, (Unanue et al. (1987) Science 236:551-557). Accordingly, an active epitope is generally a peptide having at least about 3-5 amino acid residues, and preferably at least 5-12 amino acid residues. Preferably such peptides are no more than 20 amino acids long.

Thus, according to one embodiment of this aspect of the present invention, the autoantigen is individually selected for a subject. An exemplary method of individually selecting an autoantigen comprises incubating white blood cell samples of the subject with a plurality of peptides and selecting the peptide or peptides that elicit the strongest immune activity. Preferably each of the plurality of peptides comprise a specific epitope for the autoimmune disease.

The plurality of peptides may comprise epitopes from one or preferably more than one protein known to be an autoantigen for the autoimmune disease. As mentioned above, exemplary autoantigens for multiple sclerosis include MAG and MOG derived peptides. Exemplary autoantigens for Crohns disease are known in the art—see e.g. Beiβbarth T et al., Bioinformatics, 2005, Vol. 21 Suppl, i29-i37. Other exemplary autoantigens that may be used according to this aspect of the present invention include, but are not limited to insulin, glutamic acid decarboxylase (64K), PM-1 and carboxypeptidase for diabetes; rh factor in erythroblastosis fetalis; acetylcholine receptors in myasthenia gravis; thyroid receptors in Graves' Disease; basement membrane proteins in Good Pasture's syndrome; and thyroid proteins in thyroiditis. Preferably, the plurality of peptides comprises as many possible epitopes from as many candidate autoantigen proteins for the autoimmune disease.

The plurality of peptides may be selected based on known methods for identifying hypothetical epitopes for a particular protein. An example of such a method implements an algorithm to generate a set of unique short peptide sequences that incorporate all possible epitopes within a group of proteins [Beiβbarth T et al., Bioinformatics, 2005, Vol. 21 Suppl, i29-i37]. This method was adapted by the present inventors to generate such short peptide sequences for all myelin proteins (MBP, PLP, MOG, MAG, CNPase, crystallin, S100beta). The sequences are set forth in Table 2 of Example 3, hereinbelow. Thus, according to a particular embodiment of this aspect of the present invention, the plurality of peptides is set forth in Table 2.

Alternatively, the plurality of peptides may be selected using other algorithms besides that described herein above in order to predict T cell epitopes (Bian et al. 2003, Methods, 29, 299-309). Yet alternatively, the plurality of peptides may comprise overlapping peptides (e.g. 15-20 amino acid peptides overlapping by 10-12 amino acids) spanning a protein of interest [Cease et al, 1987 Proc. Natl Acad. Sci. USA, 84, 4249-4253]. Still alternatively, the plurality of peptides may be selected from a combinatorial peptide library [Sospedra et al 2003, Methods, 29, 236-247] or from a plurality of peptides eluted from specific multi histocompatability complexes following incubation of an antigen presenting cell with an antigen [Lemel and Stevanovic, 2003, Methods, 29, 248-259].

As mentioned above the peptides or peptide that elicits an immune activity is selected as the autoantigen for inflaming a subject's blood sample according to the method of the present invention.

As used herein, the phrase “immune activity” refers to a T cell activity (e.g. secretion of inflammatory cytokines) and/or a B cell activity (e.g. presence of antibody) since it has been found that the specificity of an autoreactive T cell response can correlate with that of the autoreactive B cell response see e.g. U.S. Pat. App. No. 20030003516. Accordingly, in several human autoimmune diseases such as MS, the autoimmune T and B cell responses recognize substantially the same immunodominant epitopes.

The ability of a particular peptide to elicit an immune activity comprising an autoreactive T cell response in the white blood cell sample of a patient may be determined by a number of well-known assays, e.g. the ELISPOT T assay as described hereinabove. An autoreactive B cell response may by determined by assaying for the presence of antibodies in the subjects sample white blood cells. Regardless of the specific assay used to measure the immune activity, the peptide that elicits an immune activity above a predetermined threshold is selected as being immunogenic. The predetermined threshold may be determined by using known negative controls (e.g. an active epitope of an autoantigen of a non-related auto-immune disease). Preferably, the negative control comprises a substantially similar number of amino acids to the candidate autoantigen polypeptides being analyzed. A peptide that elicits at least a 10%, more preferably 30%, more preferably 50%, more preferably 70%, more preferably 100% or higher immunogenic response than that elicited by the negative control may be selected as being immunogenic.

The candidate autoantigen polypeptides may be added individually to each blood cell sample. Alternatively, the candidate autoantigen polypeptides may be immobilized on a solid support i.e. an array (such as a chip or a 96 well plate) and the white blood cells may be added as a suspension. Methods of immobilizing peptides on solid substrates are well known in the art. Such an array may comprise the peptides of Table 2.

As used herein, the term “array” refers to a plurality of addressable epitopes. The epitopes may be spacially addressable, such as in arrays contained within microtiter plates or printed on planar surfaces where each epitope is present at distinct X and Y coordinates. Methods for the manufacture and use of spatial arrays of polypeptides are known in the art. See e.g. Joos et al. (2000) Electrophoresis 21(13):2641-50; Roda et al. (2000) Biotechniques 28(3):492-6.

An alternative to this type of spatial coding array is the use of molecular “tags,” where the target epitopes are attached to a detectable label, or tag, which provides coded information about the sequence of the epitope. In a particular embodiment, a set of epitopes may be synthesized or attached to a set of coded beads, where each bead is linked to a distinct epitope, and where the beads are themselves coded in a manner that allows identification of the attached epitope. The use of a multiplexed microsphere set for analysis of clinical samples by flow cytometry is described in International Patent application no. 97/14028; and Fulton et al. (1997) Clinical Chemistry 43:1749-1756). It is also possible to use other addressable particles or tags (reviewed in Robinson et al. (2002) Arthritis Rheumatism 46:885-93).

As mentioned above, the array of the present invention may be used to determine autoantigen specificity for multiple sclerosis. Additionally, the array may be used to determine epitope spreading during the course of the disease, thereby acting as an aid in staging this autoimmune disease. In addition, an identified active epitope may be utilized to develop and select antigen or epitope specific therapies including: (1) oral administration of specific-antigens, termed ‘oral tolerance’ (Annu Rev Immunol. 12:809-37); (2) administration of native peptides (Science 258:1491-4; J Neurol Sci. 152:31-8); (3) administration of altered peptide ligands (Nature 379:343-5); (4) administration of whole proteins (Science 263:1139); administration of fusion-proteins or peptides; administration of other molecules, such as DNA or allergens including pollen, dust mites, cat salivary antigen (J. Rheumatology 28:257-65); administration of polynucleotide sequences encoding the targeted self-proteins or allergens (J. Immunol 162:3336-41; Curr. Dir. Autoimmun. 2:203-16). For all of these therapies, the antigens administered (or encoded in DNA) for purposes of immune suppression may comprise all or a portion of the epitopes identified by the array of the present invention.

The assay of the present invention may also be used to assess the efficacy of a pharmaceutical agent for individually treating an inflammation associated disease, wherein an anti-inflammatory activity above a predetermined threshold is indicative of therapeutic efficacy of the pharmaceutical agent.

According to this aspect of the present invention, the predetermined threshold may be selected as being at most 90%, more preferably 70%, more preferably 50%, more preferably 20% and even more preferably 10% or less the inflammatory activity than when no pharmaceutical agent is added.

Following selection of an individually optimized drug treatment, the chosen pharmaceutical agent may be used to treat an inflammation associated disorder.

As used herein the term “treating” refers to the prevention of some or all of the symptoms associated with an inflammation associated disease, a condition or disorder. The term “treating” also refers to alleviating the symptoms or underlying cause of an inflammation associated disease, prolongation of life expectancy of patients having a disease, as well as complete recovery from a disease.

It is envisaged by the present invention that the drug treatment is optimized several times during the course of a disease for a particular subject.

Examples of inflammation associated diseases and disorders are summarized infra.

Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander RB. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala 0. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov DG. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander RB. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra HS. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al. Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyperacute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphoctyic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

It is expected that during the life of this patent many relevant treatments will be developed and autoantigens discovered and the scope of the terms pharmaceutical agent and autoantigen is intended to include all such new technologies a priori.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Selection of Appropriate Concentrations of Immunomodulatory Drugs for the Immunomodulatory Drags Matching Method (IDMM)

All immunomodulatory drugs used for the treatment of MS patients are known to reduce pro-inflammatory cytokine production and specifically TNF-α. For example, the secretion of TNF-α in a short-term peripheral blood mononuclear cell (PBMC) culture is inhibited by the presence of an immunomodulatory drug. In order to design a method for comparing immunomodulatory drugs for the treatment of MS, PBMCs were specifically stimulated by an MS autoantigen, the encephalitogenic peptide, myelin oligodendrocyte glycoprotein (MOG) so as to induce over-expression of autoreactive cells specific for MS. MS immunomodulatory drugs were added to the cell cultures and those that showed the highest inhibition of TNF-α secretion were selected to treat the patient from where the cells were derived.

As various immunomodulatory drugs are formulated differently and are delivered by different routes of administration, appropriate drug concentrations were established for the IDMM. The in-vitro concentration for each drug was calculated according to the serum levels acquired after in-vivo injections or according to functional equivalent tests. The drug concentrations used in the IDDM are provided below in Table 1.

TABLE 1

| | No Drug | Rebif | Avonex | Betaferon | Copaxone | IVIg |
|---|---|---|---|---|---|---|
| PBMC | 2.5 × 10⁵ | 2.5 × 10⁵ | 2.5 × 10⁵ | 2.5 × 10⁵ | 2.5 × 10⁵ | 2.5 × 10⁵ |
| MOG(mg/ml) | 15 | 15 | 15 | 15 | 15 | 15 |
| Treatment | | 40 U/ml | 25 U/ml | 20 U/ml | 10 mkg/ml | 2.5 mg/ml |

Specifically, the concentration of IFN-b-1a (Avonex™) was selected by analyzing a pharmacokinetic study of Avonex™ which demonstrated that mean serum activities in healthy volunteers following IM administration of 6 MIU reached 25 U/ml (Alam J., 1997, Pharm Res, 14:546-549). This concentration was selected for the IDDM.

The concentration of IFN-b-1 a (Rebif™) was selected by analyzing a pharmacokinetic and pharmacodynamic study of Rebif™ which investigated the effect of Rebif™ in healthy volunteers. The study demonstrated that an injection of 6 MIU (22 μg) inhibits mitogen-induced PBMC TNF-α production by 8-13% (dependent on the mitogen), and three times per week demonstrated suppression by 29-38% (Rothuizen L., J Neuroimmunol. 1999 Sep. 1; 99(1):131-41). A concentration of 40 U/ml was selected for IDDM. This suppressed TNF-α secretion by a similar amount to three injections/week (between 29-38%).

The concentration of IFN-b-1b (Betaferon™) was selected by analyzing the drug serum concentration following either single or multiple subcutaneous injections. An 8 MIU serum concentration was 20 U/ml (Schering Interferon beta-1b Betaferon® Betaseron®. Investigator's Brochure, 4.0/22 Oct. 2001).

The immunomodulatory properties of in-vitro immunoglobulins are dose-dependent (Reske D., Acta Neurol Scand 2003:108, 267-273). It was demonstrated that in vivo serum TNF-α inhibition after standard single IV administration resulted in a 50% stable reduction from baseline secretion level. In-vitro experiments demonstrated that the same inhibition level was reached using 2.5 mg/ml of IVIg.

The concentration of Glatiramer acetate (Copaxone™) was selected according to the study of Weber M. S. et al., 2004 (Brain, 2004, 127: 1370-1378). The in-vitro concentration of 10 μg/ml was selected as it reached the same inhibitory effect on TNF-α producing cells as the in-vivo experiments.

### Example 2

### Clinical Study to Evaluate the Accuracy of the IDDM of the Present Invention

Patient criteria: Patients with clinically defined RRMS and free of immunomodulatory treatments and/or steroids for at least 30 days prior to blood withdrawal were included in the study.

Materials and Methods

15 cc of blood was withdrawn from each patient. PBMC (10⁶/ml) were separated from whole blood using a ficol-hypaque gradient, washed and seeded in 96-well Costar plate at a density of 2.5×10⁵ cells/well in 200 μl of complete RPMI 1640 containing 10% CCS and gentamicin/penicillin/streptamicin (Gibco, Grand Island, N.Y.). Stimulation of PBMC was performed for 48 hours in the presence of a commercially available synthetic encephalitogenic immunodominant peptides MOG34-56 (IGPRHPIRALVGDEVELPCRI, 15 μg/ml) (SEQ ID NO:1) as was used for stimulation autologous T cell lines (Achiron, 2004 Clin. Immunol. 2004, 113(2), 155-60).

Thereafter, cells were cultured in the presence of each immunomodulatory drug, e.g., interferon beta-1a (Avonex™ or Rebif™), interferon beta-1b (Betaferon™), Glatiramer acetate (Copaxone™), or IVIg in adjusted therapeutic concentrations (see Table 1 above).

Following a 48 hour incubation, TNF-α levels were measured in the supernatants by ELISA using commercial kits (R&D).

Results

Patient statistics: IDMM was applied to 54 relapsing-remitting MS patients (38 females).

Mean±SE, Age: 42.2±1.5 year (y)
Disease duration: 7.7±1.0 y
Expanded Disability Status Scale (EDSS): 3.1±0.3
Annual relapse rate: 1.26±0.28

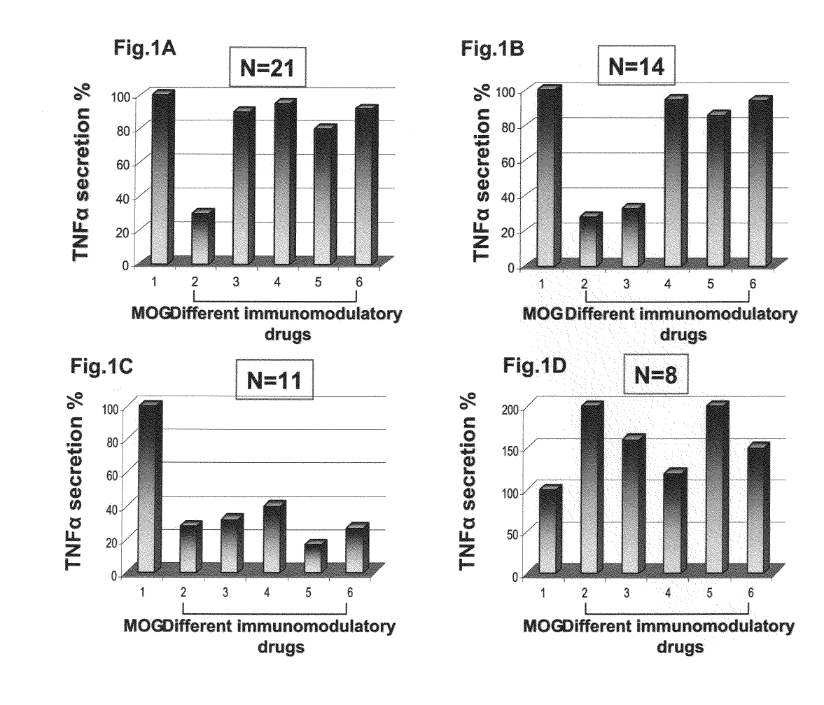

IDMM Results: Four possible IDMM results were obtained by comparing TNF-α levels in the supernatant of MOG stimulated PBMC without any drug. FIG. 1A is a bar graph illustrating the response of a typical patient which showed a positive response to one particular drug (in this case drug 2). Altogether, 21 patients (38% of total patients tested) responded in a similar way, although the particular drug varies amongst the patients. FIG. 1B shows the response of a typical patient which showed a positive response to two particular drugs (in this case, drugs 2 and 3). Altogether 14 patients (26% of total patients) responded in a similar way, although the particular two drugs varied amongst the patients. FIG. 1C illustrates the response of a typical patient which showed a positive response to all the drugs tested. Altogether 11 patients (20% of total patients) showed a positive response to all the drugs. FIG. 1D illustrates the response of a typical patient which showed a negative response to the tested drugs. Altogether, 8 patients (15% of total patients) showed a negative response to the drugs. Indeed, MOG stimulated TNFα levels without drugs were lower than with any of the immunomodulatory drugs. Thus the IDMM was not informative for selection of a drug for these patients and were not included in the study. Accordingly, in 46 patients (85%) an informative response was detected. Following the IDMM, 36 patients received an immunomodulatory drug according to the test (matched group), while 10 patients received treatment different from the test results (non-matched group).

Short-term outcome: After short-term immunomodulatory treatment of 16 weeks the number of relapses was compared between groups. As shown in FIG. 2, in the matched group, 2.7% of patients (1/36) had an acute relapse, while in the non-matched group 40% of patients (4/10) developed a relapse, (p<0.001). Thus, selection of an immunomodulatory drug according to the IDMM of the present invention significantly reduced the number of patients having an acute relapse.

Long-term outcome: 41 patients (mean±SE, age 41.7±1.7 years, disease duration 7.0±1.2 years, EDSS 3.5±0.3), completed one year of clinical follow-up. 30 patients received an immunomodulatory drug in accordance to the IDMM results (matched group), and 11 patients received a different immunomodulatory treatment from the test results (non-matched group).

The number of relapses (FIG. 3) and the time to next relapse (FIG. 4) were compared between groups. Relapse rate significantly decreased in the matched group from 1.4±0.3 to 0.5±0.1, p<0.01, while in the non-matched group no significant change in relapse rate occurred (1.3±0.3 and 0.9±0.3, p=0.22), suggesting lower efficacy of treatment. The time between relapses was 222±29 days in the matched group and 180±21 days in the non-matched group, p=0.1. Though this result was not significant statistically it demonstrates a trend towards an increased time until the next relapse in the matched IDMM group.

### Example 3

### Individual Tailoring of IDMM by Myelin Protein Immunogenic Epitopes

Several myelin-associated proteins have been identified as auto-antigens in MS including Myelin-associated Glycoprotein (MAG), Myelin-oligodendrocyte Glycoprotein (MOG), Myelin Basic Protein (MBP), Proteolipid Protein (PLP) and other minor proteins.

T-cells are activated by specific peptide epitopes that are determined within the antigen processing pathways and presented on the surface of other cells bound to MHC molecules. For more precise performing of the IDMM procedure described in Example 1 and 2, the specific epitope that stimulates T-cells in a particular patient could be evaluated.

Generation of a database of unique short peptide sequences which include all possible myelin epitopes: A method for comprehensive screening of T cell epitopes within the myelin protein family was performed essentially as described by Beiβbarth T et al., (Bioinformatics, 2005, Vol. 21 Suppl, i29-i37). A set of unique short peptides sequences was generated that included all possible myelin epitopes using an algorithm that takes into consideration that every individual has a pool of T cells, each with distinct T cell receptors relatively specific for 9-12 amino acid long sequences on antigen presenting cells. The algorithm selected a minimal number of 20 amino acid sequences that contain all-unique 12 amino acid sequences in the whole myelin protein family.

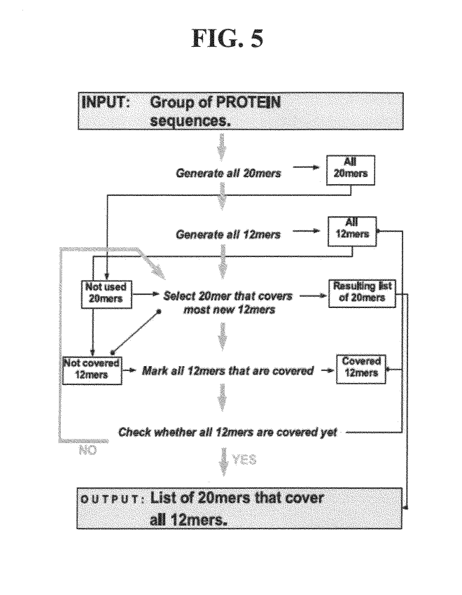

Initially a database was constructed into which amino-acid sequences of all myelin proteins (MBP, PLP, MOG, MAG, CNPase, crystallin, S100beta) were inserted into one file. 20 amino acid peptides containing all-unique 12 amino acid sequences occurring within all families of myelin proteins (MBP, PLP, MOG, MAG, CNPase, crystallin, S100beta) were screened. T-cell epitopes are expected to have a length of 9-12 amino acids. The number of all-unique 12 amino acid peptides would be too large, i.e. >15 000 and impractical for testing. Therefore, a set of 20 amino acid peptides was used that incorporates all 12 amino acid peptides (and therefore all 9 amino acid peptides as well) in myelin proteins, exploiting the opportunity that each 20 amino acid peptide can cover up to nine different 12 amino acid peptides. An iterative algorithm was used to compute such a set of peptides (FIG. 5). The algorithm starts with the protein sequences of a group of unaligned proteins from a protein family and generates all unique overlapping 20 amino acid and 12 amino acid peptides. It selects 20 amino acid peptides until a set is selected that covers all unique 12 amino acid peptides.

Results

A total of 214 amino-acid sequences represented all myelin proteins with their isoforms were used as input. After filtering overlapping peptides, 58 unique sequences were left for the above described analysis. From this 3751, unique 20 amino acid peptides and 3673 12 amino acid peptides were generated. Finally a minimal list of 435 20 amino acid peptides that included all possible 12 amino acid peptides was established (Table 2).

TABLE 2

| Unique epitope | |
|---|---|
| sequence/ | |
| SEQ ID NO: | Description |
| rsrgaeatesklsggfrwtq | myelin associated glycopro- |
| SEQ ID NO: 2 | tein Myelin associated glyco- |
| | protein isoform a variant. |
| | (Q59GD9 661 aa linear PRI 10- |
| | MAY-2005) |
| sklsggftwtqktsptqgds | myelin associated glycopro- |
| SEQ ID NO: 3 | tein Myelin associated glyco- |
| | protein isoform a variant. |
| | (Q59GD9 661 aa linear PRI 10- |
| | MAY-2005) |
| tqktsptqgdsaithslyrm | myelin associated glycopro- |
| SEQ ID NO: 4 | tein Myelin associated glyco- |
| | protein isoform a variant. |
| | (Q59GD9 661 aa linear PRI 10- |
| | MAY-2005) |
| dsaithslyrmifltalplf | myelin associated glycopro- |
| SEQ ID NO: 5 | tein Myelin associated glyco- |
| | protein isoform a variant. |
| | (Q59GD9 661 aa linear PRI 10- |
| | MAY-2005) |
| rmifltalplfwimisasrg | myelin associated glycopro- |
| SEQ ID NO: 6 | tein Myelin associated glyco- |
| | protein isoform a variant. |
| | (Q59GD9 661 aa linear PRI 10- |
| | MAY-2005) |
| lfwimisasrgghwgawmps | myelin associated glycopro- |
| SEQ ID NO: 7 | tein Myelin associated glyco- |
| | protein isoform a variant. |
| | (Q59GD9 661 aa linear PRI 10- |
| | MAY-2005) |
| rgghwgawmpssisafegtc | myelin associated glycopro- |
| SEQ ID NO: 8 | tein Myelin associated glyco- |
| | protein isoform a variant. |
| | (Q59GD9 661 aa linear PRI 10- |
| | MAY-2005) |
| pssisafegtcvsipcrfdf | myelin associated glycopro- |
| SEQ ID NO: 9 | tein Myelin associated glyco- |
| | protein isoform a variant. |
| | (Q59GD9 661 aa linear PRI 10- |
| | MAY-2005) |
| tcvsipcrfdfpdelrpavv | myelin associated glycopro- |
| SEQ ID NO: 10 | tein Myelin associated glyco- |
| | protein isoform a variant. |
| | (Q59GD9 661 aa linear PRI 10- |
| | MAY-2005) |
| dfpdelrpavvhgvwyfnsp | myelin associated glycopro- |
| SEQ ID NO: 11 | tein Myelin associated glyco- |
| | protein isoform a variant. |
| | (Q59GD9 661 aa linear PRI 10- |
| | MAY-2005) |
| vvhgvwyfnspypknyppvv | myelin associated glycopro- |
| SEQ ID NO: 12 | tein Myelin associated glyco- |
| | protein isoform a variant. |
| | (Q59GD9 661 aa linear PRI 10- |
| | MAY-2005) |
| spypknyppvvtksrtqvvh | myelin associated glycopro- |
| SEQ ID NO: 13 | tein Myelin associated glyco- |
| | protein isoform a variant. |
| | (Q59GD9 661 aa linear PRI 10- |
| | MAY-2005) |
| vvfksrtqvvhesfqgrsrl | myelin associated glycopro- |
| SEQ ID NO: 14 | tein Myelin associated glyco- |
| | protein isoform a variant. |
| | (Q59GD9 661 aa linear PRI 10- |
| | MAY-2005) |
| vhesfqgrsrllgdlglrnc | myelin associated glycopro- |
| SEQ ID NO: 15 | tein Myelin associated glyco- |
| | protein isoform a variant. |
| | (Q59GD9 661 aa linear PRI 10- |
| | MAY-2005) |
| rllgdlglrnctlllsnvsp | myelin associated glycopro- |
| SEQ ID NO: 16 | tein Myelin associated glyco- |
| | protein isoform a variant. |
| | (Q59GD9 661 aa linear PRI 10- |
| | MAY-2005) |
| nctlllsnvspelggkyyfr | myelin associated glycopro- |
| SEQ ID NO: 17 | tein Myelin associated glyco- |
| | protein isoform a variant. |
| | (Q59GD9 661 aa linear PRI 10- |
| | MAY-2005) |
| spelggkyyfrgdlggynqy | myelin associated glycopro- |
| SEQ ID NO: 18 | tein Myelin associated glyco- |
| | protein isoform a variant. |
| | (Q59GD9 661 aa linear PRI 10- |
| | MAY-2005) |
| frgdlggynqytfsehsvld | myelin associated glycopro- |
| SEQ ID NO: 19 | tein Myelin associated glyco- |
| | protein isoform a variant. |
| | (Q59GD9 661 aa linear PRI 10- |
| | MAY-2005) |
| qytfsehsvldivntpnivv | myelin associated glycopro- |
| SEQ ID NO: 20 | tein Myelin associated glyco- |
| | protein isoform a variant. |
| | (Q59GD9 661 aa linear PRI 10- |
| | MAY-2005) |
| ldivntpnivvppevvagte | myelin associated glycopro- |
| SEQ ID NO: 21 | tein Myelin associated glyco- |
| | protein isoform a variant. |
| | (Q59GD9 661 aa linear PRI 10- |
| | MAY-2005) |
| vvppevvagtevevscmvpd | myelin associated glycopro- |
| SEQ ID NO: 22 | tein Myelin associated glyco- |
| | protein isoform a variant. |
| | (Q59GD9 661 aa linear PRI 10- |
| | MAY-2005) |
| tevevscmvpdncpelrpel | myelin associated glycopro- |
| SEQ ID NO: 23 | tein Myelin associated glyco- |
| | protein isoform a variant. |
| | (Q59GD9 661 aa linear PRI 10- |
| | MAY-2005) |
| pdncpelrpelswlgheglg | myelin associated glycopro- |
| SEQ ID NO: 24 | tein Myelin associated glyco- |
| | protein isoform a variant. |
| | (Q59GD9 661 aa linear PRI 10- |
| | MAY-2005) |
| elswlgheglgepavlgrlr | myelin associated glycopro- |
| SEQ ID NO: 25 | tein Myelin associated glyco- |
| | protein isoform a variant. |
| | (Q59GD9 661 aa linear PRI 10- |
| | MAY-2005) |
| lgepavlgrlredegtwvqv | myelin associated glycopro- |
| SEQ ID NO: 26 | tein Myelin associated glyco- |
| | protein isoform a variant. |
| | (Q59GD9 661 aa linear PRI 10- |
| | MAY-2005) |
| Lledegtwvqvsllhfvptr | myelin associated glycopro- |
| SEQ ID NO: 27 | tein Myelin associated glyco- |
| | protein isoform a variant. |
| | (Q59GD9 661 aa linear PRI 10- |
| | MAY-2005) |
| qvsllhfvptreanghrlgc | myelin associated glycopro- |
| SEQ ID NO: 28 | tein Myelin associated glyco- |
| | protein isoform a variant. |
| | (Q59GD9 661 aa linear PRI 10- |
| | MAY-2005) |
| treanghrlgcqasfpnttl | myelin associated glycopro- |
| SEQ ID NO: 29 | tein Myelin associated glyco- |
| | protein isoform a variant. |
| | (Q59GD9 661 aa linear PRI 10- |
| | MAY-2005) |
| gcqasfpnttlqfegyasmd | myelin associated glycopro- |
| SEQ ID NO: 30 | tein Myelin associated glyco- |
| | protein isoform a variant. |
| | (Q59GD9 661 aa linear PRI 10- |
| | MAY-2005) |
| tlqfegyasmdvkyppvive | myelin associated glycopro- |
| SEQ ID NO: 31 | tein Myelin associated glyco- |
| | protein isoform a variant. |
| | (Q59GD9 661 aa linear PRI 10- |
| | MAY-2005) |
| mdvkyppvivemnssveaie | myelin associated glycopro- |
| SEQ ID NO: 32 | tein Myelin associated glyco- |
| | protein isoform a variant. |
| | (Q59GD9 661 aa linear PRI 10- |
| | MAY-2005) |
| vemnssveaiegshvsllcg | myelin associated glycopro- |
| SEQ ID NO: 33 | tein Myelin associated glyco- |
| | protein isoform a variant. |
| | (Q59GD9 661 aa linear PRI 10- |
| | MAY-2005) |
| iegshvsllcgadsnpppll | myelin associated glycopro- |
| SEQ ID NO: 34 | tein Myelin associated glyco- |
| | protein isoform a variant. |
| | (Q59GD9 661 aa linear PRI 10- |
| | MAY-2005) |
| cgadsnppplltwmrdgtvl | myelin associated glycopro- |
| SEQ ID NO: 35 | tein Myelin associated glyco- |
| | protein isoform a variant. |
| | (Q59GD9 661 aa linear PRI 10- |
| | MAY-2005) |
| lltwmrdgtvlreavaesll | myelin associated glycopro- |
| SEQ ID NO: 36 | tein Myelin associated glyco- |
| | protein isoform a variant. |
| | (Q59GD9 661 aa linear PRI 10- |
| | MAY-2005) |
| vlreavaesllleleevtpa | myelin associated glycopro- |
| SEQ ID NO: 37 | tein Myelin associated glyco- |
| | protein isoform a variant. |
| | (Q59GD9 661 aa linear PRI 10- |
| | MAY-2005) |
| llleleevtpaedgvyacla | myelin associated glycopro- |
| SEQ ID NO: 38 | tein Myelin associated glyco- |
| | protein isoform a variant. |
| | (Q59GD9 661 aa linear PRI 10- |
| | MAY-2005) |
| paedgvyaclaenaygqdrr | myelin associated glycopro- |
| SEQ ID NO: 39 | tein Myelin associated glyco- |
| | protein isoform a variant. |
| | (Q59GD9 661 aa linear PRI 10- |
| | MAY-2005) |
| laenaygqdnrtvglsvmya | myelin associated glycopro- |
| SEQ ID NO: 40 | tein Myelin associated glyco- |
| | protein isoform a variant. |
| | (Q59GD9 661 aa linear PRI 10- |
| | MAY-2005) |
| nrtvglsvmyapwkptvngt | myelin associated glycopro- |
| SEQ ID NO: 41 | tein Myelin associated glyco- |
| | protein isoform a variant. |
| | (Q59GD9 661 aa linear PRI 10- |
| | MAY-2005) |
| yapwkptvngtmvavegetv | myelin associated glycopro- |
| SEQ ID NO: 42 | tein Myelin associated glyco- |
| | protein isoform a variant. |
| | (Q59GD9 661 aa linear PRI 10- |
| | MAY-2005) |
| gtmvavegetvsilcstqsn | myelin associated glycopro- |
| SEQ ID NO: 43 | tein Myelin associated glyco- |
| | protein isoform a variant. |
| | (Q59GD9 661 aa linear PRI 10- |
| | MAY-2005) |
| tvsilcstqsnpdpiltifk | myelin associated glycopro- |
| SEQ ID NO: 44 | tein Myelin associated glyco- |
| | protein isoform a variant. |
| | (Q59GD9 661 aa linear PRI 10- |
| | MAY-2005) |
| snpdpiltifkekqilstvi | myelin associated glycopro- |
| SEQ ID NO: 45 | tein Myelin associated glyco- |
| | protein isoform a variant. |
| | (Q59GD9 661 aa linear PRI 10- |
| | MAY-2005) |
| fkekqilstviyeselqlel | myelin associated glycopro- |
| SEQ ID NO: 46 | tein Myelin associated glyco- |
| | protein isoform a variant. |
| | (Q59GD9 661 aa linear PRI 10- |
| | MAY-2005) |
| viyeselqlelpavspeddg | myelin associated glycopro- |
| SEQ ID NO: 47 | tein Myelin associated glyco- |
| | protein isoform a variant. |
| | (Q59GD9 661 aa linear PRI 10- |
| | MAY-2005) |
| elpavspeddgeywcvaenq | myelin associated glycopro- |
| SEQ ID NO: 48 | tein Myelin associated glyco- |
| | protein isoform a variant. |
| | (Q59GD9 661 aa linear PRI 10- |
| | MAY-2005) |
| dgeywcvaenqygqratafn | myelin associated glycopro- |
| SEQ ID NO: 49 | tein Myelin associated glyco- |
| | protein isoform a variant. |
| | (Q59GD9 661 aa linear PRI 10- |
| | MAY-2005) |
| nqygqratafnlsvefapvl | myelin associated glycopro- |
| SEQ ID NO: 50 | tein Myelin associated glyco- |
| | protein isoform a variant. |
| | (Q59GD9 661 aa linear PRI 10- |
| | MAY-2005) |
| fnlsvefapvllleshcaaa | myelin associated glycopro- |
| SEQ ID NO: 51 | tein Myelin associated glyco- |
| | protein isoform a variant. |
| | (Q59GD9 661 aa linear PRI 10- |
| | MAY-2005) |
| vllleshcaaardtvqclcv | myelin associated glycopro- |
| SEQ ID NO: 52 | tein Myelin associated glyco- |
| | protein isoform a variant. |
| | (Q59GD9 661 aa linear PRI 10- |
| | MAY-2005) |
| aardtvqclcvvksnpepsv | myelin associated glycopro- |
| SEQ ID NO: 53 | tein Myelin associated glyco- |
| | protein isoform a variant. |
| | (Q59GD9 661 aa linear PRI 10- |
| | MAY-2005) |
| cvvksnpepsvafelpsrnv | myelin associated glycopro- |
| SEQ ID NO: 54 | tein Myelin associated glyco- |
| | protein isoform a variant. |
| | (Q59GD9 661 aa linear PRI 10- |
| | MAY-2005) |
| svafelpsrnvtvneseref | myelin associated glycopro- |
| SEQ ID NO: 55 | tein Myelin associated glyco- |
| | protein isoform a variant. |
| | (Q59GD9 661 aa linear PRI 10- |
| | MAY-2005) |
| nvtvneserefvysersglv | myelin associated glycopro- |
| SEQ ID NO: 56 | tein Myelin associated glyco- |
| | protein isoform a variant. |
| | (Q59GD9 661 aa linear PRI 10- |
| | MAY-2005) |
| efvysersglvltsiltlrg | myelin associated glycopro- |
| SEQ ID NO: 57 | tein Myelin associated glyco- |
| | protein isoform a variant. |
| | (Q59GD9 661 aa linear PRI 10- |
| | MAY-2005) |
| lvltsiltlrgqaqapprvi | myelin associated glycopro- |
| SEQ ID NO: 58 | tein Myelin associated glyco- |
| | protein isoform a variant. |
| | (Q59GD9 661 aa linear PRI 10- |
| | MAY-2005) |
| rgqaqapprvictarnlyga | myelin associated glycopro- |
| SEQ ID NO: 59 | tein Myelin associated glyco- |
| | protein isoform a variant. |
| | (Q59GD9 661 aa linear PRI 10- |
| | MAY-2005) |
| victarnlygakslelpfqg | myelin associated glycopro- |
| SEQ ID NO: 60 | tein Myelin associated glyco- |
| | protein isoform a variant. |
| | (Q59GD9 661 aa linear PRI 10- |
| | MAY-2005) |
| gakslelpfqgahrlmwaki | myelin associated glycopro- |
| SEQ ID NO: 61 | tein Myelin associated glyco- |
| | protein isoform a variant. |
| | (Q59GD9 661 aa linear PRI 10- |
| | MAY-2005) |
| qgahrlmwakigpvgavvaf | myelin associated glycopro- |
| SEQ ID NO: 62 | tein Myelin associated glyco- |
| | protein isoform a variant. |
| | (Q59GD9 661 aa linear PRI 10- |
| | MAY-2005) |
| kigpvgavvafailiaivcy | myelin associated glycopro- |
| SEQ ID NO: 63 | tein Myelin associated glyco- |
| | protein isoform a variant. |
| | (Q59GD9 661 aa linear PRI 10- |
| | MAY-2005) |
| afailiaivcyitqtrrkkn | myelin associated glycopro- |
| SEQ ID NO: 64 | tein Myelin associated glyco- |
| | protein isoform a variant. |
| | (Q59GD9 661 aa linear PRI 10- |
| | MAY-2005) |
| cyitqtrrkknvtespsfsa | myelin associated glycopro- |
| SEQ ID NO: 65 | tein Myelin associated glyco- |
| | protein isoform a variant. |
| | (Q59GD9 661 aa linear PRI 10- |
| | MAY-2005) |
| knvtespsfsagdnppvlfs | myelin associated glycopro- |
| SEQ ID NO: 66 | tein Myelin associated glyco- |
| | protein isoform a variant. |
| | (Q59GD9 661 aa linear PRI 10- |
| | MAY-2005) |
| sagdnppvlfssdfrisgap | myelin associated glycopro- |
| SEQ ID NO: 67 | tein Myelin associated glyco- |
| | protein isoform a variant. |
| | (Q59GD9 661 aa linear PRI 10- |
| | MAY-2005) |
| fssdfrisgapekyeserrl | myelin associated glycopro- |
| SEQ ID NO: 68 | tein Myelin associated glyco- |
| | protein isoform a variant. |
| | (Q59GD9 661 aa linear PRI 10- |
| | MAY-2005) |
| apekyeserrlgserrllgl | myelin associated glycopro- |
| SEQ ID NO: 69 | tein Myelin associated glyco- |
| | protein isoform a variant. |
| | (Q59GD9 661 aa linear PRI 10- |
| | MAY-2005) |
| rlgserrllglrgeppeldl | myelin associated glycopro- |
| SEQ ID NO: 70 | tein Myelin associated glyco- |
| | protein isoform a variant. |
| | (Q59GD9 661 aa linear PRI 10- |
| | MAY-2005) |
| glrgeppeldlsyshsdlgk | myelin associated glycopro- |
| SEQ ID NO: 71 | tein Myelin associated glyco- |
| | protein isoform a variant. |
| | (Q59GD9 661 aa linear PRI 10- |
| | MAY-2005) |
| dlsyshsdlgkrptkdsytl | myelin associated glycopro- |
| SEQ ID NO: 72 | tein Myelin associated glyco- |
| | protein isoform a variant. |
| | (Q59GD9 661 aa linear PRI 10- |
| | MAY-2005) |
| gkrptkdsytlteelaeccq | myelin associated glycopro- |
| SEQ ID NO: 73 | tein Myelin associated glyco- |
| | protein isoform a variant. |
| | (Q59GD9 661 aa linear PRI 10- |
| | MAY-2005) |
| divntpnivvplevvagtev | myelin associated glycopro- |
| SEQ ID NO: 74 | tein Myelin associated glyco- |
| | protein isoform a variant. |
| | (Q53HD1 626 aa linear PRI 13- |
| | SEP.-2005) |
| mdvkyppvivevnssveaie | myelin associated glycopro- |
| SEQ ID NO: 75 | tein Myelin associated glyco- |
| | protein isoform a variant. |
| | (Q53HD1 626 aa linear PRI 13- |
| | SEP.-2005) |
| lelpfqgahrltwakigpvg | myelin associated glycopro- |
| SEQ ID NO: 76 | tein Myelin associated glyco- |
| | protein isoform a variant. |
| | (Q53HD1 626 aa linear PRI 13- |
| | SEP.-2005) |
| nrtvglsvmyaswkptvngt | myelin associated glycopro- |
| SEQ ID NO: 77 | tein Myelin associated glyco- |
| | protein isoform a variant. |
| | (Q53HA1 626 aa linear PRI 13- |
| | SEP.-200) |
| dlsyshsdlgkqptkdsytl | myelin associated glycopro- |
| SEQ ID NO: 78 | tein Myelin associated glyco- |
| | protein isoform a variant. |
| | (Q53ES7 626 aa linear PRI 13- |
| | SEP.-2005) |
| risgapekyeskevstlesh | myelin associated glycopro- |
| SEQ ID NO: 79 | tein Myelin associated glyco- |
| | protein, isoform b. |
| | (Q567S4 582 aa linear PRI 10- |
| | MAY-2005) |
| meyqilkmslctfillfltp | Myelin-oligodendrocyte |
| SEQ ID NO: 80 | glycoprotein |
| | (NP_002535 440 aa linear PRI |
| | 24-SEP.-2005) |
| lclfillfltpgilcicplq | Myelin-oligodendrocyte |
| SEQ ID NO: 81 | glycoprotein |
| | (NP_002535 440 aa linear PRI |
| | 24-SEP.-2005) |
| tpgilcicplqcicterhrh | Myelin-oligodendrocyte |
| SEQ ID NO: 82 | glycoprotein |
| | (NP_002535 440 aa linear PRI |
| | 24-SEP.-2005) |
| lqcicterhrhvdcsgrnls | Myelin-oligodendrocyte |
| SEQ ID NO: 83 | glycoprotein |
| | (NP_002535 440 aa linear PRI |
| | 24-SEP.-2005) |
| rhvdcsgrnlstlpsglqen | Myelin-oligodendrocyte |
| SEQ ID NO: 84 | glycoprotein |
| | (NP_002535 440 aa linear PRI |
| | 24-SEP.-2005) |
| lstlpsglqeniihlnlsyn | Myelin-oligodendrocyte |
| SEQ ID NO: 85 | glycoprotein |
| | (NP_002535 440 aa linear PRI |
| | 24-SEP.-2005) |
| eniihlnlsynhftdlhnql | Myelin-oligodendrocyte |
| SEQ ID NO: 86 | glycoprotein |
| | (NP_002535 440 aa linear PRI |
| | 24-SEP.-2005) |
| ynhftdlhnqltqytnlrtl | Myelin-oligodendrocyte |
| SEQ ID NO: 87 | glycoprotein |
| | (NP_002535 440 aa linear PRI |
| | 24-SEP.-2005) |
| qltqytnlrtldisnnrles | Myelin-oligodendrocyte |
| SEQ ID NO: 88 | glycoprotein |
| | (NP_002535 440 aa linear PRI |
| | 24-SEP.-2005) |
| tldisnnrleslpahlprsl | Myelin-oligodendrocyte |
| SEQ ID NO: 89 | glycoprotein |
| | (NP_002535 440 aa linear PRI |
| | 24-SEP.-2005) |
| eslpahlprslwnmsaannn | Myelin-oligodendrocyte |
| SEQ ID NO: 90 | glycoprotein |
| | (NP_002535 440 aa linear PRI |
| | 24-SEP.-2005) |
| slwnmsaannniklldksdt | Myelin-oligodendrocyte |
| SEQ ID NO: 91 | glycoprotein |
| | (NP_002535 440 aa linear PRI |
| | 24-SEP.-2005) |
| nniklldksdtayqwnlkyl | Myelin-oligodendrocyte |
| SEQ ID NO: 92 | glycoprotein |
| | (NP_002535 440 aa linear PRI |
| | 24-SEP.-2005) |
| dtayqwnlkyldvsknmlek | Myelin-oligodendrocyte |
| SEQ ID NO: 93 | glycoprotein |
| | (NP_002535 440 aa linear PRI |
| | 24-SEP.-2005) |
| yldvsknmlekvvlikntlr | Myelin-oligodendrocyte |
| SEQ ID NO: 94 | glycoprotein |
| | (NP_002535 440 aa linear PRI |
| | 24-SEP.-2005) |
| ekvvlikntlrslevlnlss | Myelin-oligodendrocyte |
| SEQ ID NO: 95 | glycoprotein |
| | (NP_002535 440 aa linear PRI |
| | 24-SEP.-2005) |
| lrslevlnlssnklwtvptn | Myelin-oligodendrocyte |
| SEQ ID NO: 96 | glycoprotein |
| | (NP_002535 440 aa linear PRI |
| | 24-SEP.-2005) |
| ssnklwtvptnmpsklhivd | Myelin-oligodendrocyte |
| SEQ ID NO: 97 | glycoprotein |
| | (NP_002535 440 aa linear PRI |
| | 24-SEP.-2005) |
| tnmpsklhivdlsnnsltqi | Myelin-oligodendrocyte |
| SEQ ID NO: 98 | glycoprotein |
| | (NP_002535 440 aa linear PRI |
| | 24-SEP.-2005) |
| vdlsnnsltqilpgtlinlt | Myelin-oligodendrocyte |
| SEQ ID NO: 99 | glycoprotein |
| | (NP_002535 440 aa linear PRI |
| | 24-SEP.-2005) |
| qilpgtlinltnlthlylhn | Myelin-oligodendrocyte |
| SEQ ID NO: 100 | glycoprotein |
| | (NP_002535 440 aa linear PRI |
| | 24-SEP.-2005) |
| ltnlthlylhnnkftfipdq | Myelin-oligodendrocyte |
| SEQ ID NO: 101 | glycoprotein |
| | (NP_002535 440 aa linear PRI |
| | 24-SEP.-2005) |
| hnnkftfipdqsfdqlfqlq | Myelin-oligodendrocyte |
| SEQ ID NO: 102 | glycoprotein |
| | (NP_002535 440 aa linear PRI |
| | 24-SEP.-2005) |
| dqsfdqlfqlqeitlynnrw | Myelin-oligodendrocyte |
| SEQ ID NO: 103 | glycoprotein |
| | (NP_002535 440 aa linear PRI |
| | 24-SEP.-2005) |
| lqeitlynnrwscdhkqnit | Myelin-oligodendrocyte |
| SEQ ID NO: 104 | glycoprotein |
| | (NP_002535 440 aa linear PRI |
| | 24-SEP.-2005) |
| rwscdhkqnityllkwmmet | Myelin-oligodendrocyte |
| SEQ ID NO: 105 | glycoprotein |
| | (NP_002535 440 aa linear PRI |
| | 24-SEP.-2005) |
| ityllkwmmetkahvigtpc | Myelin-oligodendrocyte |
| SEQ ID NO: 106 | glycoprotein |
| | (NP_002535 440 aa linear PRI |
| | 24-SEP.-2005) |
| etkahvigtpcstqisslke | Myelin-oligodendrocyte |
| SEQ ID NO: 107 | glycoprotein |
| | (NP_002535 440 aa linear PRI |
| | 24-SEP.-2005) |
| pcstqisslkehnmyptpsg | Myelin-oligodendrocyte |
| SEQ ID NO: 108 | glycoprotein |
| | (NP_002535 440 aa linear PRI |
| | 24-SEP.-2005) |
| kehnmyptpsgftsslftvs | Myelin-oligodendrocyte |
| SEQ ID NO: 109 | glycoprotein |
| | (NP_002535 440 aa linear PRI |
| | 24-SEP.-2005) |
| sgftsslftvsgmqtvdtin | Myelin-oligodendrocyte |
| SEQ ID NO: 110 | glycoprotein |
| | (NP_002535 440 aa linear PRI |
| | 24-SEP.-2005) |
| vsgmqtvdtinslsvvtqpk | Myelin-oligodendrocyte |
| SEQ ID NO: 111 | glycoprotein |
| | (NP_002535 440 aa linear PRI |
| | 24-SEP.-2005) |
| inslsvvtqpkvtkipkqyr | Myelin-oligodendrocyte |
| SEQ ID NO: 112 | glycoprotein |
| | (NP_002535 440 aa linear PRI |
| | 24-SEP.-2005) |
| pkvtkipkqyrtkettfgat | Myelin-oligodendrocyte |
| SEQ ID NO: 113 | glycoprotein |
| | (NP_002535 440 aa linear PRI |
| | 24-SEP.-2005) |
| yrtkettfgatlskdttfts | Myelin-oligodendrocyte |
| SEQ ID NO: 114 | glycoprotein |
| | (NP_002535 440 aa linear PRI |
| | 24-SEP.-2005) |
| atlskdttflstdkafvpyp | Myelin-oligodendrocyte |
| SEQ ID NO: 115 | glycoprotein |
| | (NP_002535 440 aa linear PRI |
| | 24-SEP.-2005) |
| tstdkafvpypedtstetin | Myelin-oligodendrocyte |
| SEQ ID NO: 116 | glycoprotein |
| | (NP_002535 440 aa linear PRI |
| | 24-SEP.-2005) |
| ypedtstetinsheaaaatl | Myelin-oligodendrocyte |
| SEQ ID NO: 117 | glycoprotein |
| | (NP_002535 440 aa linear PRI |
| | 24-SEP.-2005) |
| insheaaaatltihlqdgmv | Myelin-oligodendrocyte |
| SEQ ID NO: 118 | glycoprotein |
| | (NP_002535 440 aa linear PRI |
| | 24-SEP.-2005) |
| tltihlqdgmvtntsltsst | Myelin-oligodendrocyte |
| SEQ ID NO: 119 | glycoprotein |
| | (NP_002535 440 aa linear PRI |
| | 24-SEP.-2005) |
| mvtntsltsstkssptpmtl | Myelin-oligodendrocyte |
| SEQ ID NO: 120 | glycoprotein |
| | (NP_002535 440 aa linear PRI |
| | 24-SEP.-2005) |
| stkssptpmtlsitsgmpnn | Myelin-oligodendrocyte |
| SEQ ID NO: 121 | glycoprotein |
| | (NP_002535 440 aa linear PRI |
| | 24-SEP.-2005) |
| tlsitsgmpnnfsempqqst | Myelin-oligodendrocyte |
| SEQ ID NO: 122 | glycoprotein |
| | (NP_002535 440 aa linear PRI |
| | 24-SEP.-2005) |
| nnfsempqqsttlnlwreet | Myelin-oligodendrocyte |
| SEQ ID NO: 123 | glycoprotein |
| | (NP_002535 440 aa linear PRI |
| | 24-SEP.-2005) |
| sttlnlwreetttnvktplp | Myelin-oligodendrocyte |
| SEQ ID NO: 124 | glycoprotein |
| | (NP_002535 440 aa linear PRI |
| | 24-SEP.-2005) |
| etttnvktplpsvanawkvn | Myelin-oligodendrocyte |
| SEQ ID NO: 125 | glycoprotein |
| | (NP_002535 440 aa linear PRI |
| | 24-SEP.-2005) |
| lpsvanawkvnasfllllnv | Myelin-oligodendrocyte |
| SEQ ID NO: 126 | glycoprotein |
| | (NP_002535 440 aa linear PRI |
| | 24-SEP.-2005) |
| mnrgfsrkshtflpkiffrk | CNPase (ACCESSION P09543) |
| SEQ ID NO: 127 | |
| htflpkiffrkmsssgakdk | CNPase (ACCESSION P09543) |
| SEQ ID NO: 128 | |
| rkmsssgakdkpelqfpflq | CNPase (ACCESSION P09543) |
| SEQ ID NO: 129 | |
| dkpelqfpflqdedtvatll | CNPase (ACCESSION P09543) |
| SEQ ID NO: 130 | |
| lqdedtvatllecktlfilr | CNPase (ACCESSION P09543) |
| SEQ ID NO: 131 | |
| llecktlfilrglpgsgkst | CNPase (ACCESSION P09543) |
| SEQ ID NO: 132 | |
| lrglpgsgkstlarvivdky | CNPase (ACCESSION P09543) |
| SEQ ID NO: 133 | |
| stlarvivdkyrdgtkmvsa | CNPase (ACCESSION P09543) |
| SEQ ID NO: 134 | |
| kyrdgtkmvsadaykitpga | CNPase (ACCESSION P09543) |
| SEQ ID NO: 135 | |
| sadaykitpgargafseeyk | CNPase (ACCESSION P09543) |
| SEQ ID NO: 136 | |
| gargafseeykrldedlaay | CNPase (ACCESSION P09543) |
| SEQ ID NO: 137 | |
| ykrldedlaaycrrrdiril | CNPase (ACCESSION P09543) |
| SEQ ID NO: 138 | |
| aycrrrdirilvlddtnher | CNPase (ACCESSION P09543) |
| SEQ ID NO: 139 | |
| ilvlddtnhererleqlfem | CNPase (ACCESSION P09543) |
| SEQ ID NO: 140 | |
| ererleqlfemadqyqyqvv | CNPase (ACCESSION P09543) |
| SEQ ID NO: 141 | |
| emadqyqyqvvlvepktawr | CNPase (ACCESSION P09543) |
| SEQ ID NO: 142 | |
| vvlvepktawrldcaqlkck | CNPase (ACCESSION P09543) |
| SEQ ID NO: 134 | |
| wrldcaqlkeknqwqlsadd | CNPase (ACCESSION P09543) |
| SEQ ID NO: 144 | |
| eknqwqlsaddlkklkpgle | CNPase (ACCESSION P09543) |
| SEQ ID NO: 145 | |
| ddkkklkpglekdflplyfg | CNPase (ACCESSION P09543) |
| SEQ ID NO: 146 | |
| lekdflplyfgwfltkksse | CNPase (ACCESSION P09543) |
| SEQ ID NO: 147 | |
| fgwfltkkssetlrkagqvf | CNPase (ACCESSION P09543) |
| SEQ ID NO: 148 | |
| setlrkagqvfleelgnhka | CNPase (ACCESSION P09543) |
| SEQ ID NO: 149 | |
| vfleelgnhkafkkelrqfv | CNPase (ACCESSION P09543) |
| SEQ ID NO: 150 | |
| kafkkelrqfvpgdeprekm | CNPase (ACCESSION P09543) |
| SEQ ID NO: 151 | |
| fvpgdeprekmdlvtyfgkr | CNPase (ACCESSION P09543) |
| SEQ ID NO: 152 | |
| kmdlvtyfgkrppgvlhctt | CNPase (ACCESSION P09543) |
| SEQ ID NO: 154 | |
| krppgvlhcttkfcdygkap | CNPase (ACCESSION P09543) |
| SEQ ID NO: 154 | |
| ttkfcdygkapgaeeyaqqd | CNPase (ACCESSION P09543) |
| SEQ ID NO: 155 | |
| apgaeeyaqqdvlkksyska | CNPase (ACCESSION P09543) |
| SEQ ID NO: 156 | |
| qdvlkksyskaftltisalf | CNPase (ACCESSION P09543) |
| SEQ ID NO: 157 | |
| kaftltisalfvtpkttgar | CNPase (ACCESSION P09543) |
| SEQ ID NO: 158 | |
| lfvtpkttgarvelseqqlq | CNPase (ACCESSION P09543) |
| SEQ ID NO: 159 | |
| arvelseqqlqlwpsdvdkl | CNPase (ACCESSION P09543) |
| SEQ ID NO: 160 | |
| lqlwpsdvdklsptdnlprg | CNPase (ACCESSION P09543) |
| SEQ ID NO: 161 | |
| klsptdnlprgsrahitlgc | CNPase (ACCESSION P09543) |
| SEQ ID NO: 162 | |
| rgsrahitlgcaadveavqt | CNPase (ACCESSION P09543) |
| SEQ ID NO: 163 | |
| gcaadveavqtgldlleilr | CNPase (ACCESSION P09543) |
| SEQ ID NO: 164 | |
| qtgldlleilrqekggsrge | CNPase (ACCESSION P09543) |
| SEQ ID NO: 165 | |
| lrqekggsrgeevgelsrgk | CNPase (ACCESSION P09543) |
| SEQ ID NO: 166 | |
| geevgelsrgklyslgngrw | CNPase (ACCESSION P09543) |
| SEQ ID NO: 167 | |
| gklyslgngrwmltlaknme | CNPase (ACCESSION P09543) |
| SEQ ID NO: 168 | |
| rwmltlaknmevraiftgyy | CNPase (ACCESSION P09543) |
| SEQ ID NO: 169 | |
| mevraiftgyygkgkpvptq | CNPase (ACCESSION P09543) |
| SEQ ID NO: 170 | |
| yygkgkpvptqgsrkggalq | CNPase (ACCESSION P09543) |
| SEQ ID NO: 171 | |
| maslsrpslpsclcsfllll | Myelin-oligodendrocyte glyco- |
| SEQ ID NO: 172 | protein Myelin oligodendro- |
| | cyte glycoprotein. |
| | (Q5STM2 321 aa linear PRI 13- |
| | SEP.-2005) |
| psclcsfllllllqvsssya | Myelin-oligodendrocyte glyco- |
| SEQ ID NO: 173 | protein Myelin oligodendro- |
| | cyte glycoprotein. |
| | (Q5STM2 321 aa linear PRI 13- |
| | SEP.-2005) |
| llllqvsssyagqfrvigpr | Myelin-oligodendrocyte glyco- |
| SEQ ID NO: 174 | protein Myelin oligodendro- |
| | cyte glycoprotein. |
| | (Q5STM2 321 aa linear PRI 13- |
| | SEP.-2005) |
| yagqfrvigprhpiralvgd | Myelin-oligodendrocyte glyco- |
| SEQ ID NO: 175 | protein Myelin oligodendro- |
| | cyte glycoprotein. |
| | (Q5STM2 321 aa linear PRI 13- |
| | SEP.-2005) |
| prhpiralvgdevelpcris | Myelin-oligodendrocyte glyco- |
| SEQ ID NO: 176 | protein Myelin oligodendro- |
| | cyte glycoprotein. |
| | (Q5STM2 321 aa linear PRI 13- |
| | SEP.-2005) |
| gdevelpcrispgknatgme | Myelin-oligodendrocyte glyco- |
| SEQ ID NO: 177 | protein Myelin oligodendro- |
| | cyte glycoprotein. |
| | (Q5STM2 321 aa linear PRI 13- |
| | SEP.-2005) |
| ispgknatgmevgwyrppfs | Myelin-oligodendrocyte glyco- |
| SEQ ID NO: 178 | protein Myelin oligodendro- |
| | cyte glycoprotein. |
| | (Q5STM2 321 aa linear PRI 13- |
| | SEP.-2005) |
| mevgwyrppfsrvvhlyrng | Myelin-oligodendrocyte glyco- |
| SEQ ID NO: 179 | protein Myelin oligodendro- |
| | cyte glycoprotein. |
| | (Q5STM2 321 aa linear PRI 13- |
| | SEP.-2005) |
| fsrvvhlymgkdqdgdqap | Myelin-oligodendrocyte glyco- |
| SEQ ID NO: 180 | protein Myelin oligodendro- |
| | cyte glycoprotein. |
| | (Q5STM2 321 aa linear PRI 13- |
| | SEP.-2005) |
| ngkdqdgdqapeyrgrtell | Myelin-oligodendrocyte glyco- |
| SEQ ID NO: 181 | protein Myelin oligodendro- |
| | cyte glycoprotein. |
| | (Q5STM2 321 aa linear PRI 13- |
| | SEP.-2005) |
| apeyrgrtellkdaigegkv | Myelin-oligodendrocyte glyco- |
| SEQ ID NO: 182 | protein Myelin oligodendro- |
| | cyte glycoprotein. |
| | (Q5STM2 321 aa linear PRI 13- |
| | SEP.-2005) |
| llkdaigegkvtlrirnvrf | Myelin-oligodendrocyte glyco- |
| SEQ ID NO: 183 | protein Myelin oligodendro- |
| | cyte glycoprotein. |
| | (Q5STM2 321 aa linear PRI 13- |
| | SEP.-2005) |
| kvtlrirnvrfsdeggftcf | Myelin-oligodendrocyte glyco- |
| SEQ ID NO: 184 | protein Myelin oligodendro- |
| | cyte glycoprotein. |
| | (Q5STM2 321 aa linear PRI 13- |
| | SEP.-2005) |
| rfsdeggftcffrdhsyqee | Myelin-oligodendrocyte glyco- |
| SEQ ID NO: 185 | protein Myelin oligodendro- |
| | cyte glycoprotein. |
| | (Q5STM2 321 aa linear PRI 13- |
| | SEP.-2005) |
| cffrdhsyqeeaamelkved | Myelin-oligodendrocyte glyco- |
| SEQ ID NO: 186 | protein Myelin oligodendro- |
| | cyte glycoprotein. |
| | (Q5STM2 321 aa linear PRI 13- |
| | SEP.-2005) |
| eeaamelkvedpfywvspgv | Myelin-oligodendrocyte glyco- |
| SEQ ID NO: 187 | protein Myelin oligodendro- |
| | cyte glycoprotein. |
| | (Q5STM2 321 aa linear PRI 13- |
| | SEP.-2005) |
| edpfywvspgvlvllavlpv | Myelin-oligodendrocyte glyco- |
| SEQ ID NO: 188 | protein Myelin oligodendro- |
| | cyte glycoprotein. |
| | (Q5STM2 321 aa linear PRI 13- |
| | SEP.-2005) |
| gvlvllavlpvlllqitvgl | Myelin-oligodendrocyte glyco- |
| SEQ ID NO: 189 | protein Myelin oligodendro- |
| | cyte glycoprotein. |
| | (Q5STM2 321 aa linear PRI 13- |
| | SEP.-2005) |
| pvlllqitvglvflclqyrl | Myelin-oligodendrocyte glyco- |
| SEQ ID NO: 190 | protein Myelin oligodendro- |
| | cyte glycoprotein. |
| | (Q5STM2 321 aa linear PRI 13- |
| | SEP.-2005) |
| glvflclqyrlrgklraeie | Myelin-oligodendrocyte glyco- |
| SEQ ID NO: 191 | protein Myelin oligodendro- |
| | cyte glycoprotein. |
| | (Q5STM2 321 aa linear PRI 13- |
| | SEP.-2005) |
| rlrgklraeienlhrtfdph | Myelin-oligodendrocyte glyco- |
| SEQ ID NO: 192 | protein Myelin oligodendro- |
| | cyte glycoprotein. |
| | (Q5STM2 321 aa linear PRI 13- |
| | SEP.-2005) |
| ienlhrtfdphflrvpcwki | Myelin-oligodendrocyte glyco- |
| SEQ ID NO: 193 | protein Myelin oligodendro- |
| | cyte glycoprotein. |
| | (Q5STM2 321 aa linear PRI 13- |
| | SEP.-2005) |
| phflrvpcwkitlfvivpvl | Myelin-oligodendrocyte glyco- |
| SEQ ID NO: 194 | protein Myelin oligodendro- |
| | cyte glycoprotein. |
| | (Q5STM2 321 aa linear PRI 13- |
| | SEP.-2005) |
| kitlfvivpvlgplvaliic | Myelin-oligodendrocyte glyco- |
| SEQ ID NO: 195 | protein Myelin oligodendro- |
| | cyte glycoprotein. |
| | (Q5STM2 321 aa linear PRI 13- |
| | SEP.-2005) |
| vlgplvaliicynwlhrrla | Myelin-oligodendrocyte glyco- |
| SEQ ID NO: 196 | protein Myelin oligodendro- |
| | cyte glycoprotein. |
| | (Q5STM2 321 aa linear PRI 13- |
| | SEP.-2005) |
| icynwlhrrlagqfleelps | Myelin-oligodendrocyte glyco- |
| SEQ ID NO: 197 | protein Myelin oligodendro- |
| | cyte glycoprotein. |
| | (Q5STM2 321 aa linear PRI 13- |
| | SEP.-2005) |
| lagqfleelpsridfpssvi | Myelin-oligodendrocyte glyco- |
| SEQ ID NO: 198 | protein Myelin oligodendro- |
| | cyte glycoprotein. |
| | (Q5STM2 321 aa linear PRI 13- |
| | SEP.-2005) |
| psridrpssviylfssfshf | Myelin-oligodendrocyte glyco- |
| SEQ ID NO: 199 | protein Myelin oligodendro- |
| | cyte glycoprotein. |
| | (Q5STM2 321 aa linear PRI 13- |
| | SEP.-2005) |
| viylfssfshfyypsfhfsl | Myelin-oligodendrocyte glyco- |
| SEQ ID NO: 200 | protein Myelin oligodendro- |
| | cyte glycoprotein. |
| | (Q5STM2 321 aa linear PRI 13- |
| | SEP.-2005) |
| hfyypslhfslqsstwkpsl | Myelin-oligodendrocyte glyco- |
| SEQ ID NO: 201 | protein Myelin oligodendro- |
| | cyte glycoprotein. |
| | (Q5STM2 321 aa linear PRI 13- |
| | SEP.-2005) |
| slqsstwkpslakdrqvpls | Myelin-oligodendrocyte glyco- |
| SEQ ID NO: 202 | protein Myelin oligodendro- |
| | cyte glycoprotein. |
| | (Q5STM2 321 aa linear PRI 13- |
| | SEP.-2005) |
| slakdrqvplspsedtctge | Myelin-oligodendrocyte glyco- |
| SEQ ID NO: 203 | protein Myelin oligodendro- |
| | cyte glycoprotein. |
| | (Q5STM2 321 aa linear PRI 13- |
| | SEP.-2005) |
| lspsedtctgeqhrmvsamn | Myelin-oligodendrocyte glyco- |
| SEQ ID NO: 204 | protein Myelin oligodendro- |
| | cyte glycoprotein. |
| | (Q5STM2 321 aa linear PRI 13- |
| | SEP.-2005) |
| geqhrmvsamnwrpgisslk | Myelin-oligodendrocyte glyco- |
| SEQ ID NO: 205 | protein Myelin oligodendro- |
| | cyte glycoprotein. |
| | (Q5STM2 321 aa linear PRI 13- |
| | SEP.-2005) |
| mgnhagkrelnaekastnse | myelin basic protein Myelin |
| SEQ ID NO: 206 | basic protein (MBP) (Myelin |
| | A1 protein) (Myelin membrane |
| | encephalitogenic protein) |
| | (P02686 304 aa linear PRI |
| lnaekastnsetnrgesekk | myelin basic protein Myelin |
| SEQ ID NO: 207 | basic protein (MBP) (Myelin |
| | A1 protein) (Myelin membrane |
| | encephalitogenic protein) |
| | (P02686 304 aa linear PRI |
| setnrgesekkrnlgelsrt | myelin basic protein Myelin |
| SEQ ID NO: 208 | basic protein (MBP) (Myelin |
| | A1 protein) (Myelin membrane |
| | encephalitogenic protein) |
| | (P02686 304 aa linear PRI |
| kkmlgelsrttsednevfg | myelin basic protein Myelin |
| SEQ ID NO: 209 | basic protein (MBP) (Myelin |
| | A1 protein) (Myelin membrane |
| | encephalitogenic protein) |
| | (P02686 304 aa linear PRI |
| rttsednevfgeadanqnng | myelin basic protein Myelin |
| SEQ ID N0: 210 | basic protein (MBP) (Myelin |
| | A1 protein) (Myelin membrane |
| | encephalitogenic protein) |
| | (P02686 304 aa linear PRI |
| fgeadanqnngtssqdtavt | myelin basic protein Myelin |
| SEQ ID NO: 211 | basic protein (MBP) (Myelin |
| | A1 protein) (Myelin membrane |
| | encephalitogenic protein) |
| | (P02686 304 aa linear PRI |
| ngtssqdtavtdskrtadpk | myelin basic protein Myelin |
| SEQ ID NO: 212 | basic protein (MBP) (Myelin |
| | A1 protein) (Myelin membrane |
| | encephalitogenic protein) |
| | (P02686 304 aa linear PRI |
| vtdskrtadpknawqdahpa | myelin basic protein Myelin |
| SEQ ID NO: 213 | basic protein (MBP) (Myelin |
| | A1 protein) (Myelin membrane |
| | encephalitogenic protein) |
| | (P02686 304 aa linear PRI |
| pknawqdahpadpgsrphli | myelin basic protein Myelin |
| SEQ ID NO: 214 | basic protein (MBP) (Myelin |
| | A1 protein) (Myelin membrane |
| | encephalitogenic protein) |
| | (P02686 304 aa linear PRI |
| padpgsrphlirlfsrdapg | myelin basic protein Myelin |
| SEQ ID NO: 215 | basic protein (MBP) (Myelin |
| | A1 protein) (Myelin membrane |
| | encephalitogenic protein) |
| | (P02686 304 aa linear PRI |
| lirlfsrdapgredntfkdr | myelin basic protein Myelin |
| SEQ ID NO: 216 | basic protein (MBP) (Myelin |
| | A1 protein) (Myelin membrane |
| | encephalitogenic protein) |
| | (P02686 304 aa linear PRI |
| pgredntfkdrpsesdelqt | myelin basic protein Myelin |
| SEQ ID NO: 217 | basic protein (MBP) (Myelin |
| | A1 protein) (Myelin membrane |
| | encephalitogenic protein) |
| | (P02686 304 aa linear PRI |
| drpsesdelqtiqedsasts | myelin basic protein Myelin |
| SEQ ID NO: 218 | basic protein (MBP) (Myelin |
| | A1 protein) (Myelin membrane |
| | encephalitogenic protein) |
| | (P02686 304 aa linear PRI |
| qtiqedsaatsesldvmasq | myelin basic protein Myelin |
| SEQ ID NO: 219 | basic protein (MBP) (Myelin |
| | A1 protein) (Myelin membrane |
| | encephalitogenic protein) |
| | (P02686 304 aa linear PRI |
| tsesldvmasqkrpsqrhgs | myelin basic protein Myelin |
| SEQ ID NO: 220 | basic protein (MBP) (Myelin |
| | A1 protein) (Myelin membrane |
| | encephalitogenic protein) |
| | (P02686 304 aa linear PRI |
| sqkrpsqrhgskylatastm | myelin basic protein Myelin |
| SEQ ID NO: 221 | basic protein (MBP) (Myelin |
| | A1 protein) (Myelin membrane |
| | encephalitogenic protein) |
| | (P02686 304 aa linear PRI |
| gskylatastmdharhgflp | myelin basic protein Myelin |
| SEQ ID NO: 222 | basic protein (MBP) (Myelin |
| | A1 protein) (Myelin membrane |
| | encephalitogenic protein) |
| | (P02686 304 aa linear PRI |
| tmdharhgflprhrdtgild | myelin basic protein Myelin |
| SEQ ID NO: 223 | basic protein (MBP) (Myelin |
| | A1 protein) (Myelin membrane |
| | encephalitogenic protein) |
| | (P02686 304 aa linear PRI |
| lprhrdtgildsigrffggd | myelin basic protein Myelin |
| SEQ ID NO: 224 | basic protein (MBP) (Myelin |
| | A1 protein) (Myelin membrane |
| | encephalitogenic protein) |
| | (P02686 304 aa linear PRI |
| ldsigrffggdrgapkrgsg | myelin basic protein Myelin |
| SEQ ID NO: 225 | basic protein (MBP) (Myelin |
| | A1 protein) (Myelin membrane |
| | encephalitogenic protein) |
| | (P02686 304 aa linear PRI |
| gdrgapkrgsgkdshhpart | myelin basic protein Myelin |
| SEQ ID NO: 226 | basic protein (MBP) (Myelin |
| | A1 protein) (Myelin membrane |
| | encephalitogenic protein) |
| | (P02686 304 aa linear PRI |
| sgkdshhpartahygslpqk | myelin basic protein Myelin |
| SEQ ID NO: 227 | basic protein (MBP) (Myelin |
| | A1 protein) (Myelin membrane |
| | encephalitogenic protein) |
| | (P02686 304 aa linear PRI |
| rtahygslpqkshgrtqden | myelin basic protein Myelin |
| SEQ ID NO: 228 | basic protein (MBP) (Myelin |
| | A1 protein) (Myelin membrane |
| | encephalitogenic protein) |
| | (P02686 304 aa linear PRI |
| qkshgrtqdenpvvhffkni | myelin basic protein Myelin |
| SEQ ID NO: 229 | basic protein (MBP) (Myelin |
| | A1 protein) (Myelin membrane |
| | encephalitogenic protein) |
| | (P02686 304 aa linear PRI |
| enpvvhffknivtprtppps | myelin basic protein Myelin |
| SEQ ID NO: 230 | basic protein (MBP) (Myelin |
| | A1 protein) (Myelin membrane |
| | encephalitogenic protein) |
| | (P02686 304 aa linear PRI |
| nivtprtpppsqgkgrglsl | myelin basic protein Myelin |
| SEQ ID NO: 231 | basic protein (MBP) (Myelin |
| | A1 protein) (Myelin membrane |
| | encephalitogenic protein) |
| | (P02686 304 aa linear PRI |
| psqgkgrglslsrfswgaeg | myelin basic protein Myelin |
| SEQ ID NO: 232 | basic protein (MBP) (Myelin |
| | A1 protein) (Myelin membrane |
| | encephalitogenic protein) |
| | (P02686 304 aa linear PRI |
| slsrfswgaegqrpgfgygg | myelin basic protein Myelin |
| SEQ ID NO: 233 | basic protein (MBP) (Myelin |
| | A1 protein) (Myelin membrane |
| | encephalitogenic protein) |
| | (P02686 304 aa linear PRI |
| egqrpgfgyggrasdyksah | myelin basic protein Myelin |
| SEQ ID NO: 234 | basic protein (MBP) (Myelin |
| | A1 protein) (Myelin membrane |
| | encephalitogenic protein) |
| | (P02686 304 aa linear PRI |
| ggrasdyksahkgfkgvdaq | myelin basic protein Myelin |
| SEQ ID NO: 235 | basic protein (MBP) (Myelin |
| | A1 protein) (Myelin membrane |
| | encephalitogenic protein) |
| | (P02686 304 aa linear PRI |
| ahkgfkgvdaqgtlskifkl | myelin basic protein Myelin |
| SEQ ID NO: 236 | basic protein (MBP) (Myelin |
| | A1 protein) (Myelin membrane |
| | encephalitogenic protein) |
| | (P02686 304 aa linear PRI |
| aqgtlskifklggrdsrsgs | myelin basic protein Myelin |
| SEQ ID NO: 237 | basic protein (MBP) (Myelin |
| | A1 protein) (Myelin membrane |
| | encephalitogenic protein) |
| | (P02686 304 aa linear PRI |
| raeienlhrtfesfgvlgpq | Myelin-oligodendrocyte |
| SEQ ID NO: 238 | glycoprotein Myelin oligo- |
| | dendrocyte glycoprotein. |
| | (Q5STL9 298 aa linear PRI 13- |
| | SEP.-2005) |
| tfesfgvlgpqvkepkktgq | Myelin-oligodendrocyte |
| SEQ ID NO: 239 | glycoprotein Myelin oligo- |
| | dendrocyte glycoprotein. |
| | (Q5STL9 298 aa linear PRI 13- |
| | SEP.-2005) |
| pqvkcpkktgqfleelpsri | Myelin-oligodendrocyte |
| SEQ ID NO: 240 | glycoprotein Myelin oligo- |
| | dendrocyte glycoprotein. |
| | (Q5STL9 298 aa linear PRI 13- |
| | SEP.-2005) |
| rrlagqfleelrkfsslcyk | Myelin-oligodendrocyte |
| SEQ ID NO: 241 | glycoprotein MOG protein |
| | (Myelin oligodendrocyte |
| | glycoprotein). |
| | (Q81YG5 295 aa linear PRI 13- |
| | SEP.-200) |
| elrkfsslcykqriksqere | Myelin-oligodendrocyte |
| SEQ ID NO: 242 | glycoprotein MOG protein |
| | (Myelin oligodendrocyte |
| | glycoprotein). |
| | (Q81YG5 295 aa linear PRI 13- |
| | SEP.-200) |
| ykqriksqereteatrgrgg | Myelin-oligodendrocyte |
| SEQ ID NO: 243 | glycoprotein MOG protein |
| | (Myelin oligodendrocyte |
| | glycoprotein). |
| | (Q81YG5 295 aa linear PRI 13- |
| | SEP.-200) |
| reteatrgrggllrdhiprg | Myelin-oligodendrocyte |
| SEQ ID NO: 244 | glycoprotein MOG protein |
| | (Myelin oligodendrocyte |
| | glycoprotein). |
| | (Q81YG5 295 aa linear PRI 13- |
| | SEP.-200) |
| ggllrdhiprgkeeleslgg | Myelin-oligodendrocyte |
| SEQ ID NO: 245 | glycoprotein MOG protein |
| | (Myelin oligodendrocyte |
| | glycoprotein). |
| | (Q81YG5 295 aa linear PRI 13- |
| | SEP.-200) |
| raeienlhrtfgqfleelps | Myelin-oligodendrocyte glyco- |
| SEQ ID NO: 246 | protein Myelin oligodendro- |
| | cyte glycoprotein. |
| | (Q5STM0 282 aa linear PRI 13- |
| | SEP.-2005) |
| mglleccarclvgapfaslv | proteolipid protein Proteo- |
| SEQ ID NO: 247 | lipid protein 1, isoform 1. |
| | (Q502Y1 277 aa linear PRI 13- |
| | SEP.-2005) |
| clvgapfaslvatglcffgv | proteolipid protein Proteo- |
| SEQ ID NO: 248 | lipid protein 1, isoform 1. |
| | (Q502Y1 277 aa linear PRI 13- |
| | SEP.-2005) |
| lvatglcffgvalfcgcghe | proteolipid protein Proteo- |
| SEQ ID NO: 249 | lipid protein 1, isoform 1. |
| | (Q502Y1 277 aa linear PRI 13- |
| | SEP.-2005) |
| gvalfcgcghealtgtekli | proteolipid protein Proteo- |
| SEQ ID NO: 250 | lipid protein 1, isoform 1. |
| | (Q502Y1 277 aa linear PRI 13- |
| | SEP.-2005) |
| healtgteklietyfsknyq | proteolipid protein Proteo- |
| SEQ ID NO: 251 | lipid protein 1, isoform 1. |
| | (Q502Y1 277 aa linear PRI 13- |
| | SEP.-2005) |
| lietyfsknyqdyeylinvi | proteolipid protein Proteo- |
| SEQ ID NO: 252 | lipid protein 1, isoform 1. |
| | (Q502Y1 277 aa linear PRI 13- |
| | SEP.-2005) |
| yqdyeylinvihafqyviyg | proteolipid protein Proteo- |
| SEQ ID NO: 253 | lipid protein 1, isoform 1. |
| | (Q502Y1 277 aa linear PRI 13- |
| | SEP.-2005) |
| vihafqyviygtasffflyg | proteolipid protein Proteo- |
| SEQ ID NO: 254 | lipid protein 1, isoform 1. |
| | (Q502Y1 277 aa linear PRI 13- |
| | SEP.-2005) |
| ygtasffflygalllaegfy | proteolipid protein Proteo- |
| SEQ ID NO: 255 | lipid protein 1, isoform 1. |
| | (Q502Y1 277 aa linear PRI 13- |
| | SEP.-2005) |
| ygalllaegfyttgavrqif | proteolipid protein Proteo- |
| SEQ ID NO: 256 | lipid protein 1, isoform 1. |
| | (Q502Y1 277 aa linear PRI 13- |
| | SEP.-2005) |
| fyttgavrqifgdyktticg | proteolipid protein Proteo- |
| SEQ ID NO: 257 | lipid protein 1, isoform 1. |
| | (Q502Y1 277 aa linear PRI 13- |
| | SEP.-2005) |
| ifgdyktticgkglsatvtg | proteolipid protein Proteo- |
| SEQ ID NO: 258 | lipid protein 1, isoform 1. |
| | (Q502Y1 277 aa linear PRI 13- |
| | SEP.-2005) |
| cgkglsatvtggqkgrgsrg | proteolipid protein Proteo- |
| SEQ ID NO: 259 | lipid protein 1, isoform 1. |
| | (Q502Y1 277 aa linear PRI 13- |
| | SEP.-2005) |
| tggqkgrgsrgqhqahsler | proteolipid protein Proteo- |
| SEQ ID NO: 260 | lipid protein 1, isoform 1. |
| | (Q502Y1 277 aa linear PRI 13- |
| | SEP.-2005) |
| rgqhqahslervchclgkwl | proteolipid protein Proteo- |
| SEQ ID NO: 261 | lipid protein 1, isoform 1. |
| | (Q502Y1 277 aa linear PRI 13- |
| | SEP.-2005) |
| ervehclgkwlghpdkfvgi | proteolipid protein Proteo- |
| SEQ ID NO: 262 | lipid protein 1, isoform 1. |
| | (Q502Y1 277 aa linear PRI 13- |
| | SEP.-2005) |
| wlghpdkfvgityaltvvwl | proteolipid protein Proteo- |
| SEQ ID NO: 263 | lipid protein 1, isoform 1. |
| | (Q502Y1 277 aa linear PRI 13- |
| | SEP.-2005) |
| gityaltvvwllvfacsavp | proteolipid protein Proteo- |
| SEQ ID NO: 264 | lipid protein 1, isoform 1. |
| | (Q502Y1 277 aa linear PRI 13- |
| | SEP.-2005) |
| wllvfacsavpvyiyfntwt | proteolipid protein Proteo- |
| SEQ ID NO: 265 | lipid protein 1, isoform 1. |
| | (Q502Y1 277 aa linear PRI 13- |
| | SEP.-2005) |
| vpvyiyfntwttcqsiafps | proteolipid protein Proteo- |
| SEQ ID NO: 266 | lipid protein 1, isoform 1. |
| | (Q502Y1 277 aa linear PRI 13- |
| | SEP.-2005) |
| wttcqsiafpsktsasigsl | proteolipid protein Proteo- |
| SEQ ID NO: 267 | lipid protein 1, isoform 1. |
| | (Q502Y1 277 aa linear PRI 13- |
| | SEP.-2005) |
| psktsasigslcadarmygv | proteolipid protein Proteo- |
| SEQ ID NO: 268 | lipid protein 1, isoform 1. |
| | (Q502Y1 277 aa linear PRI 13- |
| | SEP.-2005) |
| slcadarmygvlpwnafpgk | proteolipid protein Proteo- |
| SEQ ID NO: 269 | lipid protein 1, isoform 1. |
| | (Q502Y1 277 aa linear PRI 13- |
| | SEP.-2005) |
| gvlpwnafpgkvcgsnllsi | proteolipid protein Proteo- |
| SEQ ID NO: 270 | lipid protein 1, isoform 1. |
| | (Q502Y1 277 aa linear PRI 13- |
| | SEP.-2005) |
| gkvcgsnllsicktaefqmt | proteolipid protein Proteo- |
| SEQ ID NO: 271 | lipid protein 1, isoform 1. |
| | (Q502Y1 277 aa linear PRI 13- |
| | SEP.-2005) |
| sicktaefqmtfhlfiaafv | proteolipid protein Proteo- |
| SEQ ID NO: 272 | lipid protein 1, isoform 1. |
| | (Q502Y1 277 aa linear PRI 13- |
| | SEP.-2005) |
| mtfhlfiaafvgaaatlvsl | proteolipid protein Proteo- |
| SEQ ID NO: 273 | lipid protein 1, isoform 1. |
| | (Q502Y1 277 aa linear PRI 13- |
| | SEP.-2005) |
| fvgaaatlvslltfmiaaty | proteolipid protein Proteo- |
| SEQ ID NO: 274 | lipid protein 1, isoform 1. |
| | (Q502Y1 277 aa linear PRI 13- |
| | SEP.-2005) |
| slltfmiaatynfavlklmg | proteolipid protein Proteo- |
| SEQ ID NO: 275 | lipid protein 1, isoform 1. |
| | (Q502Y1 277 aa linear PRI 13- |
| | SEP.-2005) |
| vyiyfntwttcdsiafpskt | proteolipid protein Proteo- |
| SEQ ID NO: 276 | lipid protein |
| | (AAA59565 277 aa linear PRI |
| | 07-JAN.-199) |
| adarmygvlpwiafpgkvcg | proteolipid protein Proteo- |
| SEQ ID NO: 277 | lipid protein |
| | (AAA59565 277 aa linear PRI |
| | 07-JAN.-199) |
| qhqahslervctclgkwlgh | proteolipid protein Proteo- |
| SEQ ID NO: 278 | lipid protein |
| | (AAA59565 277 aa linear PRI |
| | 07-JAN.-199) |
| pccggslspgtvemaslsrp | Myelin-oligodendrocyte glyco- |
| SEQ ID NO: 279 | protein Myelin oligodendro- |
| | cyte glycoprotein isoform |
| | alpha1 variant. |
| | (Q59EJ6 259 aa linear PRI 10- |
| | MAY-2005) |
| pvlllqitvgliflclqyrl | Myelin-oligodendrocyte glyco- |
| SEQ ID NO: 280 | protein Myelin oligodendro- |
| | cyte glycoprotein. |
| | (Q5SUK7 252 aa linear PRI 01- |
| | FEB.-2005) |
| rrlagqfleellfhlealsg | Myelin-oligodendrocyte glyco- |
| SEQ ID NO: 281 | protein Myelin oligodendro- |
| | cyte glycoprotein. |
| | (Q5SUK7 252 aa linear PRI 01- |
| | FEB.-2005) |
| avlpvlllqitlglvflclq | Myelin-oligodcndrocyte glyco- |
| SEQ ID NO: 282 | protein |
| | (CAA88109 247 aa linear PRI |
| | 21-AUG.-1996) |
| tticgkglsatfvgityalt | proteolipid protein PLP1 |
| SEQ ID NO: 283 | protein. |
| | (Q6FHZ6 242 aa linear PRI 10- |
| | MAY-200) |
| apfaslvatglgffgvalfc | proteolipid protein Proteo- |
| SEQ ID NO: 284 | lipid protein I (Pelizaeus- |
| | Merzbacher disease, spastic |
| | paraplegia 2, uncomplicated). |
| | (Q5U0F3 242 aa linear PRI |
| tfmiaatynfailklmgrgt | proteolipid protein Proteo- |
| SEQ ID NO: 285 | lipid protein I (Pelizaeus- |
| | Merzbacher disease, spastic |
| | paraplegia 2, uncomplicated). |
| | (Q5U0F3 242 aa linear PRI |
| mvatclqvvgfvtsfvgwig | oligodendrocyte-specific |
| SEQ ID NO: 286 | protein |
| | (AAC25187 218 aa linear PR1 |
| | 02-JUL.-1998) |
| gfvtsfvgwigvivttstnd | oligodendrocyte-specific |
| SEQ ID NO: 287 | protein |
| | (AAC25187 218 aa linear PR1 |
| | 02-JUL.-1998) |
| igvivttstndwvvtcgyti | oligodendrocyte-specific |
| SEQ ID NO: 288 | protein |
| | (AAC25187 218 aa linear PR1 |
| | 02-JUL.-1998) |
| ndwvvtcgytiptcrkldel | oligodendrocyte-specific |
| SEQ ID NO: 289 | protein |
| | (AAC25187 218 aa linear PR1 |
| | 02-JUL.-1998) |
| tiptcrkldelgskglwadc | oligodendrocyte-specific |
| SEQ ID NO: 290 | protein |
| | (AAC25187 218 aa linear PR1 |
| | 02-JUL.-1998) |
| elgskglwadcvmatglyhc | oligodendrocyte-specific |
| SEQ ID NO: 291 | protein |
| | (AAC25187 218 aa linear PR1 |
| | 02-JUL.-1998) |
| dcvmatglyhckplvdilil | oligodendrocyte-specific |
| SEQ ID NO: 292 | protein |
| | (AAC25187 218 aa linear PR1 |
| | 02-JUL.-1998) |
| hckplvdililpgyvqacra | oligodendrocyte-specific |
| SEQ ID NO: 293 | protein |
| | (AAC25187 218 aa linear PR1 |
| | 02-JUL.-1998) |
| ilpgyvqacralmiaasvlg | oligodendrocyte-specific |
| SEQ ID NO: 294 | protein |
| | (AAC25187 218 aa linear PR1 |
| | 02-JUL.-1998) |
| ralmiaasvlglpaillllt | oligodendrocyte-specific |
| SEQ ID NO: 295 | protein |
| | (AAC25187 218 aa linear PR1 |
| | 02-JUL.-1998) |
| lglpailllltvlpcirmgq | oligodendrocyte-specific |
| SEQ ID NO: 296 | protein |
| | (AAC25187 218 aa linear PR1 |
| | 02-JUL.-1998) |
| ltvlpcirmgqepgvakyrr | oligodendrocyte-specific |
| SEQ ID NO: 297 | protein |
| | (AAC25187 218 aa linear PR1 |
| | 02-JUL.-1998) |
| gqepgvakyrraqlagvlli | oligodendrocyte-specific |
| SEQ ID NO: 298 | protein |
| | (AAC25187 218 aa linear PR1 |
| | 02-JUL.-1998) |
| rraqlagvllillalcalva | oligodendrocyte-specific |
| SEQ ID NO: 299 | protein |
| | (AAC25187 218 aa linear PR1 |
| | 02-JUL.-1998) |
| lillalcalvatiwfpvcah | oligodendrocyte-specific |
| SEQ ID NO: 300 | protein |
| | (AAC25187 218 aa linear PR1 |
| | 02-JUL.-1998) |
| vatiwfpvcahrettivsfg | oligodendrocyte-specific |
| SEQ ID NO: 301 | protein |
| | (AAC25187 218 aa linear PR1 |
| | 02-JUL.-1998) |
| ahrettivsfgyslyagwig | oligodendrocyte-specific |
| SEQ ID NO: 302 | protein |
| | (AAC25187 218 aa linear PR1 |
| | 02-JUL.-1998) |
| fgyslyagwigavlclvggc | oligodendrocyte-specific |
| SEQ ID NO: 303 | protein |
| | (AAC25187 218 aa linear PR1 |
| | 02-JUL.-1998) |
| igavlclvggcvilccagda | oligodendrocyte-specific |
| SEQ ID NO: 304 | protein |
| | (AAC25187 218 aa linear PR1 |
| | 02-JUL.-1998) |
| gcvilccagdaqafgenvst | oligodendrocyte-specific |
| SEQ ID NO: 305 | protein |
| | (AAC25187 218 aa linear PR1 |
| | 02-JUL.-1998) |
| daqafgenvsttlralaprl | oligodendrocyte-specific |
| SEQ ID NO: 306 | protein |
| | (AAC25187 218 aa linear PR1 |
| | 02-JUL.-1998) |
| sttlralaprlmrrvptykr | oligodendrocyte-specific |
| SEQ ID NO: 307 | protein |
| | (AAC25187 218 aa linear PR1 |
| | 02-JUL.-1998) |
| rlmrrvptykraarlptevl | oligodendrocyte-specific |
| SEQ ID NO: 308 | protein |
| | (AAC25187 218 aa linear PR1 |
| | 02-JUL.-1998) |
| raeienlhrtfvfhlealsg | Myelin-oligodendrocyte glyco- |
| SEQ ID NO: 309 | protein Myelin oligodendro- |
| | cyte gycoprotein. |
| | (Q5SUK4 206 aa linear PRI 01- |
| | FEB.-2005) |
| llllqvsssyadpfywvspg | Myelin-oligodendrocyte glyco- |
| SEQ ID NO: 310 | protein Myelin oligodendro- |
| | cyte glycoprotein. |
| | (Q5STM1 205 aa linear PRI 13- |
| | SEP.-2005) |
| drgapkrgsgkvpwlkpgrs | myelin basic protein Myelin |
| SEQ ID NO: 311 | basic protein. |
| | (Q65ZS4 203 aa linear PRI 25- |
| | OCT.-2004) |
| gkvpwlkpgrsplpsharsq | myelin basic protein Myelin |
| SEQ ID NO: 312 | basic protein. |
| | (Q65ZS4 203 aa linear PRI 25- |
| | OCT.-2004) |
| rsplpsharsqpglcnmykd | myelin basic protein Myelin |
| SEQ ID NO: 313 | basic protein. |
| | (Q65ZS4 203 aa linear PRI 25- |
| | OCT.-2004) |
| sqpglcnmykdshhpartah | myelin basic protein Myelin |
| SEQ ID NO: 314 | basic protein. |
| | (Q65ZS4 203 aa linear PRI 25- |
| | OCT.-2004) |
| klggrdsrsgspmarrhhhh | myelin basic protein Myelin |
| SEQ ID NO: 315 | basic protein. |
| | (Q65ZS4 203 aa linear PRI 25- |
| | OCT.-2004) |
| mdiaihhpwirrpffpthsp | crystallin unknown [Homo |
| SEQ ID NO: 316 | sapiens]. |
| | (AAC19161 194 aa linear PRI |
| | 08-JUN.-1998) |
| irrpffpfhspsrlfdqffg | crystallin unknown [Homo |
| SEQ ID NO: 317 | sapiens]. |
| | (AAC19161 194 aa linear PRI |
| | 08-JUN.-1998) |
| spsrlfdqffgehllesdlf | crystallin unknown [Homo |
| SEQ ID NO: 318 | sapiens]. |
| | (AAC19161 194 aa linear PRI |
| | 08-JUN.-1998) |
| fgehllesdlfptstslspf | crystallin unknown [Homo |
| SEQ ID NO: 319 | sapiens]. |
| | (AAC19161 194 aa linear PRI |
| | 08-JUN.-1998) |
| lfptstslspfylrppsflr | crystallin unknown [Homo |
| SEQ ID NO: 320 | sapiens]. |
| | (AAC19161 194 aa linear PRI |
| | 08-JUN.-1998) |
| pfylrppsflrapswfdtgl | crystallin unknown [Homo |
| SEQ ID NO: 321 | sapiens]. |
| | (AAC19161 194 aa linear PRI |
| | 08-JUN.-1998) |
| lrapswfdtglsemrlekdr | crystallin unknown [Homo |
| SEQ ID NO: 322 | sapiens]. |
| | (AAC19161 194 aa linear PRI |
| | 08-JUN.-1998) |
| glsemrlekdrfsvnldvkh | crystallin unknown [Homo |
| SEQ ID NO: 323 | sapiens]. |
| | (AAC19161 194 aa linear PRI |
| | 08-JUN.-1998) |
| drfsvnldvkhfspeelkvk | crystallin unknown [Homo |
| SEQ ID NO: 324 | sapiens]. |
| | (AAC19161 194 aa linear PRI |
| | 08-JUN.-1998) |
| khfspeelkvkvlgdvievh | crystallin unknown [Homo |
| SEQ ID NO: 325 | sapiens]. |
| | (AAC19161 194 aa linear PRI |
| | 08-JUN.-1998) |
| vkvlgdvievhgkheerqde | crystallin unknown [Homo |
| SEQ ID NO: 326 | sapiens]. |
| | (AAC19161 194 aa linear PRI |
| | 08-JUN.-1998) |
| vhgkheerqdehgfisrefh | crystallin unknown [Homo |
| SEQ ID NO: 327 | sapiens]. |
| | (AAC19161 194 aa linear PRI |
| | 08-JUN.-1998) |
| dehgfisrefhrkyripadv | crystallin unknown [Homo |
| SEQ ID NO: 328 | sapiens]. |
| | (AAC19161 194 aa linear PRI |
| | 08-JUN.-1998) |
| fhrkyripadvdpltitssl | crystallin unknown [Homo |
| SEQ ID NO: 329 | sapiens]. |
| | (AAC19161 194 aa linear PRI |
| | 08-JUN.-1998) |
| dvdpltitsslssdgvltvn | crystallin unknown [Homo |
| SEQ ID NO: 330 | sapiens]. |
| | (AAC19161 194 aa linear PRI |
| | 08-JUN.-1998) |
| slssdgvltvngprkqvsgp | crystallin unknown [Homo |
| SEQ ID NO: 331 | sapiens]. |
| | (AAC19161 194 aa linear PRI |
| | 08-JUN.-1998) |
| vngprkqvsgpertipitre | crystallin unknown [Homo |
| SEQ ID NO: 332 | sapiens]. |
| | (AAC19161 194 aa linear PRI |
| | 08-JUN.-1998) |
| gpertipitrekkpavtaap | crystallin unknown [Homo |
| SEQ ID NO: 333 | sapiens]. |
| | (AAC19161 194 aa linear PRI |
| | 08-JUN.-1998) |
| rekkpavtnapkkkmpflel | crystallin unknown [Homo |
| SEQ ID NO: 334 | sapiens]. |
| | (AAC19161 194 aa linear PRI |
| | 08-JUN.-1998) |
| apkkkmpflelhflkqesfp | crystallin unknown [Homo |
| SEQ ID NO: 335 | sapiens]. |
| | (AAC19161 194 aa linear PRI |
| | 08-JUN.-1998) |
| prtpppsqgkgaegqrpgfg | myelin basic protein myelin |
| SEQ ID NO: 336 | basic protein. |
| | (AAA59563 186 aa linear PRI |
| | 07-JAN.-199) |
| gpertipitreekpavtaap | crystallin Alpha crystallin B |
| SEQ ID NO: 337 | chain (Alpha(B)-crystallin) |
| | (Rosenthal fiber component) |
| | (Heat-shock protein beta-5) |
| | (HspB5). |
| | (P02511 175 aa |
| qeeaamclkvevshsvtqdw | Myelin-oligodendrocyte glyco- |
| SEQ ID NO: 338 | protein isoform beta4 |
| | precursor |
| | (NP_996536 171 aa) |
| vevshsvtqdwlqwhdhgst | Myelin-oligodendrocyte glyco- |
| SEQ ID NO: 339 | protein isoform beta4 |
| | precursor |
| | (NP_996536 171 aa) |
| gildsigrffgsdraapkrg | myelin basic protein |
| SEQ ID NO: 340 | (P25188 167 aa linear ROD |
| | 24-JAN.-2006 ACCESSION |
| | P25188 VERSION P25188 GI: |
| | 12679) |
| pkrgsgkdshhaartthygs | myelin basic protein |
| SEQ ID NO: 341 | (P25188 167 aa linear ROD |
| | 24-JAN.-2006 ACCESSION |
| | P25188 VERSION P25188 GI: |
| | 12679) |
| hhaartthygslpqksqrsq | myelin basic protein |
| SEQ ID NO: 342 | (P25188 167 aa linear ROD |
| | 24-JAN.-2006 ACCESSION |
| | P25188 VERSION P25188 GI: |
| | 12679) |
| gslpqksqrsqdenpvvhff | myelin basic protein |
| SEQ ID NO: 343 | (P25188 167 aa linear ROD |
| | 24-JAN.-2006 ACCESSION |
| | P25188 VERSION P25188 GI: |
| | 12679) |
| lsrfswgaegqkpgfgyggr | myelin basic protein |
| SEQ ID NO: 344 | (P25188 167 aa linear ROD |
| | 24-JAN.-2006 ACCESSION |
| | P25188 VERSION P25188 GI: |
| | 12679) |
| gqkpgfgyggradykskgfk | myelin basic protein |
| SEQ ID NO: 345 | (P25188 167 aa linear ROD |
| | 24-JAN.-2006 ACCESSION |
| | P25188 VERSION P25188 GI: |
| | 12679) |
| gradykskgfkgahdaqgtl | myelin basic protein |
| SEQ ID NO: 346 | (P25188 167 aa linear ROD |
| | 24-JAN.-2006 ACCESSION |
| | P25188 VERSION P25188 GI: |
| | 12679) |
| mhgfktpssqslqtklsvhl | myelin basic protein Hypo- |
| SEQ ID NO: 347 | thetical protein FLJ44910 |
| | (Q6ZT70 154 aa linear PRI 05- |
| | JUL.-2004) |
| qslqtklsvhliclvwqmrw | myelin basic protein Hypo- |
| SEQ ID NO: 348 | thetical protein FLJ44910 |
| | (Q6ZT70 154 aa linear PRI 05- |
| | JUL.-2004) |
| hliclvwqmrwhiggtspgm | myelin basic protein Hypo- |
| SEQ ID NO: 349 | thetical protein FLJ44910 |
| | (Q6ZT70 154 aa linear PRI 05- |
| | JUL.-2004) |
| rwhiggtspgmdtwlcslla | myelin basic protein Hypo- |
| SEQ ID NO: 350 | thetical protein FLJ44910 |
| | (Q6ZT70 154 aa linear PRI 05- |
| | JUL.-2004) |
| gmdtwlcsllarvargvlgs | myelin basic protein Hypo- |
| SEQ ID NO: 351 | thetical protein FLJ44910 |
| | (Q6ZT70 154 aa linear PRI 05- |
| | JUL.-2004) |
| larvargvlgsrrkrscscc | myelin basic protein Hypo- |
| SEQ ID NO: 352 | thetical protein FLJ44910 |
| | (Q6ZT70 154 aa linear PRI 05- |
| | JUL.-2004) |
| gsrrkrscsccrwqfpsaal | myelin basic protein Hypo- |
| SEQ ID NO: 353 | thetical protein FLJ44910 |
| | (Q6ZT70 154 aa linear PRI 05- |
| | JUL.-2004) |
| ccrwqfpsaalapladekkc | myelin basic protein Hypo- |
| SEQ ID NO: 354 | thetical protein FLJ44910 |
| | (Q6ZT70 154 aa linear PRI 05- |
| | JUL.-2004) |
| alapladekkcsfkakqwsg | myelin basic protein Hypo- |
| SEQ ID NO: 355 | thetical protein FLJ44910 |
| | (Q6ZT70 154 aa linear PRI 05- |
| | JUL.-2004) |
| kcsfkakqwsgctfthrsaa | myelin basic protein Hypo- |
| SEQ ID NO: 356 | thetical protein FLJ44910 |
| | (Q6ZT70 154 aa linear PRI 05- |
| | JUL.-2004) |
| sgctfthrsaalfcravvmp | myelin basic protein Hypo- |
| SEQ ID NO: 357 | thetical protein FLJ44910 |
| | (Q6ZT70 154 aa linear PRI 05- |
| | JUL.-2004) |
| aalfcravvmpgttpwglgi | myelin basic protein Hypo- |
| SEQ ID NO: 358 | thetical protein FLJ44910 |
| | (Q6ZT70 154 aa linear PRI 05- |
| | JUL.-2004) |
| mpgttpwglgirfqfsltel | myelin basic protein Hypo- |
| SEQ ID NO: 359 | thetical protein FLJ44910 |
| | (Q6ZT70 154 aa linear PRI 05- |
| | JUL.-2004) |
| girfqfslteleiqlafaeg | myelin basic protein Hypo- |
| SEQ ID NO: 360 | thetical protein FLJ44910 |
| | (Q6ZT70 154 aa linear PRI 05- |
| | JUL.-2004) |
| eleiqlafaeggrekwnekt | myelin basic protein Hypo- |
| SEQ ID NO: 361 | thetical protein FLJ44910 |
| | (Q6ZT70 154 aa linear PRI 05- |
| | JUL.-2004) |
| mcgwaaylmrepppkpepss | myelin basic protein Hypo- |
| SEQ ID NO: 362 | thetical protein FLJ42840 |
| | (Hypothetical protein |
| | FLJ41439). |
| | (Q6ZVA1 144 aa linear PRI 01- |
| | FEB.-2005) |
| repppkpepsslcgqhpgrp | myelin basic protein Hypo- |
| SEQ ID NO: 363 | thetical protein FLJ42840 |
| | (Hypothetical protein |
| | FLJ41439). |
| | (Q6ZVA1 144 aa linear PRI 01- |
| | FEB.-2005) |
| sslcgqhpgrpggaaspapt | myelin basic protein Hypo- |
| SEQ ID NO: 364 | thetical protein FLJ42840 |
| | (Hypothetical protein |
| | FLJ41439). |
| | (Q6ZVA1 144 aa linear PRI 01- |
| | FEB.-2005) |
| rpggaaspaptpsawcwpva | myelin basic protein Hypo- |
| SEQ ID NO: 365 | thetical protein FLJ42840 |
| | (Hypothetical protein |
| | FLJ41439). |
| | (Q6ZVA1 144 aa linear PRI 01- |
| | FEB.-2005) |
| ptpsawcwpvapepltspvr | myelin basic protein Hypo- |
| SEQ ID NO: 366 | thetical protein FLJ42840 |
| | (Hypothetical protein |
| | FLJ41439). |
| | (Q6ZVA1 144 aa linear PRI 01- |
| | FEB.-2005) |
| vapepltspvrglgpspgpw | myelin basic protein Hypo- |
| SEQ ID NO: 367 | thetical protein FLJ42840 |
| | (Hypothetical protein |
| | FLJ41439). |
| | (Q6ZVA1 144 aa linear PRI 01- |
| | FEB.-2005) |
| vrglgpspgpwwqlpvaqaa | myelin basic protein Hypo- |
| SEQ ID NO: 368 | thetical protein FLJ42840 |
| | (Hypothetical protein |
| | FLJ41439). |
| | (Q6ZVA1 144 aa linear PRI 01- |
| | FEB.-2005) |
| pwwqlpvaqaacpaprveve | myelin basic protein Hypo- |
| SEQ ID NO: 369 | thetical protein FLJ42840 |
| | (Hypothetical protein |
| | FLJ41439). |
| | (Q6ZVA1 144 aa linear PRI 01- |
| | FEB.-2005) |
| aacpaprvevelrglllqga | myelin basic protein Hypo- |
| SEQ ID NO: 370 | thetical protein FLJ42840 |
| | (Hypothetical protein |
| | FLJ41439). |
| | (Q6ZVA1 144 aa linear PRI 01- |
| | FEB.-2005) |
| velrglllqgaegqrpgfgy | myelin basic protein Hypo- |
| SEQ ID NO: 371 | thetical protein FLJ42840 |
| | (Hypothetical protein |
| | FLJ41439). |
| | (Q6ZVA1 144 aa linear PRI 01- |
| | FEB.-2005) |
| grglslsrfswreggrrpet | myelin basic protein iso 5 |
| SEQ ID NO: 372 | (NP_001020265 137 aa linear |
| | PRI 18-OCT.-200) |
| swreggrrpetriwlrrqsv | myelin basic protein iso 5 |
| SEQ ID NO: 373 | (NP_001020265 137 aa linear |
| | PRI 18-OCT.-200) |
| tprtpppsqgkreggrrpet | myelin basic protein iso 6 |
| SEQ ID NO: 374 | (NP_001020269 126 aa linear |
| | PRI 18-OCT.-200) |
| mselekamvalidvfhqysg | S100 S-100 calcium-binding |
| SEQ ID NO: 375 | protein beta subunit (S-100 |
| | protein, beta chain). |
| | (P04271 92 aa linear PRI 24- |
| | JAN.-2006) |
| alidvfhqysgregdkhklk | S100 S-100 calcium-binding |
| SEQ ID NO: 376 | protein beta subunit (S-100 |
| | protein, beta chain). |
| | (P04271 92 aa linear PRI 24- |
| | JAN.-2006) |
| sgregdkhklkkselkelin | S100 S-100 calcium-binding |
| SEQ ID NO: 377 | protein beta subunit (S-100 |
| | protein, beta chain). |
| | (P04271 92 aa linear PRI 24- |
| | JAN.-2006) |
| lkkselkelinnelshflee | S100 S-100 calcium-binding |
| SEQ ID NO: 378 | protein beta subunit (S-100 |
| | protein, beta chain). |
| | (P04271 92 aa linear PRI 24- |
| | JAN.-2006) |
| innelshfleeikeqevvdk | S100 S-100 calcium-binding |
| SEQ ID NO: 379 | protein beta subunit (S-100 |
| | protein, beta chain). |
| | (P04271 92 aa linear PRI 24- |
| | JAN.-2006) |
| eeikeqevvdkvmetldndg | S100 S-100 calcium-binding |
| SEQ ID NO: 380 | protein beta subunit (S-100 |
| | protein, beta chain). |
| | (P04271 92 aa linear PRI 24- |
| | JAN.-2006) |
| dkvmetldndgdgecdfqef | S100 S-100 calcium-binding |
| SEQ ID NO: 381 | protein beta subunit (S-100 |
| | protein, beta chain). |
| | (P04271 92 aa linear PRI 24- |
| | JAN.-2006) |
| dgdgecdfqefmafvamvtt | S100 S-100 calcium-binding |
| SEQ ID NO: 382 | protein beta subunit (S-100 |
| | protein, beta chain). |
| | (P04271 92 aa linear PRI 24- |
| | JAN.-2006) |
| efmafvamvttacheffehe | S100 S-100 calcium-binding |
| SEQ ID NO: 383 | protein beta subunit (S-100 |
| | protein, beta chain). |
| | (P04271 92 aa linear PRI 24- |
| | JAN.-2006) |
| tastmdharhgslpqkshgr | myelin basic protein hypo- |
| SEQ ID NO: 384 | thetical protein [Homo |
| | sapiens]. |
| | (CAH10359 74 aa linear PRI |
| | 19-AUG.-2004) |
| saytavsvlmpecmvfshgm | proteolipid protein PLP |
| SEQ ID NO: 385 | [Homo sapiens] |
| | (AAB26928 41 aa linear PRI |
| | 25-AUG.-199) |
| mpecmvfshgmlslarfvap | proteolipid protein PLP |
| SEQ ID NO: 386 | [Homo sapiens] |
| | (AAB26928 41 aa linear PRI |
| | 25-AUG.-199) |
| gmlslarfvaptfcpsakql | proteolipid protein PLP |
| SEQ ID NO: 387 | [Homo sapiens] |
| | (AAB26928 41 aa linear PRI |
| | 25-AUG.-199) |
| lpwnafpgkvccsnllsick | proteolipid protein proteo- |
| SEQ ID NO: 388 | lipid protein [Homo sapiens] |
| | (BAA02577 24 aa linear PRI |
| | 29-MAY-200) |
| qdwlqwhdhgslqpppprlk | Myelin-oligodendrocyte glyco- |
| SEQ ID NO: 389 | protein isoform beta4 |
| | precursor |
| | (NP_996536 171 aa) |
| aeggrekwnektsspgchfy | myelin basic protein Hypo- |
| SEQ ID NO: 390 | thetical protein FLJ44910 |
| | (Q6ZT70 154 aa linear PRI 05- |
| | JUL.-2004) |
| tkdsytlteelaeyaeirvk | myelin associated glycopro- |
| SEQ ID NO: 391 | tein Myelin associated glyco- |
| | protein isoform a variant. |
| | (Q53HD1 626 aa linear PRI 13- |
| | SEP.-2005) |
| iprgkeeleslgggktppgr | Myelin-oligodendrocyte glyco- |
| SEQ ID NO: 392 | protein MOG protein (Myelin |
| | oligodendrocyte glycoprotein). |
| | (Q8IYG5 295 aa linear PRI 13- |
| | SEP.-200) |
| fylrppsflrapsxfdtgls | crystallin Alpha B crystallin |
| SEQ ID NO: 393 | fragment 2. |
| | (Q9UC40 32 aa linear PRI 01- |
| | JUN-200) |
| awkvnasfllllnvvvmlav | Myelin-oligodendrocyte glyco- |
| SEQ ID NO: 394 | protein |
| | (NP_002535 440 aa linear PRI |
| | 24-SEP.-2005) |
| slpsclcsflllllqvsssy | Myelin-oligodendrocyte glyco- |
| SEQ ID NO: 395 | protein Myelin oligodendro- |
| | cyte glycoprotein isoform |
| | alpha1 variant. |
| | (Q59EJ6 259 aa linear PRI 10- |
| | MAY-2005) |
| ranpfrmgnhagkrelnaek | myelin basic protein Myelin |
| SEQ ID NO: 396 | basic protein variant |
| | (Q59GX3 203 aa linear PRI 10- |
| | MAY-200) |
| grffgsdraapkrgsgkdsh | myelin basic protein |
| SEQ ID NO: 397 | (P25188 167 aa linear ROD 24- |
| | JAN.2006 ACCESSION P25188 |
| | VERSION P25188 GI: 12679) |
| kpvptqgsrkggalqsctii | CNPase (ACCESSION P09543) |
| SEQ ID NO: 398 | |
| miaatynfavlklmgrgtkf | proteolipid protein Proteo- |
| SEQ ID NO: 399 | lipid protein 1, isoform 1. |
| | (Q502Y1 277 aa linear PRI 13- |
| | SEP.-2005) |
| kskgfkgahdaqgtlskifk | myelin basic protein |
| SEQ ID NO: 400 | (P25188 167 aa linear ROD 24- |
| | JAN.-2006 ACCESSION P25188 |
| | VERSION P25188 GI: 12679) |
| skifklggrdsrsgstmarr | myelin basic protein MBP |
| SEQ ID NO: 401 | protein. |
| | (Q6F104 160 aa linear PRI 05- |
| | JUL.-2004) |
| rmvsamnwrpgisslkitvw | Myelin-oligodendrocyte glyco- |
| SEQ ID NO: 402 | protein Myelin oligodendro- |
| | cyte glycoprotein. |
| | (Q5STM2 321 aa linear PRI 13- |
| | SEP.-2005) |
| gslspgtvemaslsrpslps | Myelin-oligodendrocyte glyco- |
| SEQ ID NO: 403 | protein Myelin oligodendro- |
| | cyte glycoprotein isoform |
| | alpha1 variant. |
| | (Q59EJ6 259 aa linear PRI 10- |
| | MAY-2005) |
| pqvkepkktgqfleellfhl | Myelin-oligodendrocyte glyco- |
| SEQ ID NO: 404 | protein Myelin oligodendro- |
| | cyte glycoprotein. |
| | (Q5SUK5 229 aa linear PRI 01- |
| | FEB.-2005) |
| pqvkepkktgqfleelrnpf | Myelin-oligodcndrocyte glyco- |
| SEQ ID NO: 405 | protein Myelin oligodendro- |
| | cyte glycoprotein. |
| | (Q5SUL0 224 aa linear PRI 01- |
| | FEB.-2005) |
| eienlhrtfgqfleelfhl | Myelin-oligodendrocyte glyco- |
| SEQ ID NO: 406 | protein Myelin oligodendro- |
| | cyte glycoprotein. |
| | (Q5SUK6 213 aa linear PRI 01- |
| | FEB.-2005) |
| eienlhrtfgqfleelmpf | Myelin-oligodendrocyte glyco- |
| SEQ ID NO: 407 | protein Myclin oligodendro- |
| | cyte glycoprotein. |
| | (Q5SUK9 208 aa linear PRI 01- |
| | FEB.-2005) |
| ffggdrgapkrgsgkvssee | myelin basic protein Myelin |
| SEQ ID NO: 408 | basic protein variant |
| | (Q59GX3 203 aa linear PRI 10- |
| | MAY-200) |
| ntpnivvplevvagtevevs | myelin associated glycopro- |
| SEQ ID NO: 409 | tein Myelin associated glyco- |
| | protein isoform a variant. |
| | (Q53HD1 626 aa linear PRI 13- |
| | SEP.-2005) |
| kyppvivevnssveaiegsh | myelin associated glycopro- |
| SEQ ID NO: 410 | tein Myelin associated glyco- |
| | protein isoform a variant. |
| | (Q53HD1 626 aa linear PRI 13- |
| | SEP.-2005) |
| pfqgahrltwakigpvgavv | myelin associated glycopro- |
| SEQ ID NO: 411 | tein Myelin associated glyco- |
| | protein isoform a variant. |
| | (Q53HD1 626 aa linear PRI 13- |
| | SEP.-2005) |
| vglsvmyaswkptvngtmva | myelin associated glycopro- |
| SEQ ID NO: 412 | tein Myelin associated glyco- |
| | protein isoform a variant. |
| | (Q53HD1 626 aa linear PRI 13- |
| | SEP.-2005) |
| yshsdlgkqptkdsytltee | myelin associated glycopro- |
| SEQ ID NO: 413 | tein Myelin associated glyco- |
| | protein isoform a variant. |
| | (Q53ES7 626 aa linear PRI 13- |
| | SEP.-2005) |
| yfntwttcdsiafpsktsas | proteolipid protein myelin |
| SEQ ID NO: 414 | proteolipid protein |
| | (AAA59565 277 aa linear PRI |
| | 07-JAN.-199) |
| rmygvlpwiafpgkvcgsnl | proteolipid protein myelin |
| SEQ ID NO: 415 | proteolipid protein |
| | (AAA59565 277 aa linear PRI |
| | 07-JAN.-199) |
| ahslervctclgkwlghpdk | proteolipid protein proteo- |
| SEQ ID NO: 416 | lipid protein |
| | (AAA60117 277 aa linear PRI |
| | 07-JAN.-1995) |
| ynwlhrrlagqfleelrnpf | Myelin-oligodendrocyte glyco- |
| SEQ ID NO: 417 | protein Myelin oligodendro- |
| | cyte glycoprotein isoform |
| | alpha1 variant. |
| | (Q59EJ6 259 aa linear PRI 10- |
| | MAY-2005) |
| llqitvgliflclqyrlrgk | Myelin-oligodendrocyte glyco- |
| SEQ ID NO: 418 | protein Myelin oligodendro- |
| | cyte glycoprotein. |
| | (Q5SUK7 252 aa linear PRI 01- |
| | FEB.-2005) |
| pvlllqitlglvflclqyrl | Myelin-oligodendrocyte glyco- |
| SEQ ID NO: 419 | protein |
| | (CAA88109 247 aa linear PRI |
| | 21-AUG.-1996) |
| aslvatglgffgvalfcgcg | proteolipid protein Proteo- |
| SEQ ID NO: 420 | lipid protein 1 (Pelizaeus- |
| | Merzbacher disease, spastic |
| | paraplegia 2, uncomplicated). |
| | (Q5U0F3 242 aa linear PRI |
| kkmpflelhflkqesfptse | crystallin unknown [Homo |
| SEQ ID NO: 421 | sapiens]. |
| | (AAC19161 194 aa linear PRI |
| | 08-JUN.-1998) |
| slarfvaptfcpsakqlssk | proteolipid protein PLP |
| SEQ ID NO: 422 | [Homo sapiens] |
| | (AAB26928 41 aa linear PRI |
| | 25-AUG.-199) |
| nafpgkvccsnllsicktae | proteolipid protein proteo- |
| SEQ ID NO: 423 | lipid protein [Homo sapiens] |
| | (BAA02577 24 aa linear PRI |
| | 29-MAY-200) |
| rptkdsytlteelaeccqgp | myelin associated glycopro- |
| SEQ ID NO: 424 | tein Myelin associated glyco- |
| | protein isoform a variant. |
| | (Q59GD9 661 aa linear PRI 10- |
| | MAY-2005) |
| eienlhrtfgqfleelpsri | Myelin-oligodendrocyte glyco- |
| SEQ ID NO: 425 | protein Myelin oligodendro- |
| | cyte glycoprotein. |
| | (Q5STM0 282 aa linear PRI 13- |
| | SEP.-2005) |
| icgkglsatfvgityaltvv | proteolipid protein PLP1 |
| SEQ ID NO: 426 | protein. |
| | (Q6FHZ6 242 aa linear PRI 10- |
| | MAY-200) |
| miaatynfailklmgrgtkf | proteolipid protein Proteo- |
| SEQ ID NO: 427 | lipid protein 1 (Pelizaeus- |
| | Merzbacher disease, spastic |
| | paraplegia 2, uncomplicated). |
| | (Q5U0F2 242 aa linear PRI |
| llqvsssyadpfywvspgvl | Myelin-oligodendrocyte glyco- |
| SEQ ID NO: 428 | protein Myelin oligodendro- |
| | cyte glycoprotein. |
| | (Q5STM1 205 aa linear PRI 13- |
| | SEP.-2005) |
| ggrdsrsgspmarrhhhhhh | myelin basic protein Myelin |
| SEQ ID NO: 429 | basic protein. |
| | (Q65ZS4 203 aa linear PRI 25- |
| | OCT.-2004) |
| ertiptireekpavtaapkk | crystallin Alpha crystallin B |
| SEQ ID NO: 430 | chain (Alpha(B)-crystallin) |
| | (Rosenthal fiber component) |
| | (Heat-shock protein beta-5) |
| | (HspB5). |
| | (P02511 175 aa |
| reggrrpetriwlrrqsvrl | myelin basic protein iso 5 |
| SEQ ID NO: 431 | (NP_001020265 137 aa linear |
| | PRI 18-OCT.-200) |
| rtpppsqgkreggrrpetri | myelin basic protein iso 6 |
| SEQ ID NO: 432 | (NP_001020269 126 aa linear |
| | PRI 18-OCT.-200) |
| rtpppsqgkgaegqrpgfgy | myelin basic protein myelin |
| SEQ ID NO: 433 | basic protein. |
| | (AAA59563 186 aa linear PRI |
| | 07-JAN.-199) |
| slpqksqrsqdenpvvhffk | myelin basic protein |
| SEQ ID NO: 434 | (P25188 167 aa linear ROD 24- |
| | JAN.-2006 ACCESSION P25188 |
| | VERSION P25188 GI: 12679) |
| mqfrvigprhpiralvgdev | Myelin-oligodendrocyte glyco- |
| SEQ ID NO: 435 | protein Myelin oligodendro- |
| | cyte glycoprotein. |
| | (Q4V357 154 aa linear PRI 13- |
| | SEP.-2005) |
| astmdharhgslpqkshgrt | myelin basic protein hypo- |
| SEQ ID NO: 436 | thetical protein [Homo |
| | sapiens]. |
| | (CAH10359 74 aa linear PRI |
| | 19-AUG.-2004) |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of individually optimizing a treatment for an inflammation associated disease, the method comprising:
(a) contacting each of identical white blood cell samples of a subject in need thereof with a different pharmaceutical agent of a plurality of pharmaceutical agents for the inflammation associated disease, so as to allow elicitation of an anti-inflammatory activity in said white blood cell samples;
(b) assaying said anti-inflammatory activity in said white blood cell samples; and
(c) identifying a pharmaceutical agent of said plurality of pharmaceutical agents eliciting a strongest anti-inflammatory activity, said pharmaceutical agent being the individually optimized treatment for the inflammation associated disease, wherein when said inflammation associated disease is multiple sclerosis said white blood cell samples are inflamed white blood cell samples.

2. A method of treating an inflammation associated disease in a subject, the method comprising:
(a) contacting each of identical white blood cell samples of the subject with a different pharmaceutical agent of a plurality of pharmaceutical agents for the inflammation associated disease, so as to allow elicitation of an anti-inflammatory activity in said white blood cell samples;
(b) assaying said anti-inflammatory activity in said white blood cell samples;
(c) identifying a pharmaceutical agent of said plurality of pharmaceutical agents eliciting a strongest anti-inflammatory activity, said pharmaceutical agent being the individually optimized treatment for the inflammation associated disease; and
(d) administering said pharmaceutical agent eliciting said strongest anti-inflammatory activity to said subject, wherein when said inflammation associated disease is multiple sclerosis, said white blood cell samples are inflamed white blood cell samples, thereby treating an inflammation associated disease in the subject.

3. A method of assessing the efficacy of a pharmaceutical agent for individually treating an inflammation associated disease, the method comprising:
(a) contacting a white blood cell sample of a subject in need thereof with a pharmaceutical agent for the inflammation associated disease, so as to allow elicitation of an anti-inflammatory activity in said white blood cell sample; and
(b) assaying said anti-inflammatory activity in said white blood cell samples, wherein an anti-inflammatory activity above a predetermined threshold is indicative of therapeutic efficacy of the pharmaceutical agent, wherein when said inflammation associated disease is multiple sclerosis, said white blood cell samples are inflamed white blood cell samples, thereby assessing the efficacy of a pharmaceutical agent for individually treating an inflammation associated disease.

4. The method of claim 1, wherein the inflammation associated disease is an autoimmune disease.

5. The method of claim 4, wherein said white blood cell samples are inflamed white blood cell samples.

6. The method of claim 5, further comprising contacting said white blood cell samples with at least one autoantigen of said autoimmune disease so as to obtain said inflamed blood cell samples prior to step (a).

7. The method of claim 6, wherein said at least one autoantigen is selected by:
(a) contacting a plurality of white blood cell samples of the subject with a plurality of peptides; and
(b) selecting at least one peptide of said plurality of peptides that elicits an immune activity above a predetermined threshold, said peptide being the autoantigen that activates white blood cells of the individual subject with said autoimmune disease.

8. (canceled)

9. The method of claim 4, wherein the subject is in remission from said autoimmune disease.

10.-12. (canceled)

13. The method of claim 4, wherein said auto-immune disease is multiple sclerosis.

14. The method of claim 4, wherein said autoimmune disease is Crohns disease.

15. The method of claim 13, wherein said pharmaceutical agent is selected from the group consisting of interferon-β-1-α, interferon-β-1-β, an immunoglobulin and glatiramer acetate.

16. The method of claim 14, wherein said pharmaceutical agent is selected from the group consisting of a 5A5A compound, sulfasalazine, mesalamine and olsalazine.

17. The method of claim 1, wherein said assaying anti-inflammatory activity comprises:
(i) assaying an activity and/or expression of an anti inflammatory cytokine;
(ii) assaying an activity and/or expression of a pro-inflammatory cytokine; and/or
(iii) assaying a ratio of (i) to (ii).

18. (canceled)

19. The method of claim 17, wherein said pro-inflammatory cytokine is TNF-α.

20.-24. (canceled)

25. The method of claim 13, wherein said at least one auto-antigen is selected from the proteins consisting of Myelin-associated Glycoprotein (MAG), Myelin-oligodendrocyte Glycoprotein (MOG), Myelin Basic Protein (MBP) and Proteolipid Protein (PLP).

26. The method of claim 13, wherein said at least one auto-antigen does not comprise more than 20 amino acids

27. The method of claim 26, wherein said amino acid peptides are selected from the group as set forth in Table 2.

28.-37. (canceled)

38. An array comprising a set of epitopes selected from the group of 20 amino acid peptides as set forth in Table 2.

39.-42. (canceled)

43. The method of claim 2, wherein the inflammation associated disease is an autoimmune disease.

44. The method of claim 43, wherein said white blood cell samples are inflamed white blood cell samples.

45. The method of claim 44, further comprising contacting said white blood cell samples with at least one autoantigen of said autoimmune disease so as to obtain said inflamed blood cell samples prior to step (a).

46. The method of claim 3, wherein the inflammation associated disease is an autoimmune disease.

47. The method of claim 46, wherein said white blood cell samples are inflamed white blood cell samples.

48. The method of claim 47, further comprising contacting said white blood cell samples with at least one autoantigen of said autoimmune disease so as to obtain said inflamed blood cell samples prior to step (a).
